(19) United States
(12) Patent Application Publication
(10) Pub. No.: US 2019/0099452 A1
(43) Pub. Date: Apr. 4, 2019

(54) METHODS FOR PROMOTING OLIGODENDROCYTE REGENERATION AND REMYELINATION

(71) Applicant: THE REGENTS OF THE UNIVERSITY OF CALIFORNIA, Oakland, CA (US)

(72) Inventors: Peng Jiang, Davis, CA (US); Wenbin Deng, Davis, CA (US)

(73) Assignees: THE REGENTS OF THE UNIVERSITY OF CALIFORNIA, Oakland, CA (US); SHRINERS HOSPITALS FOR CHILDREN, Tampa, FL (US)

(21) Appl. No.: 16/144,822

(22) Filed: Sep. 27, 2018

Related U.S. Application Data
(63) Continuation of application No. PCT/US2017/024834, filed on Mar. 29, 2017.
(60) Provisional application No. 62/316,413, filed on Mar. 31, 2016.
(60) Provisional application No. 62/314,863, filed on Mar. 29, 2016.

(51) Int. Cl. A61K 35/30; C12N 5/079; A61P 25/28; C12N 5/074; C12N 5/0735; C12Q 1/6851; C12Q 1/686

(52) U.S. Cl. CPC A61K 35/30; C12N 5/0622; A61P 25/28; C12N 5/0696; C12N 5/0606; C12Q 2600/158; C12Q 1/686; C12Q 2600/166; C12Q 2600/112; C12Q 2600/118; C12Q 1/6851

(57) ABSTRACT

The present invention provides a method for preventing or treating a demyelinating disease in a subject. Also provided herein is a method for reducing demyelination, inducing remyelination, promoting oligodendroglial progenitor cell (OPC) proliferation, and/or promoting oligodendrocyte differentiation in a subject. Kits are also described herein.

### CROSS-REFERENCE TO RELATED APPLICATIONS

The present application is a continuation of International Patent Application No. PCT/US2017/024834, filed Mar. 29, 2017, which claims priority to U.S. Provisional Application No. 62/314,863, filed Mar. 29, 2016, and U.S. Provisional Application No. 62/316,413, filed Mar. 31, 2016, the disclosures of which are herein incorporated by reference in their entirety for all purposes.

### STATEMENT AS TO RIGHTS TO INVENTIONS MADE UNDER FEDERALLY SPONSORED RESEARCH OR DEVELOPMENT

This invention was made with Government support under Grant No. R01HD087566, awarded by the National Institutes of Health. The government has certain rights in this invention.

### BACKGROUND OF THE INVENTION

Demyelinating diseases encompass a number of disorders that result in the degradation of the myelin sheath, producing a slowing or cessation of nerve cell conduction. The resulting neurological disorders are characterized by deficits in sensation, motor function, cognition, and other physiological functions. Signs and symptoms resulting from demyelinating diseases can range from the relatively mild to the profound, producing severe reductions in quality of life and possibly death. Multiple sclerosis, the most common demyelinating disease, affects several million people globally and is estimated to result in about 18,000 deaths per year. At present, there is no cure for demyelinating diseases. Accordingly, there is a need for new therapeutic approaches to the treatment of demyelinating diseases, including the promotion of remyelination and oligodendrocyte regeneration. The present invention satisfies this need, and provides related advantages as well.

### BRIEF SUMMARY OF THE INVENTION

In one aspect, the present invention provides a method for preventing or treating a demyelinating disease in a subject, the method comprising administering to the subject a therapeutically effective amount of immature astrocytes. In some embodiments, administration comprises transplanting the immature astrocytes into injured tissue in the subject. In other embodiments, about 1,000,000 to about 10,000,000 immature astrocytes are administered to the subject. In some instances, about 2,250,000 to about 4,500,000 immature astrocytes are administered to the subject.

In some embodiments, the immature astrocytes are suspended in a pharmaceutically acceptable carrier prior to administration. In some instances, the pharmaceutically acceptable carrier comprises phosphate-buffered saline. In some embodiments, the immature astrocytes are present at a concentration of about 50,000 to about 100,000 cells per microliter in the suspension.

In other embodiments, the demyelinating disease is selected from the group consisting of periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof. In some embodiments, the subject is a human.

In some embodiments, treating the subject reduces or eliminates one or more signs or symptoms of a demyelinating disease. In other embodiments, the subject does not have signs or symptoms of a demyelinating disease. In some embodiments, the subject has one or more risk factors for a demyelinating disease.

In particular embodiments, the immature astrocytes are not co-administered with another cell type. In some embodiments, the immature astrocytes are derived from a pluripotent stem cell. In some instances, the pluripotent stem cell is a human pluripotent stem cell. In some embodiments, the pluripotent stem cell is an induced pluripotent stem cell. In some instances, the induced pluripotent stem cell is derived from a cell obtained from the subject. In other embodiments, the pluripotent stem cell is an embryonic stem cell.

In some embodiments, the method further comprises determining the presence or level of one or biomarkers expressed by the immature astrocytes, wherein the presence or level of the one or more biomarkers is determined before administration. In some instances, the one or more biomarkers is selected from the group consisting of tissue inhibitor of metalloproteinase-1 (TIMP-1), glial fibrillary acidic protein (GFAP), S100 calcium-binding protein B (S100β), CD44, vimentin, nuclear factor 1 A-type (NF1A), excitatory amino acid transporter 1 (EAAT1), and a combination thereof. In particular embodiments, the presence or level of the one or more biomarkers is compared to a control. In some instances, the control is a mature astrocyte. In some embodiments, the level of TIMP-1, CD44, vimentin, NF1A, and/or GFAP is higher in the immature astrocytes than in the mature astrocyte control. In other embodiments, the level of EAAT1 is lower in the immature astrocytes than in the mature astrocyte control. In particular embodiments, the presence or level of the one or more biomarkers is determined by a method selected from the group consisting of immunohistochemistry, quantitative PCR, a glutamate transport assay, and a combination thereof.

In another aspect, the present invention provides a method for reducing demyelination, inducing remyelination, promoting oligodendroglial progenitor cell (OPC) proliferation, and/or promoting oligodendrocyte differentiation in a subject, the method comprising administering to the subject a therapeutically effective amount of immature astrocytes. In some embodiments, administration comprises transplanting the immature astrocytes into injured tissue in the subject. In other embodiments, about 1,000,000 to about 10,000,000 immature astrocytes are administered to the subject. In some instances, about 2,250,000 to about 4,500,000 immature astrocytes are administered to the subject.

In some embodiments, the immature astrocytes are suspended in a pharmaceutically acceptable carrier prior to administration. In some instances, the pharmaceutically acceptable carrier comprises phosphate-buffered saline. In other embodiments, the immature astrocytes are present at a concentration of about 50,000 to about 100,000 cells per microliter in the suspension. In some embodiments, the subject is a human.

In other embodiments, the subject has one or more risk factors for a demyelinating disease. In some embodiments, reducing demyelination, inducing remyelination, promoting oligodendroglial progenitor cell (OPC) proliferation, and/or promoting oligodendrocyte differentiation in the subject reduces or eliminates one or more signs or symptoms of a demyelinating disease. In other embodiments, the subject does not have signs or symptoms of a demyelinating disease. In some instances, the demyelinating disease is selected from the group consisting of periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof.

In particular embodiments, the immature astrocytes are not co-administered with another cell type. In some embodiments, the immature astrocytes are derived from a pluripotent stem cell. In particular embodiments, the pluripotent stem cell is a human pluripotent stem cell. In some embodiments, the pluripotent stem cell is an induced pluripotent stem cell. In some instances, the induced pluripotent stem cell is derived from a cell obtained from the subject. In other embodiments, the pluripotent stem cell is an embryonic stem cell.

In some embodiments, the method further comprises determining the presence or level of one or biomarkers expressed by the immature astrocytes, wherein the presence or level of the one or more biomarkers is determined before administration. In some instances, the one or more biomarkers is selected from the group consisting of tissue inhibitor of metalloproteinase-1 TIMP-1, glial fibrillary acidic protein (GFAP), S100 calcium-binding protein B (S100β), CD44, vimentin, nuclear factor 1 A-type (NF1A), excitatory amino acid transporter 1 (EAAT1), and a combination thereof. In some embodiments, the presence or level of the one or more biomarkers is compared to a control. In some instances, the control is a mature astrocyte. In particular embodiments, the level of TIMP-1, CD44, vimentin, NF1A, and/or GFAP is higher in the immature astrocytes than in the mature astrocyte control. In other embodiments, the level of EAAT1 is lower in the immature astrocytes than in the mature astrocyte control. In some embodiments, the presence or level of the one or more biomarkers is determined by a method selected from the group consisting of immunohistochemistry, quantitative PCR, a glutamate transport assay, and a combination thereof.

Other objects, features, and advantages of the present invention will be apparent to one of skill in the art from the following detailed description and figures.

### BRIEF DESCRIPTION OF THE DRAWINGS

FIGS. 1A-1F show immature astrocytes derived from hiPSCs and astrocytes in human brain tissue. FIG. 1A shows phase contrast images of hiPSC1 and hiPSC2 colonies cultured under feed and feeder-free conditions. Scale bars represent 250 μm. FIG. 1B shows a schematic procedure for differentiation of hiPSCs to immature astrocytes. FIG. 1C shows representatives of hiPSC1-Astros and hiPSC2-Astros expressing the astroglial markers s100β and GFAP. FIG. 1D shows representatives of hiPSC1-Astros and hiPSC2-Astros expressing hCD44 and vimentin, markers used to identify astrocyte-restricted precursor cells. FIG. 1E shows representative images showing that GFAP+ astrocytes expressed hCD44 in the frontal cerebral cortex of brain tissues from two normal individuals. Scale bars represent 50 μm. FIG. 1F shows representative images showing that S100β+ astrocytes expressed vimentin in the frontal cerebral cortex of brain tissues from two normal individuals. Scale bars represent 50 μm.

FIGS. 2A-2K show differentiation of hiPSC-Astros to human astrocytes with mature phenotypes. FIG. 2A shows representatives of GFAP⁺ and S100β⁺ hiPSC-Astros and hBrain-Astros. FIG. 2B shows OTX2-expressing cells in the S100β⁺ hiPSC-Astros and hBrain-Astros. FIG. 2C shows HOXB4-expressing cells in the S100β⁺ hiPSC-Astros and hBrain-Astros. FIG. 2D shows NKX2.1-expressing cells in the S100β⁺ hiPSC-Astros and hBrain-Astros. FIG. 2E shows representative images showing that expression of NF1A was abundant in immature hiPSC-Astros, but markedly reduced in mature hiPSC-Astros and hBrain-Astros. Insets show enlarged images from the corresponding squared areas. FIG. 2F shows representatives of hCD44- and vimentin-expressing cells in hiPSC-Astros, hBrain-Astros, and mature hiPSC-Astros. FIG. 2G shows the percentage of GFAP-, S100β-, OTX2-, HOXB4- and NKX2.1-expressing cells in hiPSC-Astros and hBrain-Astros (n=4 for each cell line). All quantitative data for hiPSC-Astros are analyses of pooled data collected from hiPSC1-Astros and hiPSC2-Astros. FIG. 2H shows qPCR analysis of NF1A, hCD44, and vimentin (Vim) mRNA expression in the three groups of human astrocytes (n=4 from each cell line). One-way ANOVA test, *P<0.05, **P<0.01 and ***P<0.001. FIG. 2I shows qPCR analysis of EAAT1 and EAAT2 mRNA expression in the three groups of human astrocytes (n=4 from each cell line). One-way ANOVA test, *P<0.05. FIG. 2J shows glutamate uptake analysis showing that mature hiPSC-Astros and hBrain-Astros exhibited glutamate uptake at a higher rate than immature hiPSC-Astros (n=4 from each cell line). This uptake capability was sodium-dependent and could be abolished with sodium-free solution. One-way ANOVA test, *P<0.05. FIG. 2K shows the proliferation rate of the three groups of human astrocytes relative to HEK293 cells (n=4 from each cell line). One-way ANOVA test, * P<0.05 and ***P<0.001. Scale bars, 50 μm. Nuclei were stained with DAPI.

FIGS. 3A-3F show the effects of immature and mature human astrocytes on OPC proliferation. FIG. 3A shows a schematic diagram showing that the primary mixed neuron/glia cultures at DIV 7 were treated with ACM and the proliferation of OPCs was examined 7 days after treatment of ACM (i.e., at DIV 14). FIG. 3B shows representatives of βIII-tubulin (βIIIT)⁺ neurons, GFAP⁺ astrocytes, and OPCs labeled by PDGFRα, NG2 and Olig2 in the mixed culture at DIV 7. There were no MBP⁺ oligodendrocytes in the culture. FIG. 3C shows representatives of Olig2⁺ oligodendroglial lineage cells and Ki67⁺ proliferating cells in the control (Cont) culture and cultures fed with hiPSC-Astro ACM, mature hiPSC-Astro ACM, and hBrain-Astro ACM. FIG. 3D shows representatives of Olig2⁺ and PDGFRα⁺ in the cultures fed with the different ACM. FIG. 3E shows the percentage of Olig2⁺ and Ki67⁺/Olig2⁺ cells in the cultures fed with the different ACM (n=3 from each cell line). All of the quantitative data for the hiPSC-Astros and mature hiPSC-Astros are analyses of pooled data collected from hiPSC1-Astros and hiPSC2-Astros, and mature hiPSC1-Astros and hiPSC2-Astros, respectively. One-way ANOVA test, *P<0.05, **P<0.01, and ***P<0.001, comparison between control group versus the groups treated with the different ACM; ^{##}P<0.01; and NS, not significant. FIG. 3F shows quantification of the percentage of PDGFRα⁺ cells in total Olig2⁺ cells (n=3 from each cell line). One-way ANOVA test, P>0.05. Scale bars, 50 μm.

FIGS. 4A-4E show the effects of immature and mature human astrocytes on OPC differentiation. FIG. 4A shows a schematic diagram showing that the differentiation of OPCs to oligodendrocytes in the primary mixed neuron/glia culture was examined after 14-day treatment of ACM (i.e., at DIV 21). FIG. 4B shows representatives of MBP⁺ oligodendrocytes and MAP2⁺ neurons in the Cont culture and the cultures fed with hiPSC-Astro ACM, mature hiPSC-Astro ACM, and hBrain-Astro ACM. The squared areas are enlarged. FIG. 4C shows representatives of Olig1⁺ oligodendroglial lineage cells and PDGFRα⁺ OPCs in the mix cultures fed with the different ACM. FIG. 4D shows quantification of pooled data showing the percentage of MBP⁺, Olig1⁺ and Olig1⁺/PDGFRα⁺ cells in the cultures fed with the different ACM (n=3 from each cell line). One-way ANOVA test, *P<0.05 and ***P<0.001, comparison between control group versus the groups treated with the different ACM; ^{###}P<0.001; and NS, not significant. FIG. 4E shows qPCR analysis of Olig1, Mbp, Plp, Cnp, and Pdgfra expression in the cultures fed with the different ACM (n=3 from each cell line). One-way ANOVA test, **P<0.01 and ***P<0.001, comparison between control group versus the groups treated with the different ACM; ^{##}P<0.01 and ^{###}P<0.001 and NS, not significant. Scale bars, 50 μm in the original and enlarged images. Nuclei were stained with DAPI.

FIGS. 5A and 5B show the effects of hESC-derived astrocytes on OPC differentiation. FIG. 5A shows representatives of MBP+ oligodendrocytes and βIII-tubulin+ (βIIIT) neurons in the primary mixed neuron/glia cultures fed with NPC-Astro ACM and Olig2PC-Astro ACM for 14 days (DIV21). Scale bars represent 50 μm. Nuclei were stained with DAPI. FIG. 5B shows quantification of the percentage of MBP+ oligodendrocytes in the cultures fed with the different ACM (n=3 for each group). One-way ANOVA test, ***P<0.001 and NS, not significant. Data are presented as mean ±S.E.M.

FIGS. 6A-6E show the effects of hiPSC-Astro ACM on purified primary OPCs. FIG. 6A shows representative images showing that the vast majority of the purified cells (i.e., about 85%) were OPCs, as indicated by expression of Olig2 and NG2. Scale bars represent 50 μm. FIG. 6B shows representatives of proliferating Olig2+/Ki67+ oligodendroglial cells in the control (Cont) culture and the culture fed with hiPSC-Astro ACM for 4 days. FIG. 6C shows quantification of the percentage of Olig2+ cells that were Olig2+/Ki67+ cells. Student's t test, *P<0.05. Data are presented as mean ±S.E.M. FIG. 6D shows representatives of MBP+ oligodendroglial lineage cells in the Cont culture and the culture fed with hiPSC-Astro ACM for 10 days. Scale bars represent 50 μm. FIG. 6E shows quantification of the percentage of MBP+ cells. Student's t test, **P<0.01. Data are presented as mean ±S.E.M.

FIGS. 7A-7I show the role of TIMP-1 in the effects of hiPSC-Astros on OPC differentiation. FIG. 7A shows gene expression analysis of immature hiPSC-Astros (hiPSC1-Astro and hiPSC2-Astro) and hESC-Astros (NPC-Astro and Olig2PC-Astro), focusing on gene transcripts encoding factors that are secreted by astrocytes and are involved in promoting OPC differentiation. FIG. 7B shows gene expression analysis of immature hiPSC-Astros (hiPSC1-Astro and hiPSC2-Astro) and hESC-Astros (NPC-Astro and Olig2PC-Astro), focusing on gene transcripts encoding factors that are secreted by astrocytes and are involved in inhibiting OPC differentiation. FIG. 7C shows gene expression analysis of immature hiPSC-Astros (hiPSC1-Astro and hiPSC2-Astro) and hESC-Astros (NPC-Astro and Olig2PC-Astro), focusing on gene transcripts encoding factors that are secreted by astrocytes and are involved in increasing OPC proliferation. FIG. 7D shows qPCR analysis of TIMP-1 expression in immature hiPSC-Astros and hESC-Astros, and in mature hiPSC-Astros, hESC-Astros, and hBrain-Astros (n=4 for each cell line). One-way ANOVA test, **P<0.01. FIG. 7E shows qPCR analysis of pooled data showing the expression of TIMP-1 in hiPSC-Astros at 48 hours after transfection with control (Cont) and TIMP-1 siRNA (n=3-5 from each line). Student's t test, **P<0.01. FIG. 7F shows representatives of MBP⁺ oligodendrocytes and MAP2⁺ neurons in the primary mixed neuron/glia cultures fed with Cont^{siRNA} ACM, TIMP-1^{siRNA} ACM, or TIMP-1^{siRNA} ACM supplemented with TIMP-1. The squared areas are enlarged. FIG. 7G shows representatives of Olig1⁺ and PDGFRα⁺ cells in the mixed cultures fed with the different ACM. FIG. 7H shows quantification of pooled data showing the percentage of MBP⁺, Olig1⁺ and Olig1⁺/PDGFRα⁺ cells in the cultures fed with the different ACM (n=3 from each line). One-way ANOVA test, *P<0.05. FIG. 7I shows qPCR analysis of pooled data showing Olig1, Mbp, Plp, Cnp, and Pdgfra expression in the cultures fed with the different ACM (n=3 from each line). One-way ANOVA test, *P<0.05 and **P<0.01. Scale bars, 50 μm. Nuclei were stained with DAPI.

FIGS. 8A-8G show myelination in the Rag1−/− mouse brains at P11 after PVL injury. FIG. 8A shows a representative image of GFAP+ cells in the brain at 4 days after PVL injury (P11). The squared areas labeled “B” and “C” are enlarged and shown in FIGS. 8B and 8C, respectively. Note that strong GFAP+ reactive astrocytes with hypertrophy morphology were seen in the ipsilateral side of the brain. Scar bars represents 200 μm. FIG. 8B shows an enlarged image of the region labeled “B” in FIG. 8A. Scale bar represents 50 μm. FIG. 8C shows an enlarged image of the region labeled “C” in FIG. 8A. Scale bar represents 50 μm. FIG. 8D shows representative images showing that at P11, the hN+ transplanted cells did not express Olig2. Scale bars represent 50 μm. FIG. 8E shows representatives of the node of Ranvier, identified by βIV spectrin+ staining flanked by Caspr+ staining, in the sham, vehicle, and hiPSC-Astro groups at P11. Animals in the sham group received sham PVL surgery and no vehicle PBS injection or cell transplantation. Notably, there were more nodes of Ranvier in the hiPSC-Astro and the sham groups than the vehicle group. Scale bar represents 10 μm. FIG. 8F shows representatives and quantification of the density of myelinated axons in the sham, vehicle and hiPSC-Astro groups (n=3 mice per group) at P11. Arrows indicate the myelinated axons. One-way ANOVA test, * P<0.05, comparison between vehicle group versus sham group; and # P<0.05 comparison between vehicle group versus hiPSC-Astro group. NS, not significant. Scale bars represent 2 μm. Data are presented as mean ±S.E.M. FIG. 8G shows representatives and quantification of fluorescence intensity (FI) of MBP staining in the vehicle and TIMP-1^{siRNA} hiPSC-Astro groups (n=4 for each group) at P11. Data represent the FI value normalized to the Contra side brain of the vehicle group. Student's t test, **P<0.01 and ***P<0.001, comparison between Ipsi side versus Contra side; NS, not significant. Data are presented as mean ±S.E.M.

FIGS. 9A-9H show transplantation of immature hiPSC-Astros into Rag1−/− mouse brains subjected to PVL. FIG. 9A shows a schematic diagram showing the timeline for the in vivo experiments from postnatal day (P) 6 to 60. FIG. 9B shows a cresyl violet-stained coronal brain section (left) at the level of the hippocampus, where analyses occurred. The red box outlines the white matter area used for immunohistochemical analyses of PVL insult. On the right is a diagram showing the cell transplantation site, which is adjacent to the injured white matter area. FIG. 9C shows representative images showing that grafted hiPSC-Astros were identified by human nuclei (hN) staining at P11. The transplanted hiPSC-Astros were also identified by hCD44, but negative for MBP staining. The squared areas in the middle panels are enlarged in the bottom panels. Arrowheads indicate the hN⁺/GFAP⁺ cells. CC, corpus callosum; LV, lateral ventricle; and HIP, hippocampus. FIG. 9D shows representative images (upper panels) showing that there were more Olig2⁺ oligodendroglial cells in the ipsilateral (Ipsi) side than in the contralateral (Contra) side brain in both vehicle and hiPSC-Astro groups. Transplantation of hiPSC-Astros further increased the expansion of Olig2⁺ cells in the Ipsi side. The lower panels show representative images showing that in vehicle group, MBP expression in the CC was only decreased substantially in the Ipsi side, with no significant changes in the Contra side. Transplantation of hiPSC-Astros promoted myelination in the Ipsi side and had no significant effect on the uninjured Contra side. FIG. 9E shows the number of Olig2⁺ cells in the analyzed area (n=4 for each group). Student's t test, **P<0.01, and ***P<0.001, comparison between the Ipsi side versus the Contra side; and ^{##}P<0.01. FIG. 9F shows fluorescence intensity (FI) of MBP staining (n=4 for each group). Data represent the FI value normalized to the Contra side brain of vehicle group. Student's t test, *P<0.05 and ***P<0.001, comparison between Ipsi side versus Contra side; and ^{#}P<0.05. FIG. 9G shows representatives and quantification of Olig2⁺/CC1⁺ and Olig2⁺/CC1⁻ cells in the Ipsi side brains of vehicle and hiPSC-Astro groups. Arrows indicate Olig2⁺/CC1⁺ cells and arrowheads indicate Olig2⁺/CC1⁻ cells. Student's t test, **P<0.01, and ***P<0.001, n=4 for each group. FIG. 9H shows representatives and quantification showing that the majority of hN⁺ transplanted hiPSC-Astros were positive for human TIMP-1 (hTIMP-1) staining at P11. Scar bars, 50 μm. n=4. Scale bars, 50 μm. Nuclei were stained with DAPI.

FIGS. 10A-10I show the effects of transplanted immature hiPSC-Astros on behavioral recovery and myelin ultrastructure. FIG. 10A shows the results of the Morris water-maze task that was performed at P60 to test the spatial learning ability of sham (n=7), vehicle (n=8), and hiPSC-Astro (n=10) transplanted groups, as shown by the time (escape latency) to find the submerged platform at navigation day 1 to 5. Two-way ANOVA test, *P<0.05, comparison between vehicle group versus sham group; and ^{#}P<0.05, comparison between vehicle group versus hiPSC-Astro group. FIG. 10B shows the results of probe trials that were performed 4 hours after the last maze trails on navigation day 5, monitored by relative radial-quadrant occupancy (time spent in the target radial-quadrant) (n=7-10). One way ANOVA test, *P<0.05, comparison between vehicle group versus sham group; ^{#}P<0.05, comparison between vehicle group versus hiPSC-Astro group; and NS, not significant. FIG. 10C shows representative sample paths from the maze trials ((1)-(3)) and the search patterns on the probe trials ((4)-(6)). (1),(4): sham group; (2),(5): vehicle group; (3),(6): hiPSC-Astro group. FIG. 10D shows low magnification electron micrographs showing a portion of corpus callosum from the animals in sham, vehicle and hiPSC-Astro groups. Scale bars, 2 μm. FIG. 10E shows an enlarged image from the vehicle group showing axons that have no compact myelin sheath (nma) among axons with myelin (ma). Scale bars, 500 nm. FIG. 10F shows the density of myelinated axons of each group (n=3 mice per group). One-way ANOVA test, *P<0.05, comparison between vehicle group versus sham group; and # P<0.05 comparison between vehicle group versus hiPSC-Astro group. NS, not significant. FIG. 10G shows representative electron micrographs in high magnification. Line “A” indicates the diameter of a myelinated axon fiber and line “a” indicates the diameter of axonal caliber. Scale bars, 0.25 μm. FIG. 10H shows a scatter plot graph showing the relationship between g ratio values and axon diameters in the three groups (n=3 mice per group). FIG. 10I shows the mean g ratio of the three groups (n=3 mice per group). One-way ANOVA test, **P<0.01, comparison between vehicle group versus sham group; and ^{##}P<0.01, comparison between vehicle group versus hiPSC-Astro group, and NS, not significant.

FIGS. 11A-11D show myelination of the Rag1−/− mouse brains at P60 after PVL injury. FIG. 11A shows representative images showing that at P60, the hiPSC-Astros were found close to the lateral ventricle (LV) and integrated into the hippocampus, close to the CA3 region. Notably, the majority of transplanted hiPSC-Astros expressed GFAP. s.o., stratum oriens; s.p., stratum pyramidale; and s.r., stratum radiatum. Scale bars represent 50 μm and 20 μm in original and enlarged images, respectively. FIG. 11B shows representatives images showing that the hN+ transplanted hiPSC-Astros were negative for human TIMP-1 (hTIMP-1) staining at P60. Scale bars represent 50 μm. FIG. 11C shows representatives of MBP staining in the Contra and Ipsi side brains from the vehicle and hiPSCAstro groups at P60 after PVL injury. CC, corpus callosum. Scale bars represent 50 μm. FIG. 11D shows quantification of fluorescence intensity (FI) of MBP staining (n=4 for each group). Data represent the FI value normalized to the Contra side brain of vehicle group. Student's t test, NS, not significant. Data are presented as mean ±S.E.M.

FIGS. 12A-12H show intranasal administration of ACM in a rat model of PVL. FIG. 12A shows a schematic diagram showing the timeline for intranasal administration of ACM from P8 to P11. FIG. 12B shows representatives of Olig2⁺ oligodendroglial cells and Olig2⁺/Ki67⁺ proliferating oligodendroglial cells, in Ipsi and Contra side brains from the rats that received intranasal administration of control (Cont) medium, Cont^{siRNA} ACM, TIMP-1^{siRNA} ACM, or mature hiPSC-Astro ACM. CC, corpus callosum. Arrowheads indicate the Olig2⁺/Ki67⁺ cells. FIG. 12C shows representatives of MBP expression in the CC of both Contra and Ipsi side brains from the four groups. FIG. 12D shows representatives of Olig2⁺ and NG2⁺ cells in the CC of the Ipsi side brains from Cont^{siRNA} ACM and TIMP-1^{siRNA} ACM groups. Arrowheads indicate the Olig2⁺/NG2⁺ cells. FIG. 12E shows the number of Olig2⁺ cells in the analyzed area (n=4 for each group). Student's t test, *P<0.05, and ***P<0.01, comparison between Ipsi side versus Contra side within the different groups; and ^{##}P<0.01 and ^{###}P<0.001, comparison between the Ipsi or Contra side from the Cont medium group versus that of the other groups. FIG. 12F shows the number of Olig2⁺/Ki67⁺ cells in the analyzed area (n=4 for each group). Student's t test, *P<0.05, and ***P<0.01, comparison between Ipsi side versus Contra side within the different groups; and ^{##}P<0.01 and ^{###}P<0.001, comparison between the Ipsi or Contra side from the Cont medium group versus that of the other groups. FIG. 12G shows fluorescence intensity (FI) of MBP staining (n=4 for each group). Data represent the FI value normalized to the Contra side brain of the Cont medium group. Student's t test, *P<0.05 and ***P<0.001, comparison between Ipsi side versus Contra side within the different groups; and ^{##}P<0.01, comparison between the Ipsi or Contra side from the Cont medium group versus that of the other groups. FIG. 12H shows the number of Olig2⁺/NG2⁺ cells in the CC of the Ipsi side brains from the Cont^{siRNA} ACM and TIMP-1^{siRNA} ACM groups (n=4 for each group). Student's t test, **P<0.01. Scar bars, 50 μm.

FIGS. 13A and 13B show ELISA analysis of human TIMP-1 protein levels. FIG. 13A shows quantification of the protein concentration of human TIMP-1 (hTIMP-1) in the different ACM and Cont medium (n=4 for each group). One-way ANOVA test, **P<0.01 and *** P<0.001. FIG. 13B shows quantification of protein concentration of hTIMP-1 in the P11 rat brains collected at 1 hour after the last intranasal administration of the different ACM or Cont medium. Human TIMP-1 concentrations in the olfactory bulb (OB) and frontal brain (FB) from the Cont^{siRNA} ACM group were significantly higher than those from the TIMP-1^{siRNA} ACM, mature hiPSC-Astro ACM, and Cont medium groups (n=4 for each group). There was no significance difference in the posterior brain (PB) among the groups. One-way ANOVA test, *P<0.05, **P<0.01, and *** P<0.001.

FIGS. 14A-14E show gene expression analysis of immature and mature mouse astrocytes. FIG. 14A shows a heat map of differential gene expression analysis of astrocytes derived from the brains of mouse at different postnatal days. The astrocytes clustered to two groups, immature (P1-P7) and mature astrocyte (P17-P30). A total of 1,161 differentially expressed genes (DEGs) were identified between the immature and mature astrocytes, including 650 upregulated genes and 511 downregulated genes in the immature astrocytes. FIG. 14B shows a heat map focusing on genes encoding astrocyte-secreted factors involved in promoting OPC differentiation. FIG. 14C shows a heat map focusing on genes encoding astrocyte-secreted factors involved in inhibiting OPC differentiation. FIG. 14D shows a heat map focusing on genes encoding astrocyte-secreted factors involved in increasing OPC proliferation. FIG. 14E shows a bar graph showing the DEGs encoding factors that promote OPC differentiation. Note that Tgfb2 had the highest expression level in immature mouse astrocytes.

### DETAILED DESCRIPTION OF THE INVENTION

### I. Introduction

Astrocytes play roles in myelination and are implicated in myelin loss disorders. While prior studies have demonstrated that oligodendrocytes preferentially remyelinate axons in areas containing astrocytes, astroglia-based therapy for myelin loss disorders has been less studied, in part because many of the relevant disorders are associated with profound astrocyte activation and the formation of glial scar. Scarring astrocytes have been regarded as a barrier to regeneration, partly due to their secretion of factors that halt the survival and differentiation of oligodendroglial progenitor cells (OPCs). Recent studies have suggested that in the acute phase of injuries, astrogliosis is a defensive reaction. While reactive astrocytes recapitulate numerous processes that are involved in the early development of immature astroglia and exhibit positive effects in the acute phase of injuries, reactivated processes often go awry later, turning on detrimental effects that astrocytes can have on regeneration.

Recent studies have demonstrated that transplanted immature astrocytes do not become reactive after CNS injury. Moreover, immature but not mature astrocytes are neuroprotective and suppress the activation of endogenous astrocytes and glial scar formation. Thus, in the context of preventing and treating demyelinating diseases, it is important to identify the proper population of cells for the promotion of oligodendrocyte regeneration and remyelination. The present invention is based, in part, on the discovery that immature astrocytes, but not mature astrocytes, can be transplanted on their own (i.e., without requiring co-transplantation of other cell types) and act on native oligodendrocyte progenitor cells (OPCs) to promote OPC proliferation, differentiation, and ultimately remyelination.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “an astrocyte” includes a plurality of such astrocytes and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “subject,” “individual,” and “patient” are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “administering” includes intravenous, intraperitoneal, intramuscular, intralesional, intracranial, intraparenchymal, intradermal, intralymphatic, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, and intraventricular. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment.

The term “therapeutically effective amount,” “effective amount,” or “sufficient amount” refers to the amount of immature astrocytes or other composition that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell or tissue type, the location of the target cell or tissue in the subject, the dosing regimen to be followed, whether it is administered in combination with other compounds, timing of administration, and the physical delivery system in which it is carried.

For the purposes herein a therapeutically effective amount is determined by such considerations as may be known in the art. The amount must be effective to achieve the desired therapeutic effect in a subject suffering from a demyelinating disease. The desired therapeutic effect may include, for example, amelioration of undesired symptoms associated with a demyelinating disease, prevention of the manifestation of such symptoms before they occur, slowing down the progression of symptoms associated with a demyelinating disease, slowing down or limiting any irreversible damage caused by a demyelinating disease, lessening the severity of or curing a demyelinating disease, or improving the survival rate or providing more rapid recovery from a demyelinating disease.

The therapeutically effective amount depends, inter alia, on the type and severity of the disease to be treated and the treatment regime. The therapeutically effective amount is typically determined in appropriately designed clinical trials (dose range studies) and the person versed in the art will know how to properly conduct such trials in order to determine the therapeutically effective amount. As generally known, a therapeutically effective amount depends on a variety of factors including the distribution profile of a therapeutic agent (e.g., an immature astrocyte or a plurality thereof) or composition within the body, the relationship between a variety of pharmacological parameters (e.g., half-life in the body) and undesired side effects, and other factors such as age and gender, etc.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, phosphate-buffered saline, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents, and buffers) or for preventing the action of microorganisms (e.g., antimicrobial and antifungal agents, such as parabens, chlorobutanol, sorbic acid, and the like). In some instances, the carrier is an agent that facilitates the delivery of an immature astrocyte to a target cell or tissue. One of skill in the art will recognize that other pharmaceutical carriers are useful in the present invention.

The term “demyelinating disease” refers to any condition that results in the reduction of or damage to the myelin sheath that surrounds nerve axons. Myelin is a fatty substance that forms an electrically insulating layer, which is essential for normal conduction of electrical signals along axons. Demyelination can lead to dramatically slowed or failed nerve signal conduction resulting in deficiencies in sensation, movement, cognition, and other functions. Demyelinating diseases are traditionally classified as either demyelinating myelinoclastic or demyelinating leukodystrophic diseases. Demyelinating myelinoclastic diseases include those wherein normal and healthy myelin is destroyed by a toxic, chemical, or autoimmune substance. Demyelinating leukodystrophic diseases include those in which the myelin is abnormal and undergoes degeneration. Non-limiting examples of demyelinating diseases include periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, transverse myelitis, acute disseminated encephalomyelitis, and cerebral palsy.

Demyelinating diseases have been associated with both genetic and environmental risk factors, and can be caused by chemical exposure, exposure to infectious agents, and autoimmune reactions. In some cases, the cause of a demyelinating disease is not known. Certain neuroleptic drugs, weed killers, and flea treatment preparations have been associated with the development of demyelinating diseases. Risk factors include age, sex, family history, certain previous infections, face, climate, certain autoimmune diseases, and tobacco smoking.

Signs and symptoms of demyelinating diseases include but are not limited to double vision, ataxia, clonus, dysarthria, fatigue, clumsiness, hand paralysis, hemiparesis, genital anesthesia, incoordination, paresthesia, ocular paralysis, impaired muscle coordination, muscle weakness, impaired sensation, impaired vision, unsteady gait, spastic paraparesis, incontinence, impaired hearing, speech problems, and impaired cognition.

Demyelinating diseases can be diagnosed by a variety of methods, including but not limited to magnetic resonance imaging (MRI), electrophysiological recording of evoked potentials (e.g., electroencephalography (EEG), electromyography (EMG)), cerebrospinal fluid (CSF) analysis (e.g., to detect microorganisms causing an infection leading to demyelination), and quantitative proton magnetic resonance spectroscopy (MRS).

The term “astrocyte,” also collectively referred to as “astroglia,” refers to a diverse population of glial cells found in the brain and spinal cord that are often star-shaped. Astrocytes perform functions such as repair and scarring processes, maintenance of extracellular ion balance (including removal of excess glutamate), provision of nutrients to nervous tissue, and provision of biochemical support to endothelial cells that make up the blood brain barrier. Astrocytes also supply glutamine in support of glutamatergic neurotransmission, control local neuronal blood flow, and promote oligodendrocyte proliferation and differentiation, thus promoting myelination. Astrocytes commonly express glial fibrillary acidic protein (GFAP).

Astrocytes can be classified in several different ways. Under one system, astrocytes are classified antigenically as either Type 1 of Type 2 astrocytes. Type 1 astrocytes are positive for rat neural antigen 2 (Ran2), GFAP, and fibroblast growth factor receptor 3 (FGFR3), and are negative for A2B5. These astrocytes can arise from the tripotential glial restricted precursor cells (GRPs) but not from the bipotential oligodendrocyte, type 2 astrocyte precursor (O2A/OPC) cells. Type 2 astrocytes are positive for GFAP and A2B5 and are negative for Ran2 and FGFR3. These astrocytes can arise from either GRPs or O2A cells.

Astrocytes can also be classified anatomically. Under this system, astrocytes are classified as either protoplasmic, Gomori-positive, or fibrous. Protoplasmic astrocytes are found in grey matter, and possess many branching processes, the feet of which develop synapses. Gomori-positive astrocytes are a subset of protoplasmic astrocytes and contain a large number of granules that stain positively with Gomori's chrome-alum hematoxylin stain. These astrocytes are more abundant in the arcuate nucleus of the hypothalamus and the hippocampus than other regions of the brain. Fibrous astrocytes are found in white matter and have long, thin unbranched processes, the feet of which develop nodes of Ranvier.

Alternatively, astrocytes can be classified according to transporter/receptor type. GluT type astrocytes express glutamate transporters (e.g., EAAT1 and EAAT2) and respond to the synaptic release of glutamate by transporter currents. GluR type astrocytes express glutamate receptors (i.e., mostly mGluR and AMPA type receptors) and respond to the synaptic release of glutamate by channel-mediated currents and IP3-depdendent calcium transients.

The term “immature astrocyte” refers to an astrocyte that is less differentiated than a mature astrocyte counterpart. The term includes astrocytes that exhibit morphological or functional properties that are present in astrocyte precursor cells (e.g., pluripotent stem cells such as induced pluripotent stem cells and embryonic stem cells) and/or are absent in mature astrocytes. The term also includes astrocytes that express biomarkers that are not expressed, or are expressed to a lesser degree, in mature astrocytes. In some cases, the biomarkers are also expressed in astrocyte precursor cells. In addition, the term includes astrocytes that do not express, or express to a lesser degree, biomarkers that are expressed in mature astrocytes. Expression can be measured, for example, at the mRNA transcript or protein level. As a non-limiting example, immature astrocytes can express TIMP-1, CD44, vimentin, NF1A, and/or GFAP at a higher level than mature astrocytes. As another non-limiting example, immature astrocytes can express EAAT1 at a lower level than mature astrocytes.

The terms “oligodendrocyte progenitor cell,” “oligodendroglial progenitor cell,” and “OPC,” also known as “NG2-glia” and “polydendrocyte,” refer to a subtype of glial cell that is a precursor to oligodendrocytes. OPCs can also differentiate into neurons and astrocytes. OPCs are commonly characterized by the expression of platelet derived growth factor receptor alpha (PDGFRA), chondroitin sulfate proteoglycan 4 (CSPG4; also known as NG2), and oligodendrocyte lineage transcription factor 2 (OLIG2). OPCs originate in brain and spinal neuroepithelium, then migrate to other regions of the CNS. Subsequently, OPCs differentiate into less mobile pro-oligodendrocytes, and then into oligodendrocytes. Differentiation into oligodendrocytes is accompanied by the expression of myelin basic protein (MBP), proteolipid protein (PLP), or myelin-associated glycoprotein (MAG).

The term “oligodendrocyte” refers to a type of glial cell that arises from the differentiation of oligodendrocyte progenitor cells. The primary function of oligodendrocytes is to provide support and electrical insulation for axons in the CNS. Electrical insulation is provided by forming a myelin sheath that wraps around the axon. A single oligodendrocyte can extend its processes to as many as 50 axons, and typically forms one segment of the myelin sheath for several adjacent axons. Oligodendrocytes also provide axonal trophic support by producing glial cell line-derived neurotrophic factor (GDNF), brain-derived neurotrophic factor (BDNF), and insulin-like growth factor-1 (IGF1).

The term “pluripotent stem cell” refers to a stem cell that possesses the ability to differentiate into a cell type that is derived from any of the three germ layers (i.e., endoderm, mesoderm, and ectoderm). Pluripotent stem cells include embryonic stem cells and induced pluripotent stem cells.

The term “induced pluripotent stem cell” or “iPSC” refers to a pluripotent stem that is derived from a non-pluripotent cell. Commonly, an adult somatic cell (e.g., a fibroblast, keratinocyte, liver cell, stomach cell, neural stem cell) is induced into the pluripotent state. Induction often comprises activating expression of certain genes and transcription factors. As a non-limiting example, fibroblasts can be reprogrammed to become iPSCs by retroviral transduction of the transcription factors Oct4, Sox2, Klf4, and c-myc. Cell reprogramming can be achieved by, for example, using single cassette vectors with Cre-Lox mediated transgene excision, nonintegrating viruses (e.g., adenovirus, Sendai virus), mRNA infection and/or transfection, PiggyBac mobile genetic elements, minicircle vectors, or episomal plasmids. More information regarding methods of iPSC derivation can be found, for example, in Malik et al. Methods Mol. Biol. 997:23-22 (2013), hereby incorporated by reference in its entirety for all purposes.

The term “embryonic stem cell” or “ESC” refers to a pluripotent stem cell that is derived from the inner mass of a blastocyst (i.e., an early-stage pre-implantation embryo). In humans, embryos typically reach the blastocyst stage about four or five days after fertilization, at which time the blastocyst contains about 50 to 150 cells. In addition to being pluripotent (i.e., being able to ultimately differentiate into any of more than 220 types of cells in the body, derived from the three germ layers), embryonic stem cells are typically characterized by the ability to propagate, potentially indefinitely.

The term “glial fibrillary acidic protein” or “GFAP” refers to a protein that is encoded by the GFAP gene in humans and is an intermediate filament protein that is expressed by numerous CNS cell types, including astrocytes. In the context of astrocytes, GFAP plays roles in cell communication (e.g., cell-cell interactions between astrocytes and neurons) and forming the structure of the blood brain barrier. Non-limiting examples of human GFAP mRNA sequences are set forth under GenBank reference numbers NM_002055→NP_002046, NM_001131019→NP_001124491, and NM_001242376→NP_001229305.

The term “S100 calcium-binding protein B” or “S100β” refers to a member of S-100 protein family, which are proteins that are localized in both the cytoplasm and the nucleus in a wide range of cells. S100β is also known as S100B, NEF, S100, and S100-B. S100β is glial-specific, being expressed primarily by astrocytes. However, not all astrocytes express S100β. Functionally, S100β plays roles in astrocytosis, proliferation, inhibition of microtubule assembly, inhibition of PKC-mediated phosphorylation, and stimulation of calcium fluxes. Genetic rearrangements and altered expression of the gene encoding S100β have been implicated in neurological diseases such as Alzheimer's disease, epilepsy, Down's syndrome, amyotrophic lateral sclerosis, and schwannoma. S100β is secreted by astrocytes and is released from damaged cells. Serum S100β levels are often increased during the acute phase of brain injury. A non-limiting example of a human S100β mRNA sequence is set forth under GenBank reference number NM_006272→NP_006263.

The term “CD44” refers to a cell surface glycoprotein that is encoded by the CD44 gene on chromosome 11 in humans. CD44 is also known as CDW44, CSPG8, ECMR-III, HCAM, HCELL, HUTCH-I, IN, LHR, MC56, MDU2, MDU3, MIC4, and Pgp1. CD44 plays roles in a wide range of cellular processes, including lymphocyte activation, recirculation and homing, hematopoiesis, and tumor metastasis. CD44 is expressed in astrocyte-restricted precursor cells and astrocytes, and is more highly expressed in immature astrocytes than mature astrocytes. Non-limiting examples of human CD44 mRNA sequences are set forth under GenBank reference numbers NM_000610→NP_000601, NM_001001389→NP_001001389, NM_001001390→NP_001001390, NM_001001391→NP_001001391, and NM_001001392→NP_001001392.

The term “nuclear factor 1 A-type” or “NF1A” refers to a protein that is encoded by the NFIA gene in humans. NF1A is also known as CTF, NF-I/A, NFI-A, and NFI-L. NF1A is a member of the family of nuclear factor I proteins that are dimeric DNA-binding proteins. Nuclear factor I proteins function as transcription factors and replication factors in adenovirus. In its role as a transcription factor, NF1A specifies glial cell identity and promotes astrocyte differentiation. Furthermore, NF1A, together with NFIB and SOX9, can be used to reprogram fibroblasts for conversion into functional astrocytes. NF1A functions as an astrocyte maturity marker, being more highly expressed in immature astrocytes than in mature astrocytes. Non-limiting examples of human NF1A mRNA sequences are set forth under GenBank reference numbers NM_001134673→NP_001128145, NM_001145511→NP_001138983, NM_001145512→NP_001138984, and NM_005595→NP_005586.

The term “excitatory amino acid transporter 1” or “EAAT1” refers to a homotrimeric transporter that mediates the transport of glutamic acid and aspartic acid, with the co-transport of three sodium ions and one proton. EAAT1 is encoded by the SLC1A3 gene in humans, and is also known as EA6, GLAST, GLAST1, and solute carrier family 1 member 3. Of the five EEAT subtypes, EEAT1 and EEAT2 are expressed in the membranes of glial cells (including astrocytes, microglia, and oligodendrocytes). EAAT1 regulates extracellular glutamate concentrations and plays a neuroprotective role in the CNS. In addition, EAAT1 is more highly expressed in mature astrocytes than immature astrocytes. Non-limiting examples of human EAAT1 mRNA sequences are set forth under GenBank reference numbers NM_001166695→NP_001160167, NM_001166696→NP_001160168, NM_001289939→NP_001276868, NM_001289940→NP_001276869, and NM_004172→NP_004163.

The term “tissue inhibitor of metalloproteinase-1” or “TIMP-1” refers to a glycoprotein that is encoded by the TIMP1 gene and is a member of the TIMP family of proteins. TIMP-1 is also known as CLGI, EPA, EPO, HCI, TIMP, and TIMP metallopeptidase inhibitor 1. TIMP-1 is an inhibitor of matrix metalloproteinases, which are involved in the degradation of the extracellular matrix. TIMP-1 can also promote proliferation in a wide range of cell types. In the central nervous system, TIMP-1 promotes both astrocyte proliferation and oligodendrocyte progenitor cell (OPC) differentiation. TIMP-1 is more highly expressed in immature astrocytes than mature astrocytes, and plays a role in the ability of immature astrocytes to promote oligodendrocyte maturation. A non-limiting example of a human TIMP-1 mRNA sequence is set forth under GenBank reference number NM_003254→NP_003245.

### III. Detailed Description of the Embodiments

### A. Methods for Preventing or Treating Demyelinating Diseases

In one aspect, the present invention provides a method for preventing or treating a demyelinating disease in a subject, the method comprising administering to the subject a therapeutically effective amount of immature astrocytes. In some embodiments, the immature astrocytes are not co-administered with any other cell type (e.g., only immature astrocytes are administered, and they affect native cells in the subject).

In some embodiments, administration comprises transplanting immature astrocytes into injured tissue in the subject. In some instances, the tissue has experienced severe injury (e.g., severe demyelination). In other instances, the tissue has experienced moderate or mild injury (e.g., moderate or mild demyelination). The injury can be in the acute phase, or can have occurred in the past. In some instances, the tissue experienced injury (e.g., demyelination) about 1, 2, 3, 4, 5, 6, 7, 8, 9, 10, 11, 12, 13, 14, 15, 16, 17, 18, 19, 20, 21, 22, 23, 24, or more hours prior to administration. In some instances, the tissue experienced injury (e.g., demyelination) about 1, 2, 3, 4, 5, 6, 7, or more days prior to administration. In other instances, the tissue experienced injury (e.g., demyelination) about 1, 2, 3, 4, 5, 6, 7, 8, 9, 10, 11, 12, or more weeks prior to administration. In yet other instances, the tissue experienced injury (e.g., demyelination) about 1, 2, 3, 4, 5, 6, 7, 8, 9, 10, 11, 12, or more months prior to administration. In some instances, the tissue experienced injury (e.g., demyelination) about 1, 2, 3, 4, 5, 6, 7, 8, 9, 10, or more years prior to administration.

In some embodiments, the immature astrocytes are transplanted directly into injured tissue. In other embodiments, the immature astrocytes are transplanted into tissue that is proximal to the site of injury. In some instances, the immature astrocytes are transplanted about 1 to 10 millimeters (e.g., about 1, 2, 3, 4, 5, 6, 7, 8, 9, or 10 millimeters) away from the site of injury. In other instances, the immature astrocytes are transplanted about 1 to 20 centimeters (e.g., about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 centimeters) away from the site of injury. In some instances, the immature astrocytes are transplanted to a site that is more than about 20 centimeters away from the site of injury. In particular embodiments, the immature astrocytes are transplanted into tissue that is uninjured. As a non-limiting example, it may be determined that a particular tissue is at risk for injury, and the immature astrocytes are transplanted into the tissue as a prophylactic measure.

In some embodiments, the immature astrocytes are transplanted into central nervous system (CNS) tissue. In some instances, the CNS tissue is brain tissue. In other instances, the CNS tissue is spinal cord tissue. In particular instances, the immature astrocytes are transplanted into both brain and spinal cord tissue.

Methods of the present invention are suitable for treating any demyelinating disease. In some embodiments, the demyelinating disease is selected from the group consisting of periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof.

In some embodiments, treating the subject reduces or eliminates one or more signs or symptoms of a demyelinating disease. The signs or symptoms can be related to, for example, deficits in sensory and/or motor function, cognition, or other physiological functions. In some embodiments, administration of the immature astrocytes to the subject prevents or improves, for example, weakness, paralysis, degraded vision, incoordination, paresthesias, impaired muscle coordination, difficulties with speech and/or hearing, incontinence, impaired cognition, or any other sign or symptom of a demyelinating disease described herein. Changes or improvements in function, signs, or symptoms can be measured by a variety of methods that will be known to one of skill in the art. Such methods include, as non-limiting examples, magnetic resonance imaging (MRI), magnetic resonance spectrography (MRS), electrophysiological recording (e.g., electroencephalography (EEG), electromyography (EMG)), cerebrospinal fluid (CSF) analysis, and various tests of cognition and muscle coordination.

In some embodiments, the subject does not have signs or symptoms of a demyelinating disease. In particular embodiments, the subject has one or more risk factors for a demyelinating disease. Risk factors can be, for example, genetic or environmental (e.g., the subject has been exposed to particular chemical and/or infectious agents that are associated with a demyelinating disease), or can be related to a prior history of an autoimmune disease.

In some embodiments, administration of the immature astrocytes prolongs the subject's survival time. The term “survival time” refers to a length of time following the diagnosis of a disease and/or beginning or completing a particular course of therapy for a disease (e.g., a demyelinating disease). The term “overall survival” includes the clinical endpoint describing patients who are alive for a defined period of time after being diagnosed with or treated for a disease, such as a demyelinating disease. The term “disease-free survival” includes the length of time after treatment for a specific disease (e.g., a demyelinating disease) during which a patient survives with no sign of the disease (e.g., without known recurrence). In certain embodiments, disease-free survival is a clinical parameter used to evaluate the efficacy of a particular therapy, which is usually measured in units of 1 or 5 years. The term “progression-free survival” includes the length of time during and after treatment for a specific disease (e.g., a demyelinating disease) in which a patient is living with the disease without additional symptoms of the disease. In some embodiments, survival is expressed as a median or mean value. In some instances, the survival time is increased by about 1, 2, 3, 4, 5, 6, 7, 8, 9, 10, 11, 12, or more weeks. In other instances, the survival time is increased by about 1, 2, 3, 4, 5, 6, 7, 8, 9, 10, 11, 12, or more months. In some instances, the survival time is increased by about 1, 2, 3, 4, 5, 6, 7, 8, 9, 10, or more years.

B. Methods for Reducing Demyelination, Inducing Remyelination, Promoting OPC Proliferation, and/or Oligodendrocyte Differentiation

In another aspect, the present invention provides a method for reducing demyelination, inducing remyelination, promoting oligodendroglial progenitor cell (OPC) proliferation, and/or promoting oligodendrocyte differentiation in a subject, the method comprising administering to the subject a therapeutically effective amount of immature astrocytes. In some embodiments, the immature astrocytes are not co-administered with any other cell type (e.g., only immature astrocytes are administered, and they affect native cells in the subject).

In some embodiments, administration comprises transplanting immature astrocytes into injured tissue in the subject. In some embodiments, the immature astrocytes exert effects on cells (e.g., OPCs) in the region of tissue injury, such that demyelination is attenuated or arrested. In other embodiments, the immature astrocytes exert effects on cells (e.g., OPCs) in the region of tissue injury, such that remyelination or myelination of new cells is induced. In some other embodiments, the immature astrocytes exert effects on cells in the region of tissue injury, such that OPC proliferation and/or oligodendrocyte differentiation is promoted. In some instances, the tissue has experienced severe injury (e.g., severe demyelination). In other instances, the tissue has experienced moderate or mild injury (e.g., moderate or mild demyelination). The injury can be in the acute phase, or can have occurred in the past. In some instances, the tissue experienced injury (e.g., demyelination) about 1, 2, 3, 4, 5, 6, 7, 8, 9, 10, 11, 12, 13, 14, 15, 16, 17, 18, 19, 20, 21, 22, 23, 24, or more hours prior to administration. In some instances, the tissue experienced injury (e.g., demyelination) about 1, 2, 3, 4, 5, 6, 7, or more days prior to administration. In other instances, the tissue experienced injury (e.g., demyelination) about 1, 2, 3, 4, 5, 6, 7, 8, 9, 10, 11, 12, or more weeks prior to administration. In yet other instances, the tissue experienced injury (e.g., demyelination) about 1, 2, 3, 4, 5, 6, 7, 8, 9, 10, 11, 12, or more months prior to administration. In some instances, the tissue experienced injury (e.g., demyelination) about 1, 2, 3, 4, 5, 6, 7, 8, 9, 10, or more years prior to administration.

In some embodiments, the immature astrocytes are transplanted directly into injured tissue. In other embodiments, the immature astrocytes are transplanted into tissue that is proximal to the site of injury. In some instances, the immature astrocytes are transplanted about 1 to 10 millimeters (e.g., about 1, 2, 3, 4, 5, 6, 7, 8, 9, or 10 millimeters) away from the site of injury. In other instances, the immature astrocytes are transplanted about 1 to 20 centimeters (e.g., about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 centimeters) away from the site of injury. In some instances, the immature astrocytes are transplanted to a site that is more than about 20 centimeters away from the site of injury. In particular embodiments, the immature astrocytes are transplanted into tissue that is uninjured. As a non-limiting example, it may be determined that a particular tissue is at risk for injury, and the immature astrocytes are transplanted into the tissue as a prophylactic measure (e.g., to induce remyelination, promote OPC proliferation, and/or promote oligodendrocyte differentiation).

In some embodiments, the immature astrocytes are transplanted into central nervous system (CNS) tissue. In some instances, the CNS tissue is brain tissue. In other instances, the CNS tissue is spinal cord tissue. In particular instances, the immature astrocytes are transplanted into both brain and spinal cord tissue.

In some embodiments, reducing demyelination, inducing remyelination, promoting OPC proliferation, and/or promoting oligodendrocyte differentiation in the subject reduces or eliminates one or more signs or symptoms of a demyelinating disease. While any demyelinating disease can be the target of reducing demyelination, inducing remyelination, promoting OPC proliferation, and/or promoting oligodendrocyte differentiation according to methods of the present invention, non-limiting examples include periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof.

The signs or symptoms can be related to, for example, deficits in sensory and/or motor function, cognition, or other physiological functions. In some embodiments, reducing demyelination, inducing remyelination, promoting OPC proliferation, and/or promoting oligodendrocyte differentiation in the subject prevents or improves, for example, weakness, paralysis, degraded vision, incoordination, paresthesias, impaired muscle coordination, difficulties with speech and/or hearing, incontinence, impaired cognition, or any other sign or symptom of a demyelinating disease described herein. Changes or improvements in function, signs, or symptoms can be measured by a variety of methods that will be known to one of skill in the art. Such methods include, as non-limiting examples, magnetic resonance imaging (MRI), magnetic resonance spectrography (MRS), electrophysiological recording (e.g., electroencephalography (EEG), electromyography (EMG)), cerebrospinal fluid (CSF) analysis, and various tests of cognition and muscle coordination.

In some embodiments, the subject does not have signs or symptoms of a demyelinating disease. In particular embodiments, the subject has one or more risk factors for a demyelinating disease. Risk factors can be, for example, genetic or environmental (e.g., the subject has been exposed to particular chemical and/or infectious agents that are associated with a demyelinating disease), or can be related to a prior history of an autoimmune disease.

In some embodiments, reducing demyelination, inducing remyelination, promoting OPC proliferation, and/or promoting oligodendrocyte differentiation in a subject prolongs the subject's survival time. The term “survival time” refers to a length of time following the diagnosis of a disease and/or beginning or completing a particular course of therapy for a disease (e.g., a demyelinating disease). The term “overall survival” includes the clinical endpoint describing patients who are alive for a defined period of time after being diagnosed with or treated for a disease, such as a demyelinating disease. The term “disease-free survival” includes the length of time after treatment for a specific disease (e.g., a demyelinating disease) during which a patient survives with no sign of the disease (e.g., without known recurrence). In certain embodiments, disease-free survival is a clinical parameter used to evaluate the efficacy of a particular therapy, which is usually measured in units of 1 or 5 years. The term “progression-free survival” includes the length of time during and after treatment for a specific disease (e.g., a demyelinating disease) in which a patient is living with the disease without additional symptoms of the disease. In some embodiments, survival is expressed as a median or mean value. In some instances, the survival time is increased by about 1, 2, 3, 4, 5, 6, 7, 8, 9, 10, 11, 12, or more weeks. In other instances, the survival time is increased by about 1, 2, 3, 4, 5, 6, 7, 8, 9, 10, 11, 12, or more months. In some instances, the survival time is increased by about 1, 2, 3, 4, 5, 6, 7, 8, 9, 10, or more years.

### C. Administration

According to the methods set forth herein for preventing or treating a demyelinating disease in a subject, or reducing demyelination, inducing remyelination, promoting oligodendroglial progenitor cell (OPC) proliferation, and/or promoting oligodendrocyte differentiation in a subject, the methods comprise administering to the subject a therapeutically effective amount of immature astrocytes.

In some embodiments, the therapeutically effective amount comprises between about 100 and about 10,000 (e.g., about 100, 150, 200, 250, 300, 350, 400, 450, 500, 550, 600, 650, 700, 750, 800, 900, 950, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000) immature astrocytes. In other embodiments, the therapeutically effective amount comprises between about 10,000 and about 100,000 (e.g., about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000) immature astrocytes. In some other embodiments, the therapeutically effective amount comprises between about 100,000 and about 1,000,000 (e.g., about 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000) immature astrocytes. In some embodiments, the therapeutically effective amount comprises at least about 1,000,000 to about 10,000,000 (e.g., about 1,000,000, 1,500,000, 2,000,000, 2,500,000, 3,000,000, 3,500,000, 4,000,000, 4,500,000, 5,000,000, 5,500,000, 6,000,000, 6,500,000, 7,000,000, 7,500,000, 8,000,000, 8,500,000, 9,000,000, 9,500,000, 10,000,000, or more) immature astrocytes. In particular embodiments, the therapeutically effective amount comprises between about 2,250,000 and about 4,500,000 (e.g., about 2,250,000, 2,500,000, 2,750,000, 3,000,000, 3,250,000, 3,500,00, 3,750,000, 4,000,000, 4,250,000, or 4,500,000) immature astrocytes.

According to methods of the present invention, immature astrocytes can be administered to a subject as part of a pharmaceutical composition. In some embodiments, the pharmaceutical composition comprises the immature astrocytes (either a therapeutically effective amount of immature astrocytes, or a smaller amount if the therapeutically effective amount is to be administered as multiple doses) and a pharmaceutically acceptable carrier. The formulation of pharmaceutical compositions is generally known in the art (see, e.g., Remington's Pharmaceutical Sciences, 18th Ed., Mack Publishing Co., Easton, Pa. (1990)). Prevention against microorganism contamination can be achieved through the addition of one or more of various antibacterial and antifungal agents.

In some embodiments, the pharmaceutical composition takes the form of a suspension of immature astrocytes. In particular embodiments, the immature astrocytes are suspended in phosphate-buffered saline. In some embodiments, the immature astrocytes are present in the suspension at a concentration of about 100 to about 1,000 cells per microliter (e.g., about 100, 150, 200, 250, 300, 350, 400, 450, 500, 550, 600, 650, 700, 750, 800, 850, 900, 950, or 1,000 cells per microliter). In other embodiments, the immature astrocytes are present at a concentration of about 1,000 to about 10,000 cells per microliter (e.g., about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000 cells per microliter). In still other embodiments, the immature astrocytes are present at a concentration of about 10,000 to about 100,000 cells per microliter (e.g., about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 cells per microliter). In some other embodiments, the immature astrocytes are present at a concentration of at least about 100,000 to about 500,000 cells per microliter (e.g., about 100,000, 150,000, 200,000, 250,000, 300,000, 350,000, 400,000, 450,000, 500,000, or more cells per microliter). In certain embodiments, the immature astrocytes are present at a concentration of about 50,000 to about 100,000 cells per microliter (e.g., about 50,000, 51,000, 52,000, 53,000, 54,000, 55,000, 56,000, 57,000, 58,000, 59,000, 60,000, 61,000, 62,000, 63,000, 64,000, 65,000, 66,000, 67,000, 68,000, 69,000, 70,000, 71,000, 72,000, 73,000, 74,000, 75,000, 76,000, 77,000, 78,000, 79,000, 80,000, 81,000, 82,000, 83,000, 84,000, 85,000, 86,000, 87,000, 88,000, 89,000, 90,000, 91,000, 92,000, 93,000, 94,000, 95,000, 96,000, 97,000, 98,000, 99,000, or 100,000 cells per microliter).

Pharmaceutical forms suitable for administration include but are not limited to sterile aqueous suspensions, solutions, and dispersions. In addition to those described elsewhere herein, suitable carriers include a solvent or dispersion medium containing, for example, water, NaCl, water-buffered aqueous solutions (i.e., biocompatible buffers, non-limiting examples of which include normal saline, phosphate-buffered saline, and Lactated Ringer's solution), ethanol, polyols (such as glycerol, propylene glycol, polyethylene glycol, and suitable mixtures thereof), surfactants, or vegetable oils.

Sterilization can be accomplished by any art-recognized technique, including but not limited to addition of antibacterial or antifungal agents, for example, paraben, chlorobutanol, sorbic acid or thimerosal. Further, isotonic agents such as sugars or sodium chloride may be incorporated in the subject compositions.

Production of sterile injectable solutions containing immature astrocytes, and/or other composition(s) can be accomplished by incorporating the immature astrocytes and/or other composition(s) in the required amount(s) in the appropriate solvent with various ingredients enumerated above, as required, followed by sterilization.

In some embodiments, the immature astrocytes, and/or other composition(s) provided herein are formulated for administration, e.g., intraparenchymal injection, intracranial injection, intraspinal injection, intrathecal injection, intradermal injection, intralymphatic injection, nasal, or parental administration in unit dosage form for ease of administration and uniformity of dosage. Unit dosage forms, as used herein, refers to physically discrete units suited as unitary dosages for the subjects, e.g., humans or other mammals to be treated, each unit containing a predetermined quantity of active material calculated to produce the desired therapeutic effect in association with the required pharmaceutical carrier. In some instances, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the immature astrocytes, and/or other composition(s).

A dose may include a therapeutically effective amount of immature astrocytes, or may contain less than a therapeutically effective amount of immature astrocytes (e.g., when it is necessary or desirable to administer the therapeutically effective amount over one or more doses. In some embodiments, a dose comprises between about 100 and about 10,000 (e.g., about 100, 150, 200, 250, 300, 350, 400, 450, 500, 550, 600, 650, 700, 750, 800, 900, 950, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or 10,000) immature astrocytes. In other embodiments, a dose comprises between about 10,000 and about 100,000 (e.g., about 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000) immature astrocytes. In some other embodiments, a dose comprises between about 100,000 and about 1,000,000 (e.g., about 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000) immature astrocytes. In some embodiments, a dose comprises at least about 1,000,000 to about 10,000,000 (e.g., about 1,000,000, 1,500,000, 2,000,000, 2,500,000, 3,000,000, 3,500,000, 4,000,000, 4,500,000, 5,000,000, 5,500,000, 6,000,000, 6,500,000, 7,000,000, 7,500,000, 8,000,000, 8,500,000, 9,000,000, 9,500,000, 10,000,000, or more) immature astrocytes. In particular embodiments, a dose comprises between about 2,250,000 and about 4,500,000 (e.g., about 2,250,000, 2,500,000, 2,750,000, 3,000,000, 3,250,000, 3,500,00, 3,750,000, 4,000,000, 4,250,000, or 4,500,000) immature astrocytes.

When immature astrocytes are administered as a suspension, solution, dispersion, or other composition, the volume of the suspension, solution, dispersion, or other composition to be administered will depend on the number of cells to be administered and the desired cell (i.e., immature astrocyte) concentration. In some embodiments, the volume of the suspension, solution, dispersion, or other composition to be administered is about 1 to about 20 microliters (e.g., about 1, 2, 3, 4, 5, 6, 7, 8, 9, 10, 11, 12, 13, 14, 15, 16, 17, 18, 19, or 20) microliters. In other embodiments, the volume of the suspension, solution, dispersion, or other composition to be administered is at least about 20 to about 100 microliters (e.g., about 20, 25, 30, 35, 40, 45, 50, 55, 60, 65, 70, 75, 80, 85, 90, 95, 100, or more microliters).

Similarly, the number of doses to be administered, when the therapeutically effective amount of immature astrocytes is to spread out over multiple doses, will depend on the total number of immature astrocytes to be administered and the desired concentration or volume. Any number of doses can be administered. In some embodiments, 1, 2, 3, 4, 5, 6, 7, 8, 9, 10, 11, 12, 13, 14, 15, 16, 17, 18, 19, 20, or more doses are to be administered. The doses can be given simultaneously (e.g., administration is by different routes and/or to different sites (e.g., to multiple sites in the brain, multiple sites in the spinal cord, or to one ore more sites in the brain and spinal cord) at the same time), or can be given sequentially. Sequential doses can be administered by different routes and/or to different sites (e.g., to multiple sites in the brain, multiple sites in the spinal cord, or to one or more sites in the brain and spinal cord). Sequential doses can be separated by time interval(s) that one of skill in the art will readily be able to determine. Sequential doses can be separated by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes. Alternatively, sequential doses can be separated by about 1, 2, 3, 4, 5, 6, 7, 8, 9, 10, 11, 12, 13, 14, 15, 16, 17, 18, 19, 20, 21, 22, 23, 24, or more hours. Sequential doses can also be separated by about 1, 2, 3, 4, 5, 6, 7, or more days, or about 1, 2, 3, 4, or more weeks. Alternatively, sequential doses can be separated by about 1, 2, 3, 4, 5, 6, 7, 8, 9, 10, 11, 12, or more months, or about 1, 2, 3, 4, 5 or more years.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

In some embodiments, the therapeutically effective dose may further comprise other components, for example, immunosuppression agents or anti-allergy drugs such as antihistamines, steroids, bronchodilators, leukotriene stabilizers and mast cell stabilizers. Suitable immunosuppression and anti-allergy drugs are well known in the art.

A practitioner of skill in the art will readily be able to discern where and by what route(s) the immature astrocytes are to be administered. Such a determination depends on factors including, but not limited to, the nature and/or severity of the demyelinating disease. In some embodiments, immature astrocytes are delivered to the brain. In particular embodiments, the immature astrocytes are delivered to one or more localized regions of the brain. In other embodiments, the immature astrocytes are delivered to the spinal cord. In particular embodiments, the immature astrocytes are delivered to one or more localized regions of the spinal cord. In some other embodiments, the immature astrocytes are delivered to one or more localized regions of the brain and the spinal cord.

When immature astrocytes are to be directly transplanted or injected into the brain and/or spinal cord, in some embodiments, the immature astrocytes are injected with the aid of a microsyringe and/or microneedle. As a non-limiting example, Hamilton Microliter syringes and needles can be used to inject immature astrocytes. Injection can be performed manually, or under the control of a pump. In some embodiments, the pump is controlled by a computer, which can be used to program various parameters such as the volume to be administered, the rate of administration (e.g., the number of microliters to be administered per minute), etc.

### D. Sources and Identification of Immature Astrocytes

In some embodiments, immature astrocytes administered according to methods of the present invention are derived from precursor cells. As a non-limiting example, immature astrocytes can be derived from pluripotent cells. A pluripotent stem cell can be from any species, so long as it can give rise to immature astrocytes that are functionally compatible and able to survive administration (e.g., transplantation) into the subject recipient. When the subject is a human, the pluripotent stem cell is preferably human. In some instances, the pluripotent stem cell is an embryonic stem cell.

In other embodiments, the pluripotent stem cell is an induced pluripotent stem cell (iPSC). When it is desirable to maximize histocompatibility, iPSCs can be derived from the subject to which the immature astrocytes are to be administered according to methods of the present invention. Any number of adult somatic cells can be reprogrammed to generate iPSCs. Non-limiting examples of suitable cells that can be reprogrammed are fibroblasts (e.g., skin fibroblasts), keratinocytes, liver cells, stomach cells, and neural stem cells. One of skill in the art will be able to determine the appropriate type of adult somatic cell to use for reprogramming, taking into account various consideration (e.g., reprogramming efficiency).

In particular embodiments, iPSCs are generated from cells obtained from umbilical cord blood. Depending on the source of the iPSCs, and ultimately the immature astrocytes, it may be necessary to administer other agents to the subject, such as immunosuppression drugs. One of skill in the art will readily be able to determine the appropriate co-therapy.

Adult somatic cells can be reprogrammed by introducing, for example, proteins, small molecules, and/or nucleic acids that encode proteins such as transcription factors. As a non-limiting example, reprogramming can be achieved by transducing somatic cells with nucleic acids encoding the transcription factors Oct4, Sox2, Klf4, and c-myc, which are known to be involved in the maintenance of pluripotency. Alternatively, nucleic acids encoding the transcription factors Oct4, Sox2, Nanog, and Lin28 can be introduced into the cell to be reprogrammed. As another non-limiting example, reprogramming can be achieved by transducing the somatic cell with a nucleic acid encoding the nuclear receptor Esrrb, together with nucleic acids encoding Oct4 and Sox2. In some cases, factors such as vitamin C can be used to increase the efficiency of inducing somatic cells to become pluripotent. In some embodiments, synthetic or recombinant proteins are introduced to the cell to be induced, or the cell to be induced is forced to express recombinant proteins (e.g., engineered or recombinant transcription factors).

A variety of methods are suitable for introducing nucleic acids, proteins, and/or other factors to cells that are to be reprogrammed into iPSCs. As non-limiting examples, cell reprogramming can be achieved by using single cassette vectors with Cre-Lox mediated transgene excision, nonintegrating viruses (e.g., adenovirus, Sendai virus), mRNA infection and/or transfection, PiggyBac mobile genetic elements, minicircle vectors, or episomal plasmids to transduce cells. In addition to the choice of genes, proteins, and/or other factors to be introduced, the efficiency of reprogramming depends on the method of introduction. Other considerations, such as the introduction of inadvertent mutations, may dictate the choice of method.

In some embodiments, the identity of the immature astrocytes is determined or confirmed. In some instances, the identity is determined or confirmed before the immature astrocytes are administered to the subject. This can be achieved, for example, by determining the presence or level of one or more biomarkers. The one or more biomarkers, in some instances, are expressed by the immature astrocytes. The presence or level of the one or more biomarkers can be determined, for example, by detecting the presence, copy number, or sequence of one or more genes (e.g., sequencing genomic DNA or performing FISH analysis), determining the presence or level of mRNA expression of one or more genes (e.g., quantitative PCR, microarray analysis), detecting epigenetic modifications of DNA (e.g., methylation of genomic DNA), detecting the presence or level of protein expression on the surface of a cell or inside a cell (e.g., immunohistochemistry), determining the presence or amount of a secreted protein (e.g., TIMP-1), or a functional assay (e.g., glutamate transport assay, electrophysiological method such as patch clamping).

When antibodies are used to detect the presence or level of one or more biomarkers, labeled secondary antibodies are commonly used to detect antibodies that have bound to the one or more biomarkers. Secondary antibodies bind to the constant or “C” regions of different classes or isotypes of immunoglobulins IgM, IgD, IgG, IgA, and IgE. Usually, a secondary antibody against an IgG constant region is used. Secondary antibodies against the IgG subclasses, for example, IgG1, IgG2, IgG3, and IgG4, also find use in the present methods. Secondary antibodies can be labeled with any directly or indirectly detectable moiety, including a fluorophore (e.g., fluoroscein, phycoerythrin, quantum dot, Luminex bead, fluorescent bead), an enzyme (e.g., peroxidase, alkaline phosphatase), a radioisotope (e.g., ³H, ³²P ¹²⁵I) or a chemiluminescent moiety. Labeling signals can be amplified using a complex of biotin and a biotin binding moiety (e.g., avidin, streptavidin, neutravidin). Fluorescently labeled anti-human IgG antibodies are commercially available from Molecular Probes, Eugene, OR. Enzyme-labeled anti-human IgG antibodies are commercially available from Sigma-Aldrich, St. Louis, Mo. and Chemicon, Temecula, Calif.

The method of detection of the presence or absence, level, or differential presence, of the one or more biomarkers will correspond with the choice of label of the secondary antibody. For example, if the immature astrocytes and/or one or more of the biomarkers are transferred onto a membrane substrate suitable for immunoblotting, the detectable signals (i.e., blots) can be quantified using a digital imager if enzymatic labeling is used or an x-ray film developer if radioisotope labeling is used. In another example, if the immature astrocytes and/or one or more biomarkers are transferred to a multi-well plate, the detectable signals can be quantified using an automated plate reader capable of detecting and quantifying fluorescent, chemiluminescent, and/or colorimetric signals. Such methods of detection are well known in the art.

General immunoassay techniques are well known in the art. Guidance for optimization of parameters can be found in, for example, Wu, Quantitative Immunoassay: A Practical Guide for Assay Establishment, Troubleshooting, and Clinical Application, 2000, AACC Press; Principles and Practice of Immunoassay, Price and Newman, eds., 1997, Groves Dictionaries, Inc.; The Immunoassay Handbook, Wild, ed., 2005, Elsevier Science Ltd.; Ghindilis, Pavlov and Atanassov, Immunoassay Methods and Protocols, 2003, Humana Press; Harlow and Lane, Using Antibodies: A Laboratory Manual, 1998, Cold Spring Harbor Laboratory Press; and Immunoassay Automation: An Updated Guide to Systems, Chan, ed., 1996, Academic Press.

In certain embodiments, the presence or increased presence of the one or more biomarkers is indicated by a detectable signal (e.g., a blot, fluorescence, chemiluminescence, color, radioactivity) in an immunoassay. This detectable signal (i.e., in a test sample) can be compared to the signal from a control sample or to a threshold value. In some embodiments, the control sample comprises a mature astrocyte. Alternatively, the control sample can comprise a precursor cell (e.g., pluripotent stem cell such as an iPSC or ESC). In some embodiments, the presence or an increased level of a biomarker is indicated when the detectable signal is at least about 10%, 20%, 30%, 50%, 75%, or more greater in comparison to the signal in the control sample or the predetermined threshold value. In some embodiments, the presence or increased level of a biomarker is indicated when the detectable signal in the test sample is at least about 1-fold, 2-fold, 3-fold, 4-fold, or more greater in comparison to the signal in the control sample or the predetermined threshold value. In some embodiments, the absence or a decreased level of a biomarker is indicated when the detectable signal is at least about 10%, 20%, 30%, 50%, 75%, or more lower in comparison to the signal in the control sample or the predetermined threshold value.

Non-limiting examples of biomarkers that are suitable for identifying astrocytes and/or differentiating immature and mature astrocytes include tissue inhibitor of metalloproteinase-1 (TIMP-1), glial fibrillary acidic protein (GFAP), S100 calcium-binding protein B (S100β), CD44, vimentin, nuclear factor 1 A-type (NF1A), excitatory amino acid transporter 1 (EAAT1), and combinations thereof. In some embodiments, the level of TIMP-1, CD44, vimentin, NF1A, and/or GFAP is higher in a test sample than in the control sample or predetermined threshold value, wherein the control sample comprises a mature astrocyte, indicating that the test sample comprises an immature astrocyte. In other embodiments, the level of EAAT1 is lower in a test sample than in the control sample or predetermined threshold value, wherein the control sample comprises a mature astrocyte, indicating that the test sample comprises an immature astrocyte.

### E. Kits

In another aspect, the invention provides a kit for preventing a demyelinating disease in a subject, for treating a demyelinating disease in a subject, or for reducing demyelination, inducing remyelination, promoting oligodendroglial progenitor cell (OPC) proliferation, and/or promoting oligodendrocyte differentiation in a subject. In some embodiments, the kit comprises an immature astrocyte or a plurality thereof, a composition, and/or a pharmaceutical composition of the present invention described herein. The kits are useful for preventing or treating any demyelinating disease, some non-limiting examples of which include periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof.

Materials and reagents to carry out the various methods of the present invention can be provided in kits to facilitate execution of the methods. As used herein, the term “kit” includes a combination of articles that facilitates a process, assay, analysis, or manipulation. In particular, kits of the present invention find utility in a wide range of applications including, for example, diagnostics, prognostics, therapy, and the like.

Kits can contain chemical reagents as well as other components. In addition, the kits of the present invention can include, without limitation, instructions to the kit user, apparatus and reagents for sample collection and/or purification, apparatus and reagents for product collection and/or purification, apparatus and reagents for administering immature astrocytes or other composition(s) of the present invention, apparatus and reagents for determining the level(s) of biomarker(s), sample tubes, holders, trays, racks, dishes, plates, solutions, buffers or other chemical reagents, suitable samples to be used for standardization, normalization, and/or control samples. Kits of the present invention can also be packaged for convenient storage and safe shipping, for example, in a box having a lid.

In some embodiments, the kits also contain control samples for detecting the presence or level of one or more biomarkers. In some embodiments, the control samples comprise mature astrocytes and/or precursors of immature astrocytes. In some embodiments, the kits contain samples for the preparation of a titrated curve of one or more biomarkers in a sample, to assist in the evaluation of quantified levels of biomarkers in a biological sample (e.g., a test cell).

### IV. Examples

The present invention will be described in greater detail by way of a specific example. The following example is offered for illustrative purposes only, and is not intended to limit the invention in any manner. Those of skill in the art will readily recognize a variety of noncritical parameters which can be changed or modified to yield essentially the same results.

### Example 1 Human iPSC-derived Immature Astroglia Promote Oligodendrogenesis

### Summary

Astrocytes, once considered passive support cells, are increasingly appreciated as dynamic regulators of neuronal development and function, in part via secreted factors. The extent to which they similarly regulate oligodendrocytes, or proliferation and differentiation of oligodendrocyte progenitor cells (OPCs) is less well understood. In this example, astrocytes were generated from human pluripotent stem cells (hiPSC-Astros) and it was demonstrated that immature astrocytes—as opposed to mature astrocytes—promoted oligodendrogenesis in vitro. In the periventricular leukomalacia (PVL) mouse model of neonatal hypoxic/ischemic encephalopathy, associated with cerebral palsy in humans, transplanted immature hiPSC-Astros promotes myelinogenesis and behavioral outcome. Furthermore TIMP-1 was identified as a selectively upregulated component secreted from immature hiPSC-Astros. In addition, in the rat PVL model, intranasal administration of conditioned medium from immature hiPSC-Astros promoted oligodendrocyte maturation in a TIMP-1 dependent manner. The experimental results presented in this example demonstrate stage-specific developmental interactions between astroglia and oligodendroglia, with important therapeutic implications for promoting myelinogenesis.

### Introduction

Astrocytes play important roles in organizing and maintaining brain structure and function (Barres, 2008). Astrocytes go through prenatal and protracted postnatal maturation during development and can undergo a spectrum of functional changes associated with development (Molofsky et al., 2012; Pekny and Pekna, 2014), serving stage-specific roles in assisting neuronal development, such as synapse stabilization and elimination (Chung et al., 2013; Molofsky et al., 2012). However, it is unclear how astrocytes, at specific immature and mature stages, may differently regulate development of oligodendrocytes, myelin-producing cells in the CNS.

Human pluripotent stem cells (hPSCs), including human embryonic stem cells (hESCs) and induced pluripotent stem cells (iPSCs), have been efficiently differentiated to astrocytes (Emdad et al., 2011; Jiang et al., 2013b; Krencik et al., 2011; Roybon et al., 2013; Shaltouki et al., 2013). The progenies differentiated from hPSCs are reflective of very early human development (<6 weeks) (Patterson et al., 2012). Particularly, hPSC-derived astrocytes differentiated by using chemically defined, xeno-free protocols can be maintained at an immature stage in culture (Chen et al., 2014a; Emdad et al., 2011; Jiang et al., 2013b; Krencik et al., 2011; Shaltouki et al., 2013). Moreover, hPSC-derived immature astrocytes can be further differentiated to astrocytes with defined mature phenotypes (Krencik et al., 2011; Roybon et al., 2013). Thus, astroglia derived from hPSCs provide an unprecedented opportunity to investigate the interaction between oligodendroglia and human astrocytes that are at defined immature and mature stages.

Astrocytes influence myelination in various myelin loss disorders. Prior studies demonstrated that oligodendrocytes preferentially remyelinate axons in areas containing astrocytes (Franklin et al., 1991; Talbott et al., 2005). However, astroglia-based therapy for myelin loss disorders has been less studied (Chen et al., 2015), because most of the disorders are associated with profound astrocyte activation and formation of glial scar (Pekny and Pekna, 2014). Scarring astrocytes are regarded as a barrier to regeneration, partly due to secretion of factors that halt survival and differentiation of oligodendroglia progenitor cells (OPCs) (Back et al., 2005; Nash et al., 2011). Recent studies have also suggested that in the acute phase of injuries, astrogliosis is a defensive reaction. Reactive astrocytes recapitulate numerous processes that are involved in the early development of immature astroglia and exhibit positive effects in the acute phase of injuries (Pekny and Pekna, 2014), but reactivated processes often go awry later, leading to the detrimental effects of the astrocytes on regeneration (Gallo and Deneen, 2014). Recent studies (Jiang et al., 2013b; Noble et al., 2011) have demonstrated that transplanted immature astrocytes do not become reactive after CNS injury. Immature but not mature astrocytes are neuroprotective and suppress the activation of endogenous astrocytes and glial scar formation (Chen et al., 2015). Thus, there is a strong rationale to examine whether transplantation of hPSC-derived astrocytes at a defined immature stage could regulate differentiation of endogenous OPCs and promote myelinogenesis.

Perinatally-acquired white matter injury, induced by perinatal hypoxia-ischemia and referred to as periventricular leukomalacia (PVL), is the most common cause of brain injury in premature infants. PVL is the leading cause of cerebral palsy and long-term neurological morbidity (Deng, 2010). Currently, no effective treatment exists. One of the major causes of the white matter injury is that pre-myelinating oligodendrocytes are particularly vulnerable in PVL (Haynes et al., 2003). The decrease of pre-myelinating oligodendrocytes leads to an upstream increase of OPCs, but these OPCs are arrested at the progenitor stage and fail to efficiently differentiate into myelin-producing oligodendrocytes (Fancy et al., 2011; Jablonska et al., 2012; Reid et al., 2012; Segovia et al., 2008). This work demonstrates that immature hiPSC-derived astrocytes (“hiPSC-Astros”), but not mature hiPSC-Astros and mature astrocytes isolated from the human brain, strongly promote proliferation of OPCs and differentiation of OPCs to oligodendrocytes. Moreover, immature hiPSC-Astros promote myelination and recovery of behavioral performance in animal models of PVL injury. Mechanistically, we show that immature hiPSC-Astros regulate OPC differentiation via secreted molecules including tissue inhibitor of metalloproteinase-1 (TIMP-1) both in vitro and in vivo.

### Results

Generation of Immature and Mature hiPSC-Astros

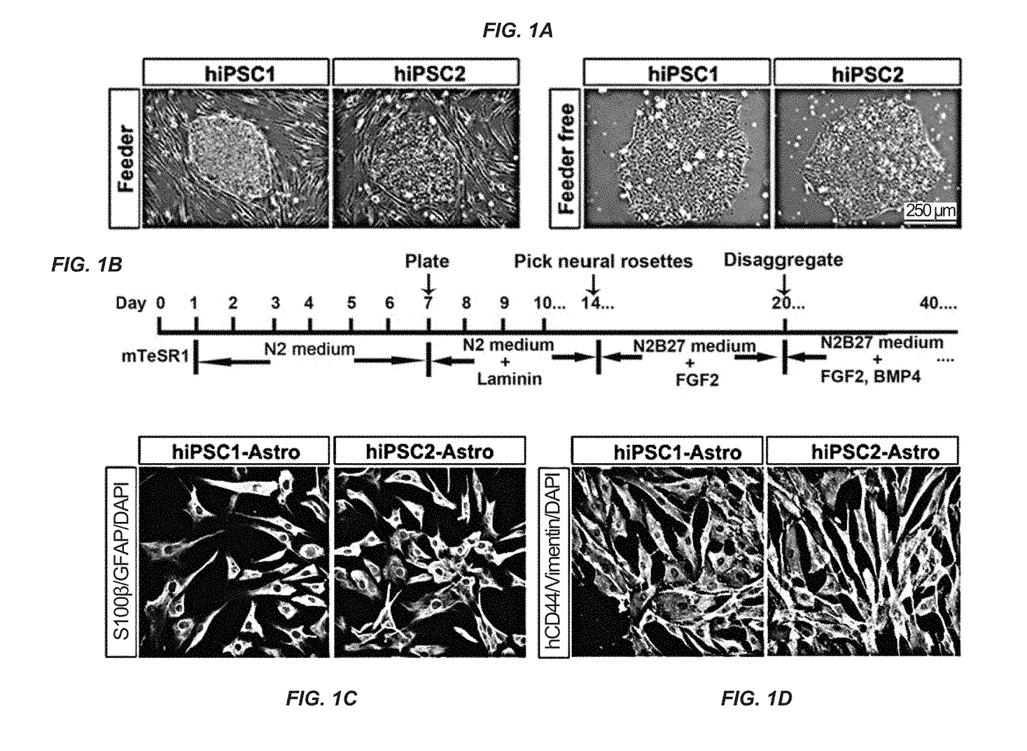

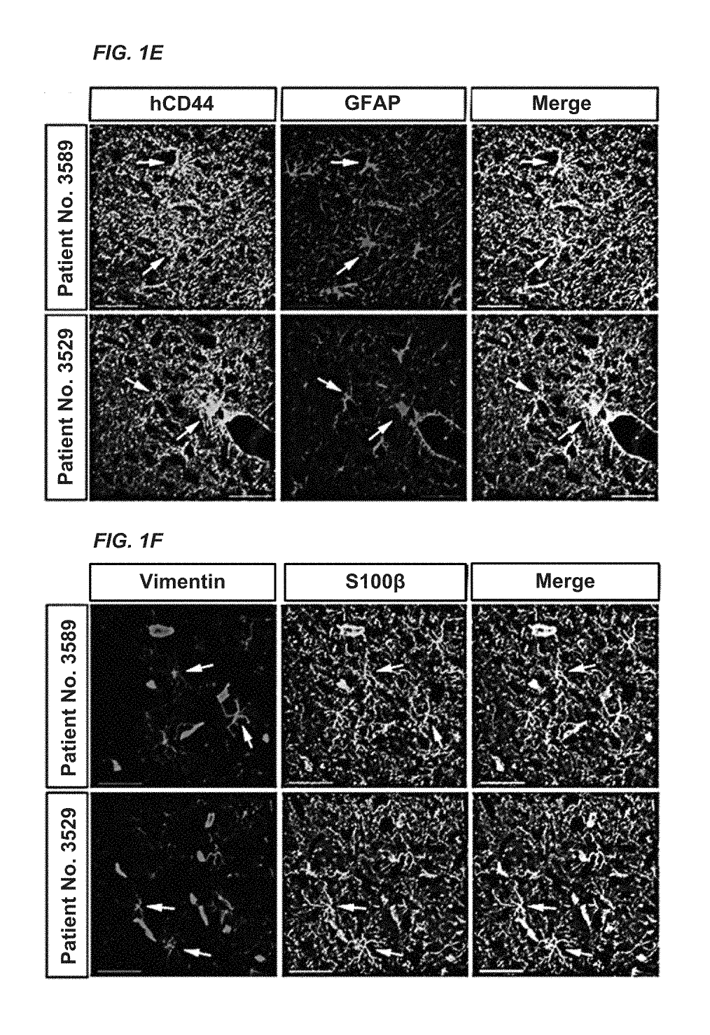

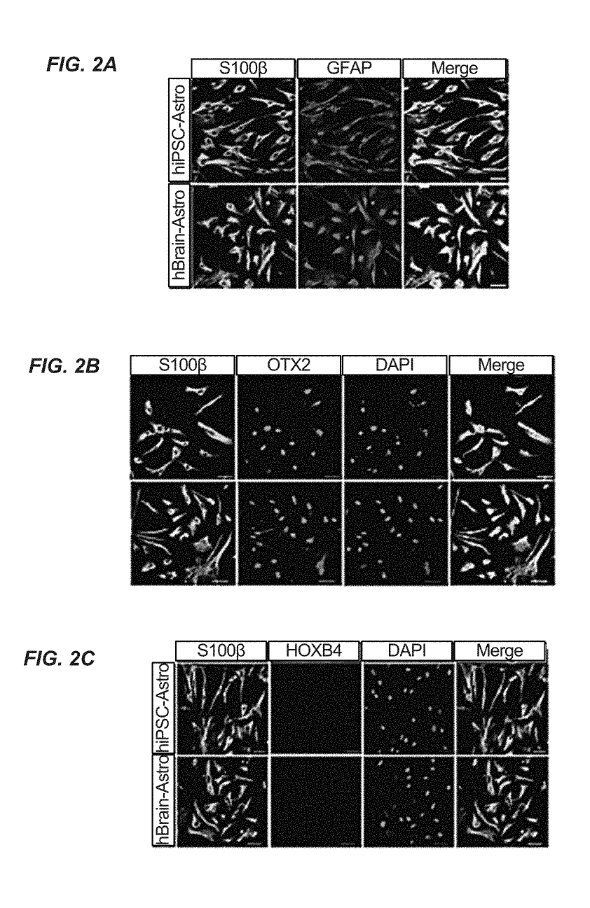

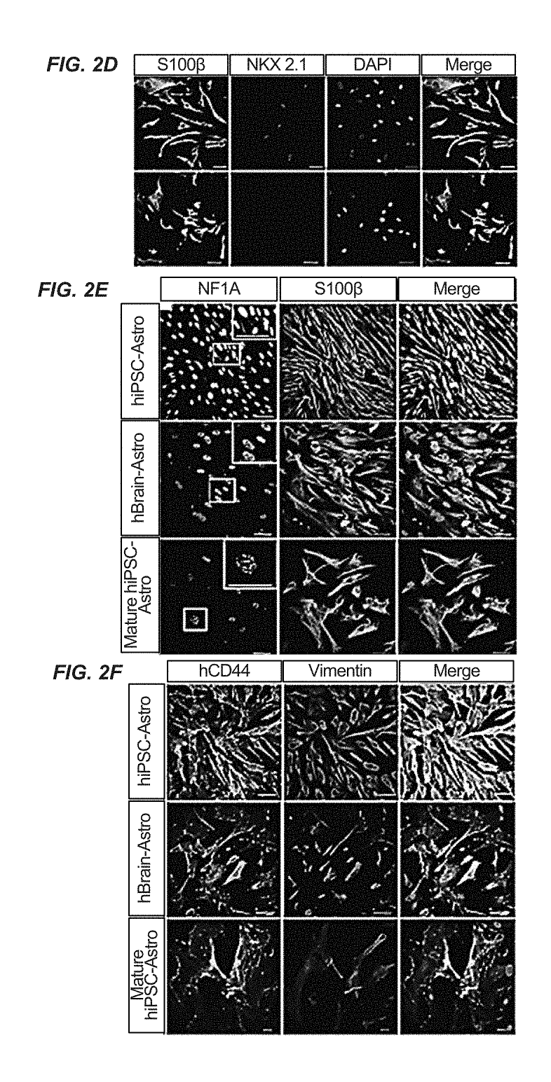

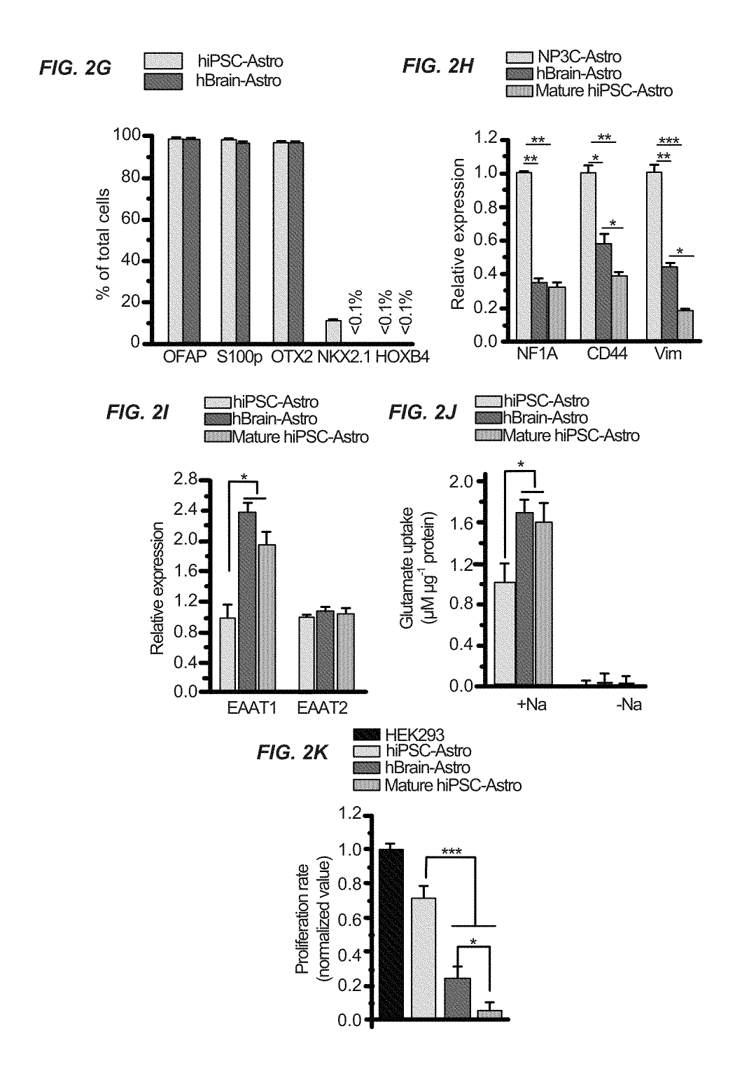

By using an established protocol (Jiang et al., 2013b), astroglia were derived from two hiPSC lines generated from healthy individuals (Chen et al., 2014a) (FIGS. 1A and 1B). These hiPSC-Astros expressed astroglial markers glial fibrillary acidic protein (GFAP) and S100β (FIG. 1C). The regional identities of the hiPSC-Astros were then compared with human brain-derived astrocytes (hBrain-Astros) isolated from the cerebral cortex of human brain. Similar to hiPSC-Astros, hBrain-Astros expressed astroglial markers GFAP and S100β (FIG. 2A). The hBrain-Astros mainly expressed the mid/forebrain marker OTX2 (FIGS. 2B and 2G) but not the hindbrain/spinal cord-specific marker HOXB4 (FIGS. 2C and 2G). There were nearly no hBrain-Astros that expressed the ventral marker NKX2.1 (<0.1%, FIGS. 2D and 2G). Similarly, the vast majority of the hiPSC-Astros also showed mid/forebrain identity, as indicated by expressing OTX2 (FIGS. 2B and 2G), but not HOXB4 (<0.1%, FIGS. 2C and 2G). A small percent of hiPSC-Astros weakly expressed NKX2.1 (FIGS. 2C and 2G).

Nearly all the hiPSC-Astros also expressed human CD44 (hCD44) and vimentin (FIG. 1D), indicating that these hiPSC-Astros were immature (Liu et al., 2004) (Dahl et al., 1981; Jiang et al., 2013b). We then induced immature hiPSC-Astros to become mature astrocytes by treatment with fibroblast growth factor 1 (FGF1), leukemia inhibitory factor (LIF) and ciliary neurotrophic factor (CNTF) (Krencik et al., 2011; Roybon et al., 2013). After 30- to 50-day treatment, expression of an astroglial maturation marker, nuclear factor-1A (NF1A) (Deneen et al., 2006; Roybon et al., 2013), was examined in order to verify the mature phenotypes of these human astrocytes. The expression of NF1A was abundant in the immature hiPSC-Astros, but markedly reduced in the hBrain-Astros and mature hiPSC-Astros (FIG. 2E). Quantitative PCR results showed that the immature hiPSC-Astros had a significantly higher (i.e., about 3-fold higher) expression level of NF1A than hBrain-Astros and mature hiPSC-Astros (FIG. 2H). The expression of hCD44 and vimentin was subsequently examined in these human astrocytes. Nearly all of the hiPSC-Astros, hBrain-Astros, and mature hiPSC-Astros were positive for hCD44 and vimentin staining (FIG. 2F). However, qPCR results indicated a significantly higher expression level of hCD44 in the immature hiPSC-Astros, compared to hBrain-Astros (i.e., about 1.7-fold higher) and mature hiPSC-Astros (i.e., about 2.6-fold higher) (FIG. 2H). Moreover, qPCR results also showed that immature hiPSC-Astro expressed the highest level of vimentin, which was about 2.2-fold and 5.3-fold higher than hBrain-Astros and mature hiPSC-Astros, respectively (FIG. 2H). The expression levels of hCD44 and vimentin were higher in hBrain-Astros than those in mature hiPSC-Astros (FIG. 2H). It was further observed that in the human brain tissues derived from normal patients at the age of less than 6 months, human immature astrocytes in situ labeled by GFAP or S100β also expressed hCD44 and vimentin (FIGS. 1E and 1F).

Next, expression of mRNAs encoding the astrocyte-specific glutamate transporters excitatory amino acid transporter 1 and 2 (EAAT1 and 2) were quantified in all the astroglial preparations. Consistent with a previous study (Roybon et al., 2013), it was found that EAAT1 was expressed at a higher level in mature hiPSC-Astros (i.e., about 1.9-fold higher) and hBrain-Astros (i.e., about 2.3-fold higher) than in immature hiPSC-Astros (FIG. 21), while EAAT2 level was not significantly different. The hBrain-Astros expressed EAAT1 and 2 at levels similar to mature hiPSC-Astros. In agreement with the expression of glutamate transporters, hBrain-Astros and mature hiPSC-Astros exhibited about a 1.6-fold increase in sodium-dependent glutamate transport activity, compared to immature hiPSC-Astros (FIG. 2J). In addition, it is generally accepted that mature astrocytes are not proliferative. We also observed significantly lower proliferation rate of mature hiPSC-Astros and hBrain-Astros than that of immature hiPSC-Astros (FIG. 2K). Together, these data demonstrate that the hiPSC-Astros in this example represented human astrocytes with immature phenotypes, whereas hBrain-Astros and the hiPSC-Astros treated with FGF1 represented human astrocytes with mature phenotypes.

Immature hi-PSC-Astros Promoted Proliferation of OPCs

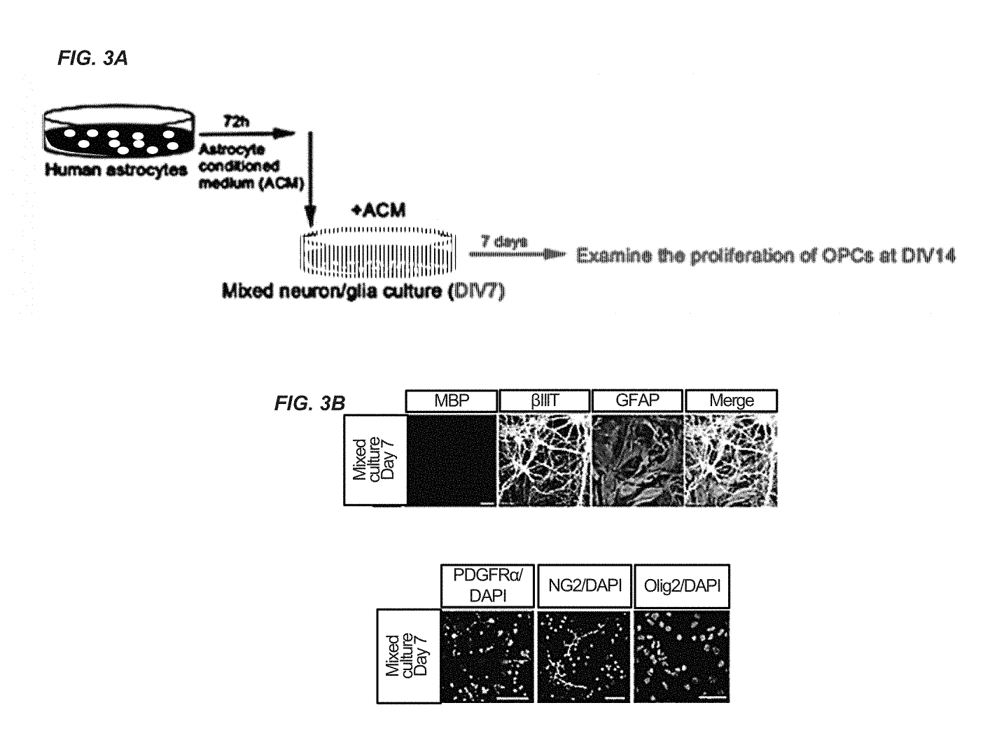

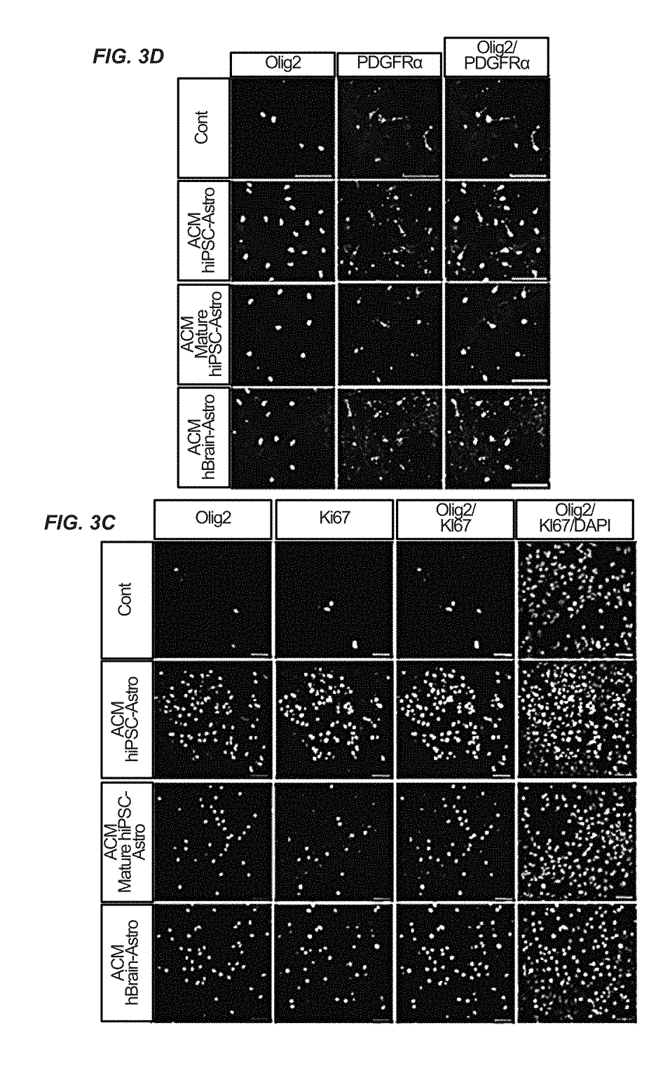

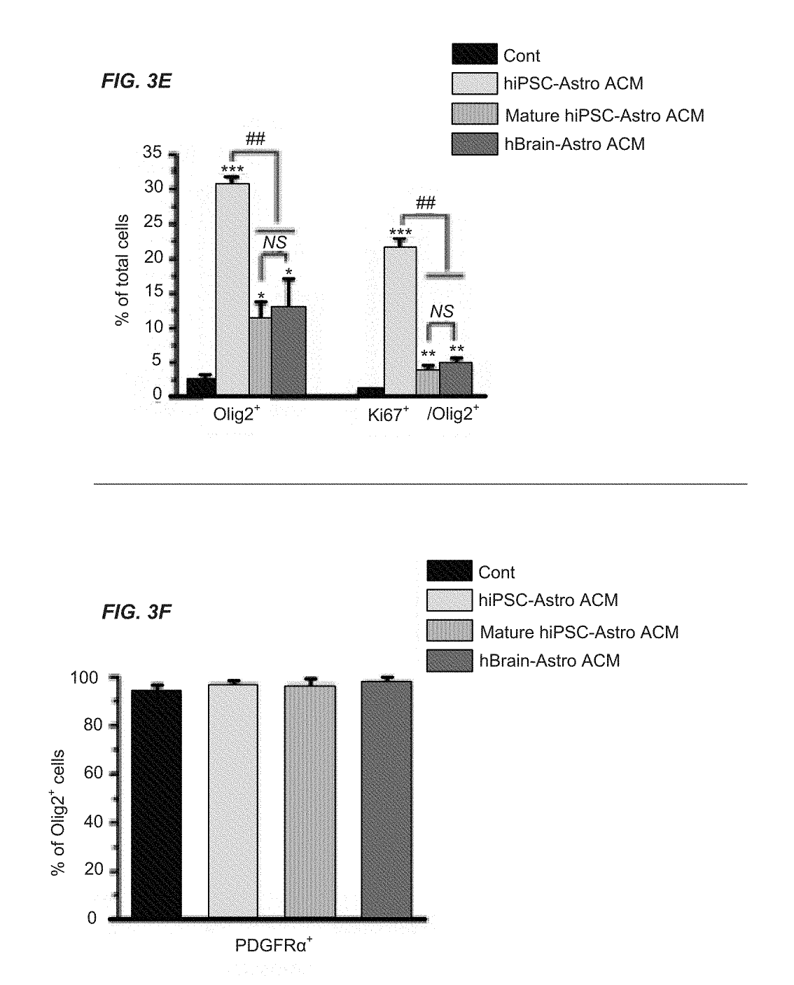

To develop astroglia-based cell therapy for myelin loss disorders, it was first investigated how the astroglia differentiated from hiPSCs interacted with oligodendroglia, particularly in the presence of neurons. To this end, a primary mixed neuron/glia culture was fed at 7 days in vitro (DIV) with astrocyte-conditioned medium (ACM) collected from immature hiPSC-Astros (hiPSC-Astro ACM), from hBrain-Astros (hBrain-Astro ACM) or from mature hiPSC-Astro (mature hiPSC-Astro ACM). The population of oligodendroglial lineage cells in the culture was then examined at DIV 14 (FIG. 3A). At DIV 7, there were βIIITubulin⁺ neurons, GFAP⁺ astrocytes and OPCs labeled by NG2⁺, platelet-derived growth factor receptor alpha (PDGFRα), or Olig2, but no MBP⁺ mature oligodendrocytes (FIG. 3B). At DIV 14, oligodendroglial lineage cells identified by Olig2 staining were found in control group (FIGS. 3C and 3E), but few of them were proliferating as indicated by not expressing Ki67 (FIGS. 3C and 3E). Compared to control group, more Olig2⁺ cells and Olig2⁺/Ki67⁺ proliferating cells were identified in the group treated with mature hiPSC-Astro ACM and in the group treated with hBrain-Astro ACM (FIGS. 3C and 3E). Notably, compared to mature hiPSC-Astro ACM and hBrain-Astro ACM groups, the group treated with hiPSC-Astro ACM had much more Olig2⁺ cells and Olig2⁺/Ki67⁺ proliferating cells (FIGS. 3C and 3E). For all groups, the vast majority (i.e., more than 93.0%) of the Olig2⁺ cells were labeled by PDGFRα, indicating their nature of OPCs (FIGS. 3D and 3F). Hence, human astrocytes promoted proliferation of endogenous OPCs in the primary culture. Immature hiPSC-Astros had a much stronger capacity in promoting OPC proliferation than mature hiPSC-Astros and hBrain-Astros.

Immature hiPSC-Astros Boosted Differentiation of OPCs to Oligodendrocytes

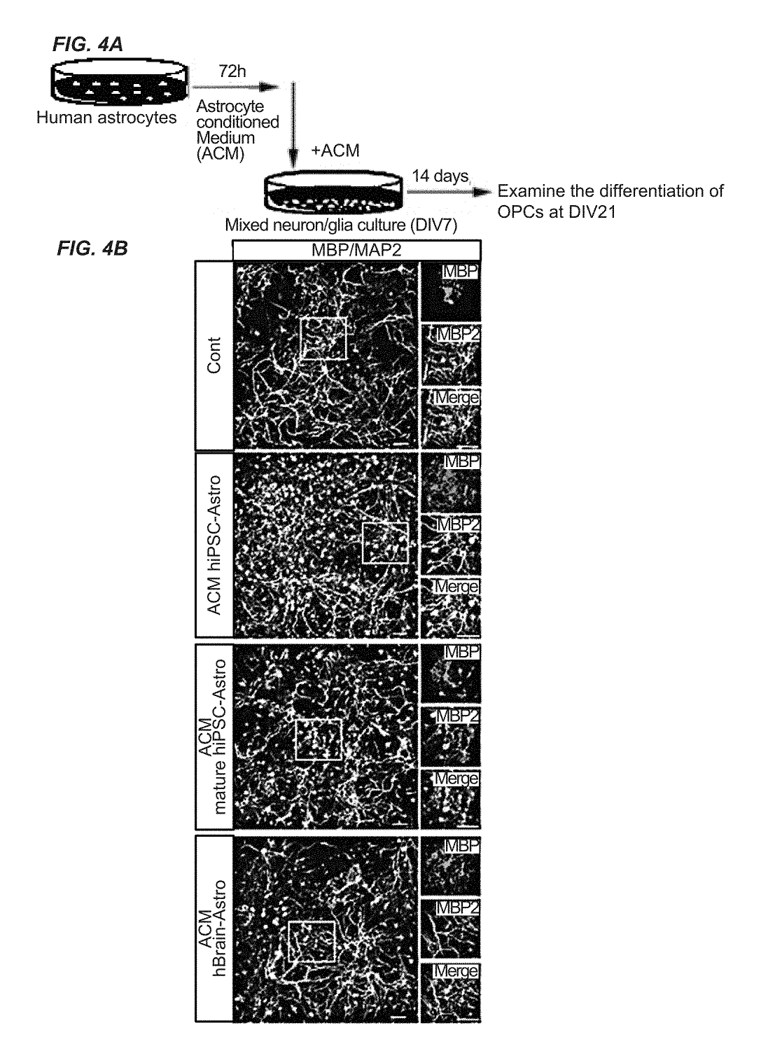

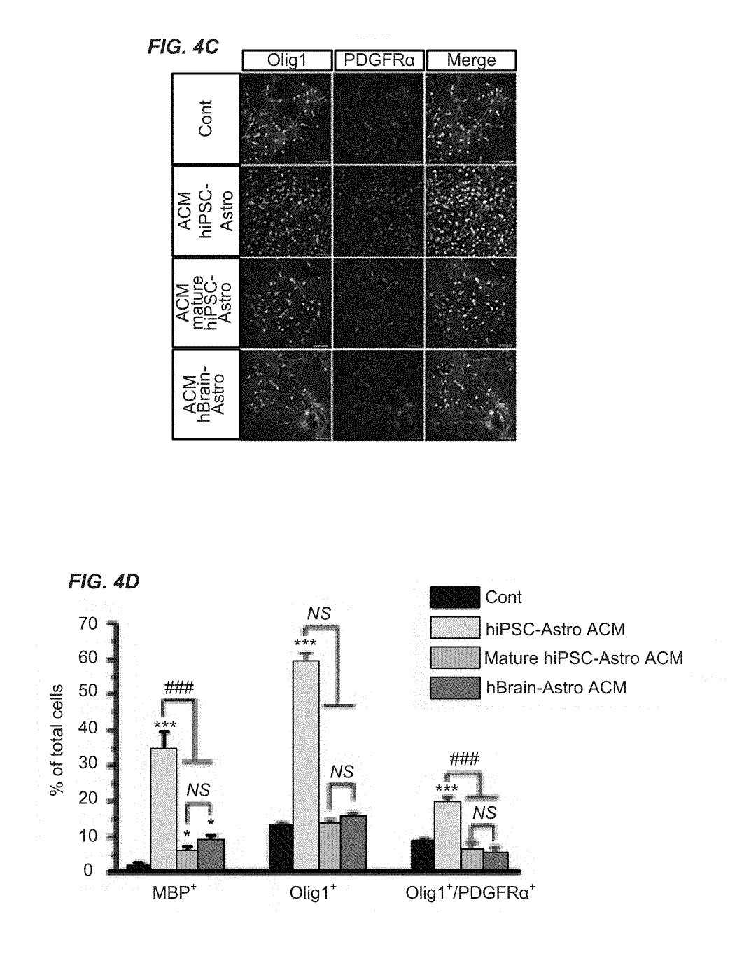

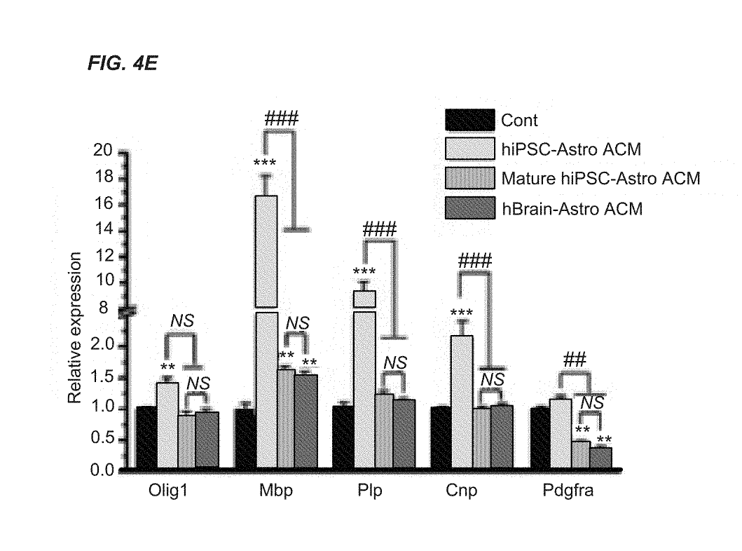

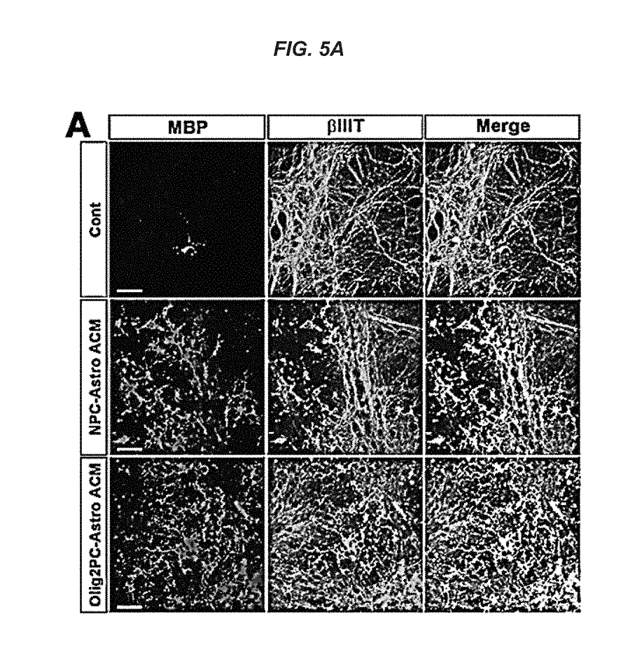

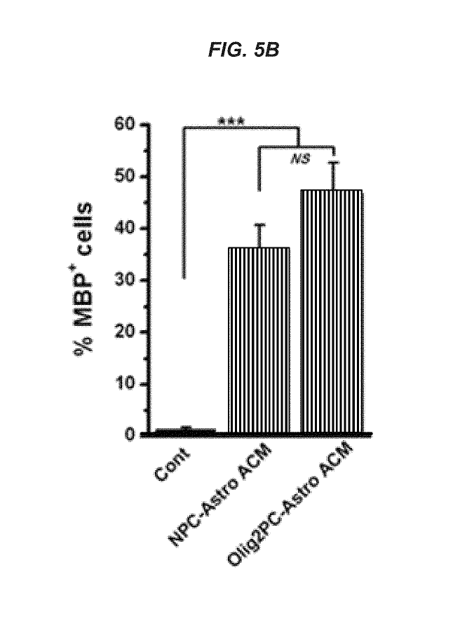

The population of MBP⁺ mature oligodendrocytes at 14 days (DIV 21) after addition of ACM (FIG. 4A). Olig1 was used to label the oligodendroglial cells in the cultures, because Olig2 expression might have down-regulated as the OPCs initiated the myelination program (Jiang et al., 2013a). It was found that in the control group, there were few MBP⁺ cells (FIGS. 4B and 4D), and the majority of Olig1⁺ oligodendroglia expressed PDGFRα (FIG. 4C), indicating that OPCs in control culture did not robustly differentiate to oligodendrocytes and were stuck in the progenitor cell stage. Compared to the control group, there were more MBP⁺ cells in the mature hiPSC-Astro ACM and hBrain-Astro ACM groups. Strikingly, large numbers of MBP⁺ oligodendrocytes were found in the hiPSC-Astro ACM group, suggesting that hiPSC-Astro ACM promoted differentiation of OPCs to oligodendrocytes. A previous study (Jiang et al., 2013b) reported the generation of two subtypes of human astrocytes from hESCs, NPC-Astros and Olig2PC-Astros. Here, it was observed that ACM collected from both of these two subtypes of hESC-derived immature astrocytes also strongly promoted OPC differentiation at DIV 21 (FIG. 5), indicating that the effects on OPC differentiation were common to all the immature astrocytes derived from hiPSCs and hESCs.

To examine the population of OPCs in the culture at DIV 21, Olig1 was double stained with PDGFRα. It was found that there were significantly more Olig1⁺ oligodendroglia in the hiPSC-Astro ACM group (FIGS. 4C and 4D), compared to the control group, the mature hiPSC-Astro ACM group, and the hBrain-Astro ACM group. Moreover, significantly more Olig1⁺/PDGFRα⁺ OPCs were found in the hiPSC-Astro ACM group (FIGS. 4C and 4D) than in the control group, the mature hiPSC-Astro ACM group, and the hBrain-Astro ACM group. To further confirm these findings, qPCR was performed to examine the gene expression of Olig1, mature oligodendroglial markers Mbp, Plp, and Cnp, and the OPC marker Pdgfra. It was consistently observed that the hiPSC-Astro ACM group had the highest expression of Olig1 (FIG. 4E). Gene transcripts encoding mature oligodendrocyte markers were also highly expressed in the hiPSC-Astro ACM group. In particular, Mbp expression was 16.6-fold higher in the hiPSC-Astro ACM group than that in the control group. The expression of Plp and Cnp were respectively 9.2-fold and 2.1-fold higher in the hiPSC-Astro ACM group than those in the control group. The expression of Pdgfra was lower (i.e., 0.5-fold) in the mature hiPSC-Astro ACM and hBrain-Astro ACM groups, compared to the control and hiPSC-Astro ACM groups. Altogether, these data indicate that compared to the addition of mature hiPSC-Astro ACM or hBrain-Astros ACM, the addition of hiPSC-Astro ACM not only replenished the OPCs pool by robustly promoting OPC proliferation, but also strongly boosted the differentiation of OPCs to oligodendrocytes.

Immature hiPSC-Astros Regulated OPC Differentiation Partly Via Release of TIMP-1

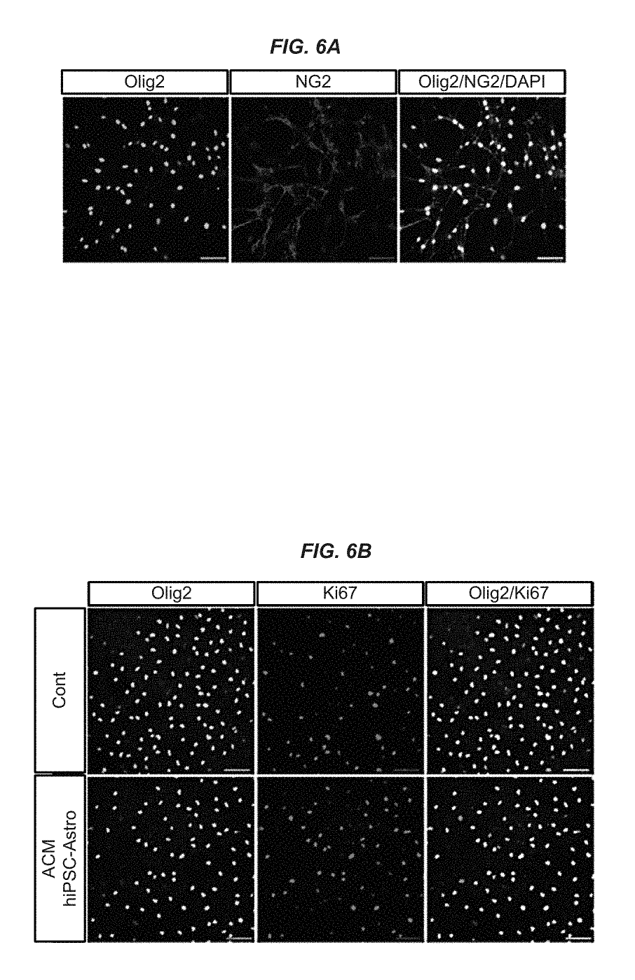

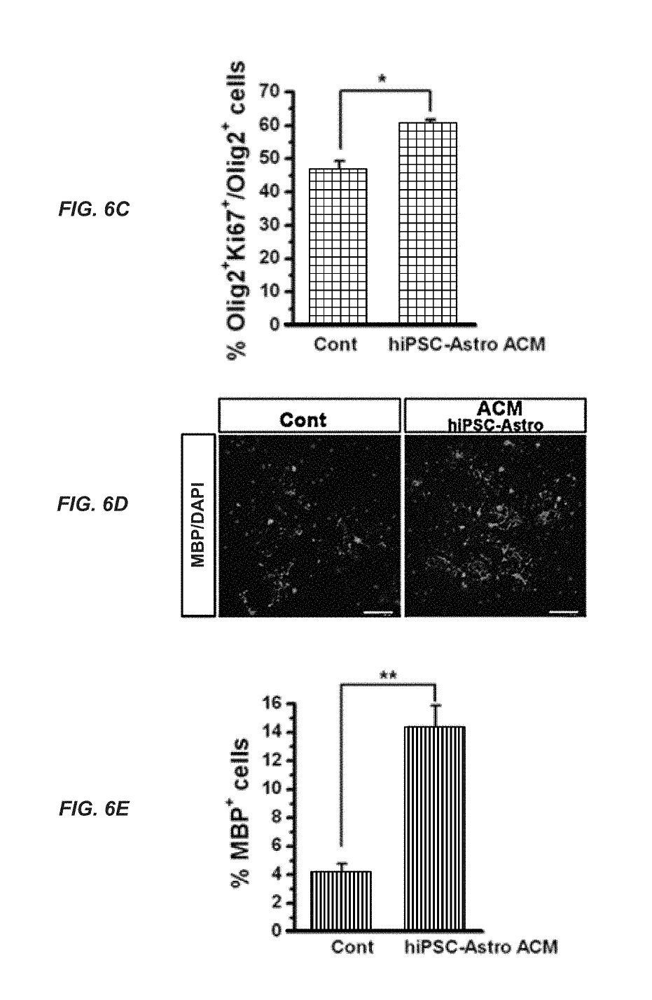

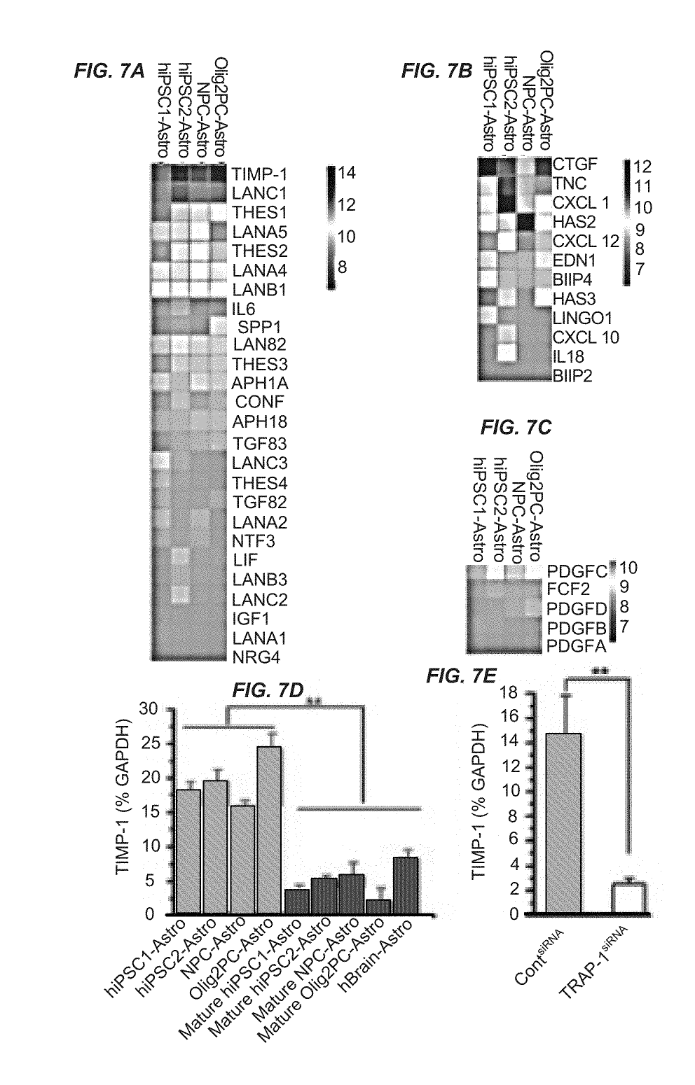

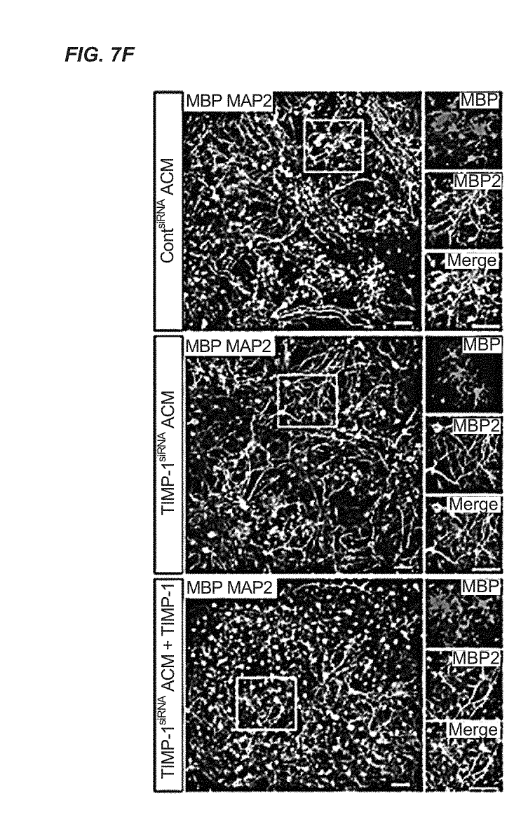

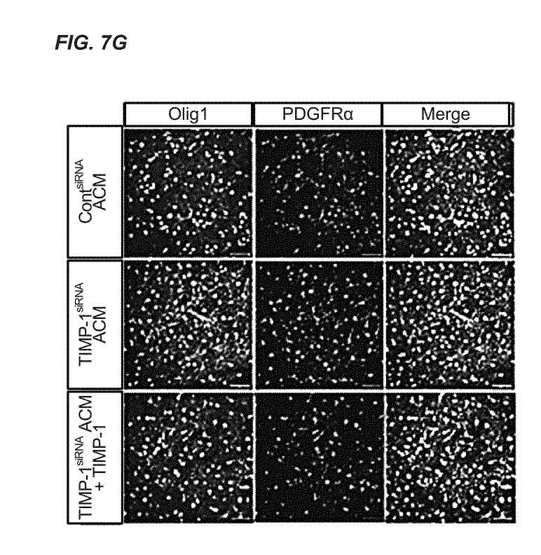

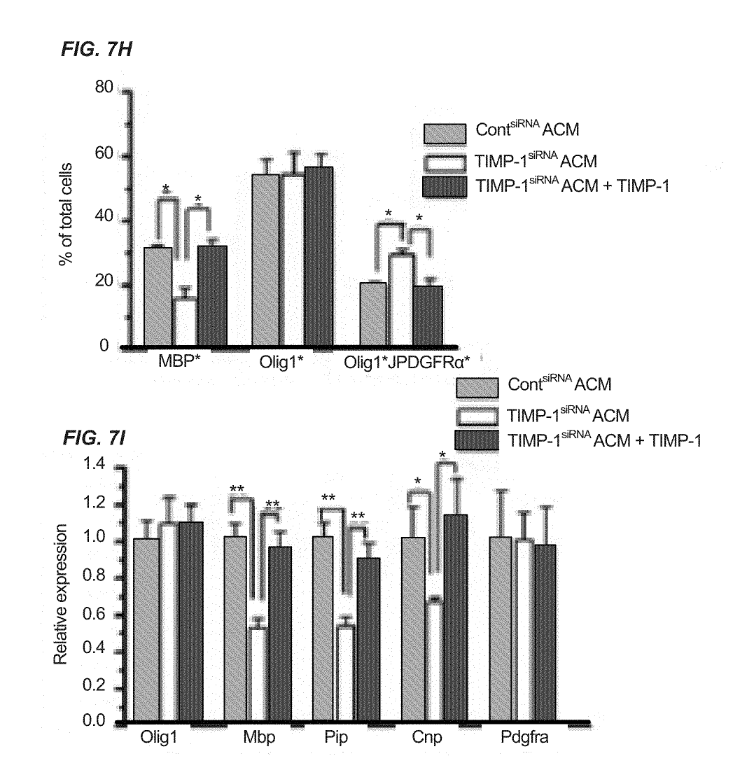

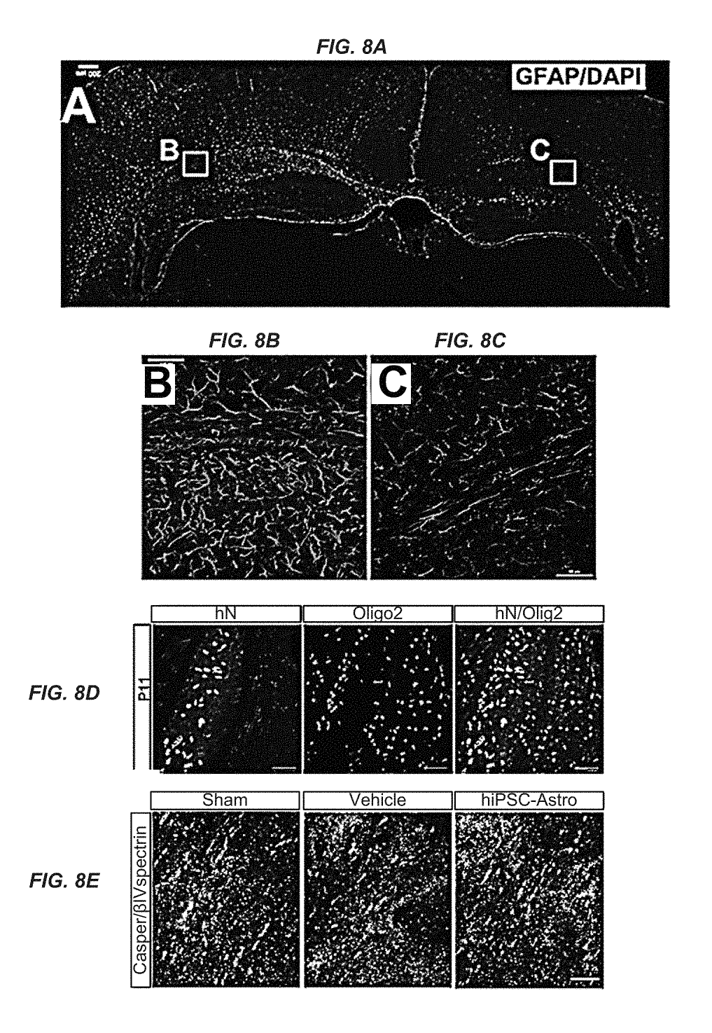

To explore the mechanisms underlying the effects of immature hiPSC-Astros on OPCs, the hiPSC-Astro ACM was added to a purified culture of primary mouse OPCs. It was found that the hiPSC-Astro ACM similarly promoted proliferation and differentiation of OPCs in the purified culture (FIG. 6), suggesting that hiPSC-Astro ACM had direct effects on OPCs. Then, global gene expression was measured by microarray and the data obtained from hiPSC1-Astros, hiPSC2-Astros, and hESC-derived astrocytes, including NPC-Astros and Olig2PC-Astros (Chen et al., 2014a; Jiang et al., 2013b), was analyzed. Analysis was focused on the genes encoding astrocyte-secreted factors that were reported to directly regulate OPC proliferation and differentiation. The detailed information of these genes is shown in Table 1. Notably, immature astrocytes expressed gene transcripts encoding factors that both promote myelination, such as TIMP-1, laminin and TSPs (FIG. 7A), and inhibit myelination, such as CTGF, TnC and CXCL1 (FIG. 7B). Moreover, immature astrocytes expressed gene transcripts encoding PDGF and FGF2 (FIG. 7C), which are OPC mitogens. Therefore, the gene expression of secreted factors with various effects on OPCs may collectively account for the compositive effects of immature astrocytes on OPCs.

Among the gene transcripts encoding factors that promote myelination, it was found that the top highly expressed gene was TIMP-1, which has been previously reported to critically regulate oligodendrocyte development in mice (Moore et al., 2011). Expression of TIMP-1 was then verified in all human astrocytes by qPCR. The result showed that TIMP-1 expression was abundant in immature hiPSC-Astros and hESC-Astros (FIG. 7D), whereas its expression was significantly decreased in mature hiPSC-Astros, mature hESC-Astros, and hBrain-Astros (FIG. 7D). To further investigate the role TIMP-1 in the effects of immature hiPSC-Astros on OPC differentiation, TIMP-1 expression was inhibited in hiPSC-Astros by small interfering RNA (siRNA). qPCR analysis showed that, at 48 hours after transfection of TIMP-1 siRNA, TIMP-1 expression was significantly reduced (i.e., 5.8-fold), compared to hiPSC-Astros transfected with control siRNA (FIG. 7E). Next, the primary mixed neuron/glia culture was fed at DIV7 with conditioned medium collected from hiPSC-Astros transfected with TIMP-1 siRNA (TIMP-1^{siRNA} ACM), control conditioned medium collected from hiPSC-Astros transfected with control siRNA (Cont^{siRNA} ACM), and TIMP-1^{siRNA} ACM supplemented with TIMP-1 (10 ng/mL). Then, OPC differentiation at DIV 21 was examined. There were less MBP⁺ oligodendrocytes in the TIMP-1^{siRNA} ACM group than those in the Cont^{siRNA} ACM group (FIGS. 7F and 7H). Adding TIMP-1 to TIMP-1^{siRNA} ACM increased the percentage of MBP⁺ oligodendrocytes. The total number of Olig1⁺ cells was not significantly different among groups (FIGS. 7G and 7H), suggesting that TIMP-1 knockdown did not change the effects on increasing OPC proliferation. It was also observed that there were more Olig1⁺/PDGFRα⁺ OPCs in the TIMP-1^{siRNA} ACM group, compared to the Cont^{siRNA} and TIMP-1^{siRNA} plus TIMP-1 ACM groups (FIGS. 7G and 7H). The qCPR results further confirmed these observations. Olig1 expression was not significantly different among the three groups (FIGS. 7I). The TIMP-1^{siRNA} ACM group had significantly lower expression of the mature oligodendrocyte genes Mbp (i.e., about 0.5-fold), Plp (i.e., about 0.5-fold), and Cnp (i.e., about 0.6-fold) compared to the Cont^{siRNA} and TIMP-1^{siRNA} plus TIMP-1 ACM groups. Together, these data indicate that TIMP-1 secreted by immature hiPSC-Astros contributed to their effects on promoting OPC differentiation, but not to their effects on increasing OPC proliferation.

Transplanted Immature hiPSC-Astros Promoted Myelination in a Mouse Model of PVL

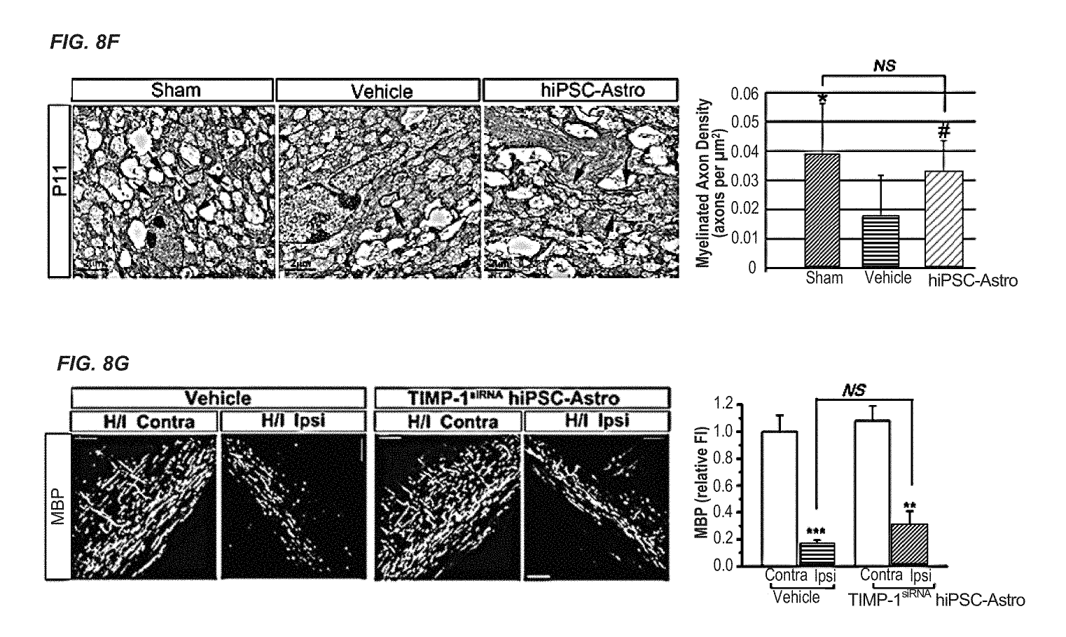

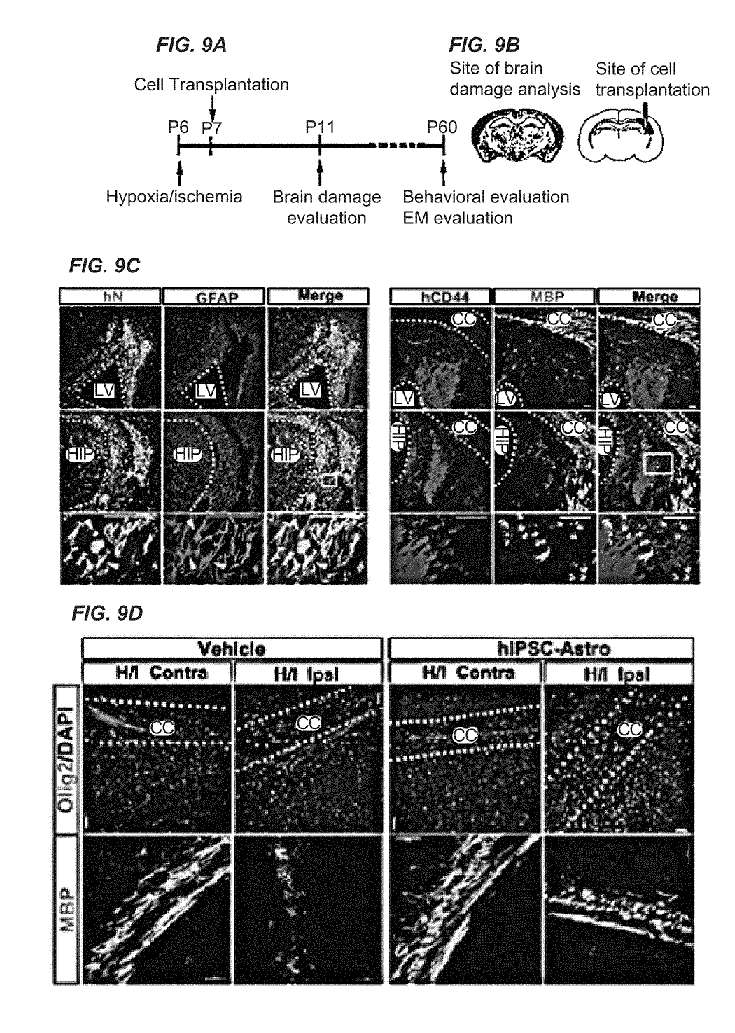

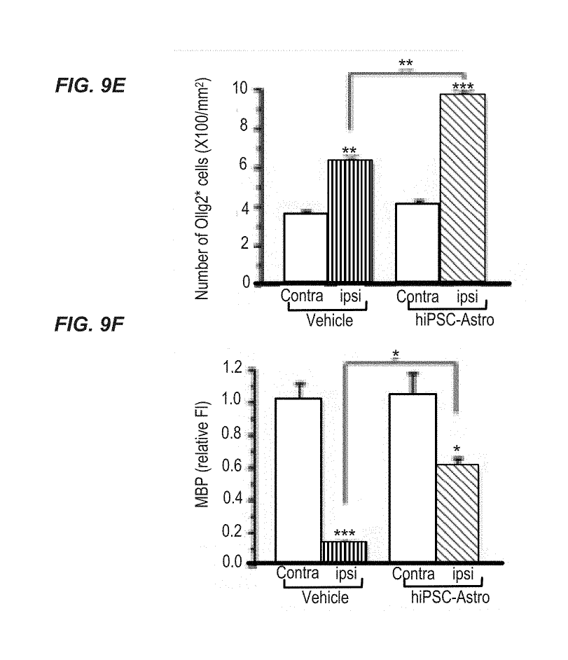

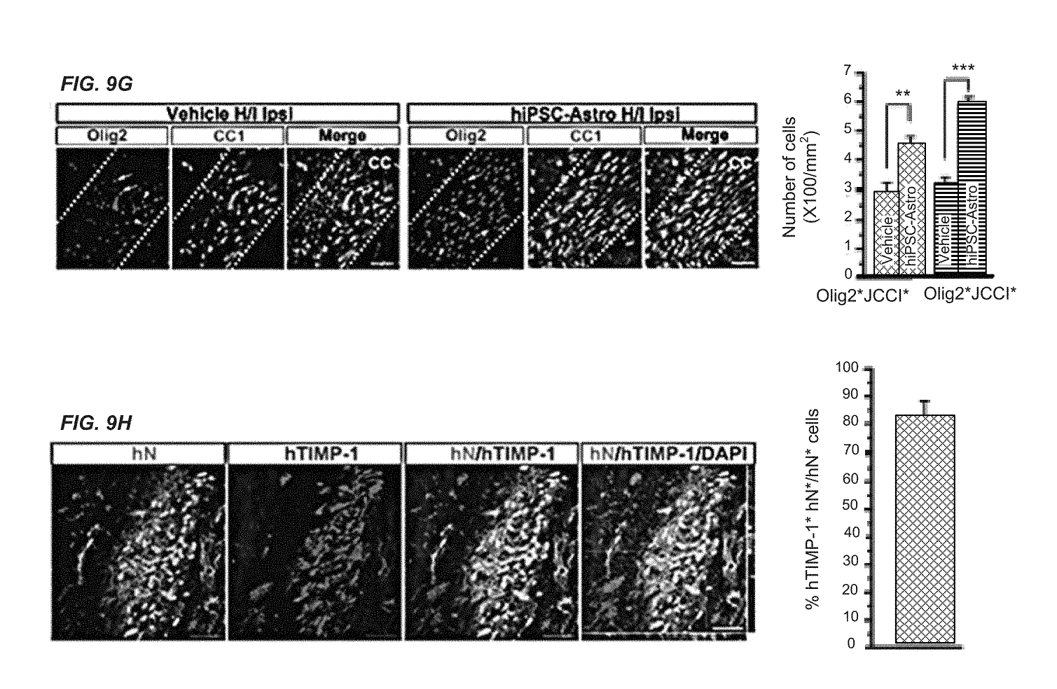

Considering the effects of immature hiPSC-Astros on OPCs in vitro, it was hypothesized that transplantation of immature hiPSC-Astros might promote myelination after PVL injury. To facilitate survival of transplanted cells, insults were induced in P6 Rag1−/− immunodeficient mice using unilateral carotid ligation followed with hypoxia, which resulted in selective injury to the subcortical white matter, without detectable injury to cortical neurons (Liu et al., 2011a; Shen et al., 2010). Consistent with previous studies (Liu et al., 2011a; Shen et al., 2010), reactive astrogliosis was observed at 4 days after induction of PVL injury selectively in the ipsilateral side of the brain (FIGS. 8A-8C). The contralateral side of the brain was not significantly changed after PVL injury and was used as a control. Due to the extremely low proliferation rate of mature hiPSC-Astros and hBrain-Astros (FIG. 2K) and low engraftment efficiency commonly seen from transplantation of terminally differentiated mature cells, it was technically difficult to collect ample mature human astrocytes and perform transplantation experiments with a good cell survival rate. Thus, immature hiPSC-Astros were only transplanted into the mouse brain at P7 when reactive astrocytes (FIG. 9A) started to be observed. Immature hiPSC-Astros were grafted to the periventricular area adjacent to the corpus callosum (CC) where hypo-myelination was observed (Liu et al., 2011a; Shen et al., 2010) (FIG. 9B). No tumor formation or overgrowth of transplanted cells was observed throughout the experiments. Transplanted hiPSC-Astros identified by human nuclei (hN) staining survived in the mouse brains at 4 days after transplantation (P11) (FIG. 9C). The majority of the transplanted cells were found located close to the hippocampus and the lateral ventricle. Similar to previous transplantation studies (Jiang et al., 2013b), a small percentage of the hiPSC-Astros expressed GFAP in vivo at 4 days after transplantation, as indicated by the double-labeling of GFAP and hN (FIG. 9C, 9.2±1.4% of the hN⁺ cells were GFAP⁺, n=4). The transplanted cells did not differentiate to oligodendrocytes, as indicated by expressing hCD44 but lack of MBP and Olig2 (FIGS. 9C and 8D). Next, the number of oligodendroglial lineage cells in the CC was examined. Consistent with previous studies (Jablonska et al., 2012; Reid et al., 2012; Segovia et al., 2008), in the vehicle control group, the number of Olig2⁺ oligodendroglia were increased in the ipsilateral side, compared to the contralateral side (FIGS. 9D and 9E). Transplantation of hiPSC-Astros further increased the number of Olig2⁺ cells in the CC of the ipsilateral side, but did not significantly affect the contralateral side (FIGS. 9D and 9E). Similar to previous studies (Liu et al., 2011a), severe hypo-myelination was observed selectively in the ipsilateral side at 4 days after PVL insult, as indicated by MBP staining (FIGS. 9D and 9F). Quantification of fluorescence intensity of MBP staining revealed that there was higher MBP immuno-positivity in ipsilateral side in the hiPSC-Astro group than those in the vehicle control group (FIGS. 9D and 9F). The number of immature and mature oligodendroglia was further examined, respectively identified by Olig2⁺/CC1⁻ and Olig2⁺/CC1⁺, in the ipsilateral sides of the vehicle and hiPSC-Astro groups. There were significantly more immature and mature oligodendroglia cells in the hiPSC-Astro group than in the vehicle group (FIG. 9G). To further confirm that transplanted immature hiPSC-Astros promoted myelination, the node of Ranvier and the density of myelinated axons in the ipsilateral side brains of the vehicle and hiPSC-Astro groups were examined. Consistently, there were more nodes of Ranvier, identified by βIV spectrin⁺ staining flanked by Caspr⁺ staining, in the hiPSC-Astro group, compared to the vehicle group (FIG. 8E). Moreover, the electron microscopic analyses demonstrated that the density of myelinated axons in the hiPSC-Astro group was significantly higher than that of the vehicle group (FIG. 8F). In addition, the majority of transplanted hiPSC-Astros abundantly expressed TIMP-1 at this time point (FIG. 9H). Immature TIMP-1^{siRNA} hiPSC-Astros were also transplanted, in which TIMP-1 expression was inhibited. MBP immuno-positivity in the ipsilateral sides at P11 in the TIMP-1^{siRNA} hiPSC-Astro and vehicle control groups was not significantly different (FIG. 8G), indicating that TIMP-1 secreted by transplanted hiPSC-Astros crucially contributed to the maturation of endogenous OPCs.

Transplanted Immature hiPSC-Astros Promoted Recovery of Behavioral Performance and Myelination

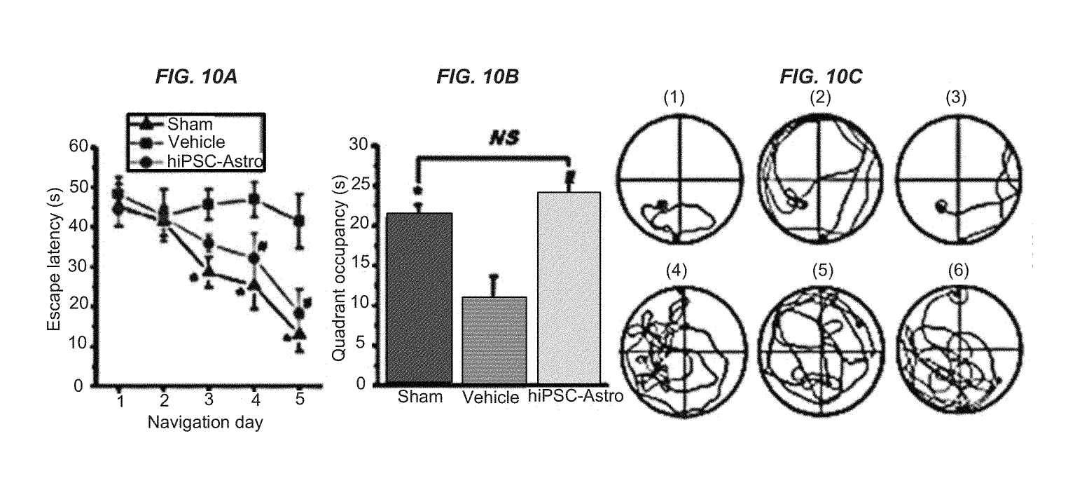

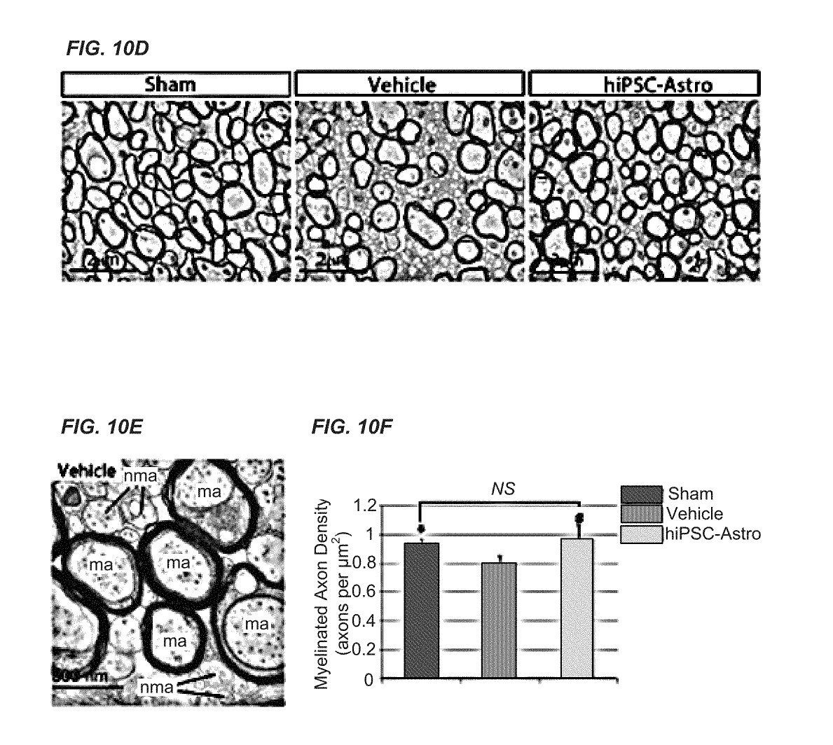

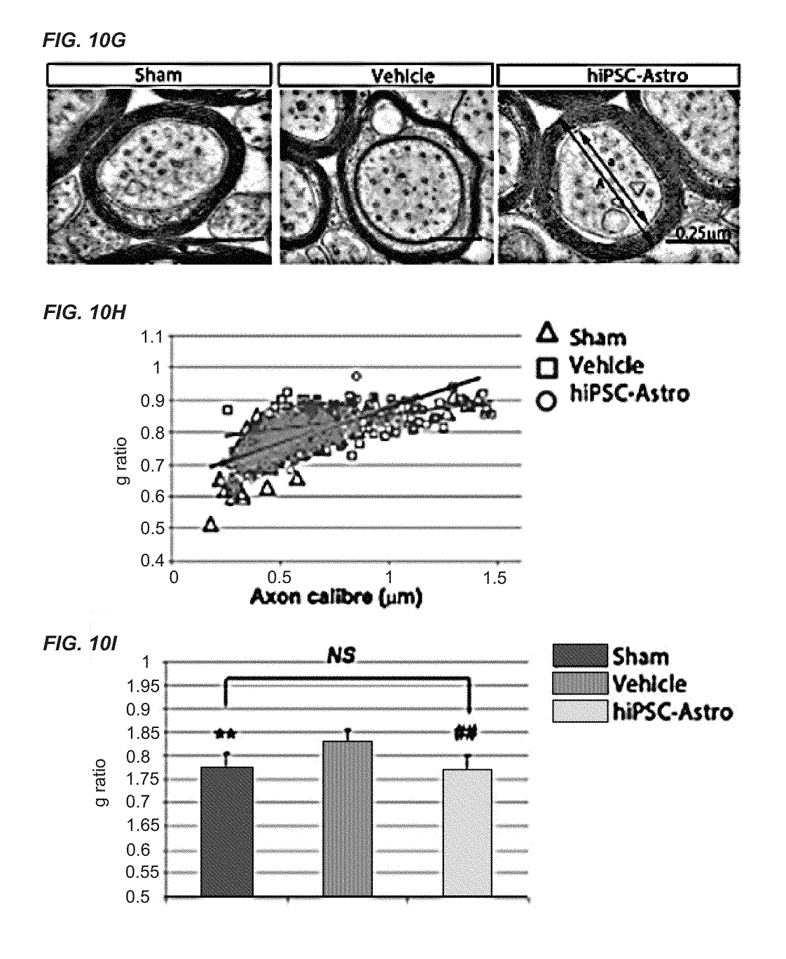

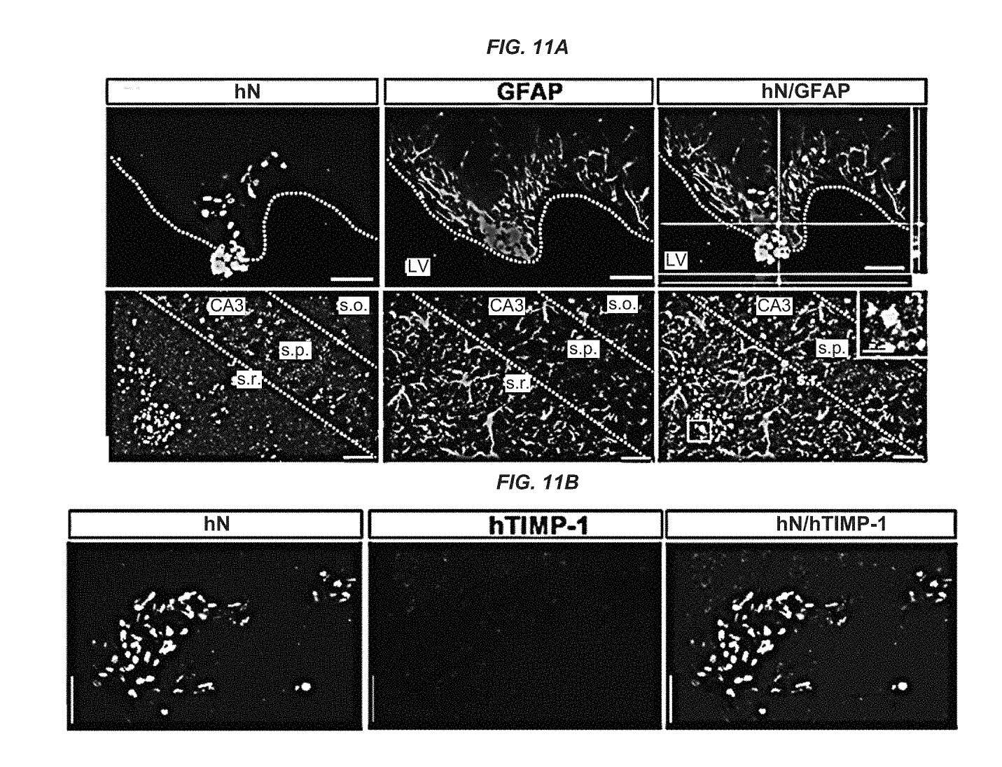

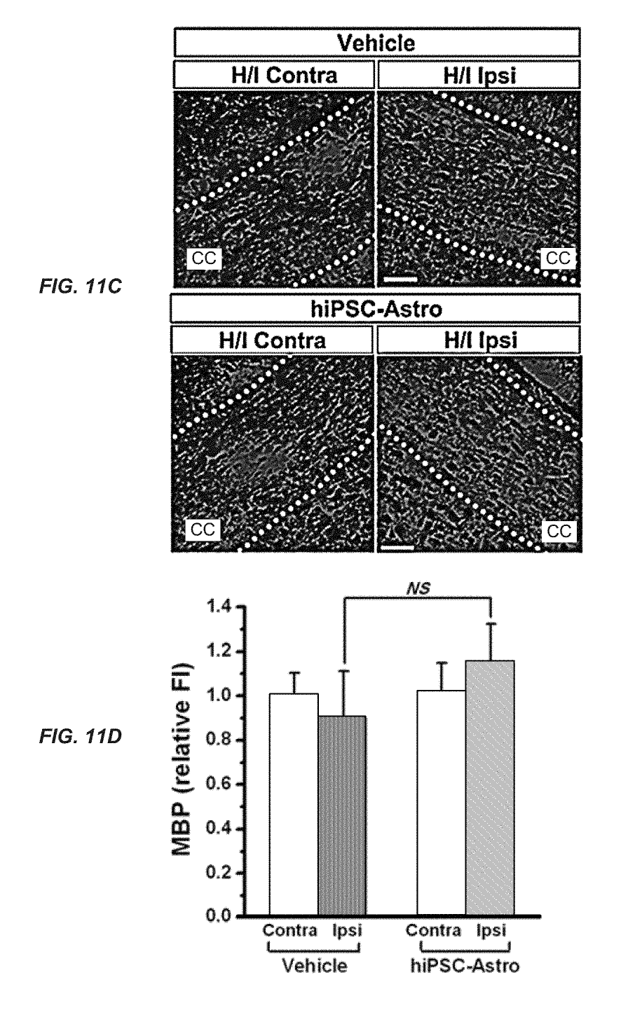

To examine the behavioral performance of the animals, three groups of animals were used: a sham group in which animals received sham PVL surgery and no vehicle PBS injection or cell transplantation, a vehicle group in which animals received PVL surgery and PBS injection after the surgery, and an hiPSC-Astro group in which animals received PVL surgery and hiPSC-Astro transplantation after the surgery. Consistent with a previous study, no significant difference was observed in motor functions in climbing on a wall with a slope angle of 45° (Liu et al., 2011a) from P11 to P21 among the three groups of animals. Myelin mass changes are highly associated with the higher functions of the brain (Liu et al., 2012). Accumulative studies have shown that myelination deficiency contributes to the development of long-term deficits in learning and memory in both mouse and rat models of PVL (Cengiz et al., 2011; Huang et al., 2009). Thus, learning and memory ability of P60 adult mice were examined in a Morris water maze. All mice were able to swim normally and locate the hidden platform during training trials in the Morris water maze test. As expected, mice in the vehicle group had worse performance and required more time to find the platform than those in the sham group. Analysis of escape latency revealed significant differences among the sham, vehicle, and hiPSC-Astro groups (FIG. 10A and items (1)-(3) of FIG. 10C). On navigation days 3 to 5, mice in the sham group showed less escape latency compared to mice in the vehicle group. Notably, on navigation day 4 and 5, mice in the hiPSC-Astro group showed better performance compared to mice in the vehicle group. In the probe trials, mice in the sham and hiPSC-Astro groups showed better performance and spent significantly more time than the vehicle group in the quadrant where the platform had been (FIG. 10B and items (4)-(6) of FIG. 10C). Moreover, the distribution of transplanted hiPSC-Astros in the P60 animals was examined. hN⁺ cells were found close to the lateral ventricle and integrated into the hippocampus, close to the CA3 region (FIG. 11A). These transplanted cells maintained their astroglial lineage properties, as indicated by over half of them expressing GFAP (58.7±3.4%, n=7). However, at this time point, expression of TIMP-1 was undetectable in these transplanted hiPSC-Astros (FIG. 11B). Abnormal cellular hypertrophy and massive proliferation of the vast majority of transplanted hiPSC-Astros were not observed, indicating that the transplanted hiPSC-Astro did not become reactive astrocytes.

Next, to further explore the differences in myelination that might contribute to the changes in behavioral performance, MBP expression was examined in the vehicle and hiPSC-Astro groups at P60. No significant difference was found in MBP immuno-positivity between the contralateral and ipsilateral sides from both the vehicle and hiPSC-Astro groups (FIGS. 11C and 11D). Previous studies have demonstrated that cellular recovery in oligodendrocytes does not correlate with proper axonal myelination (Jablonska et al., 2012). Thus, electron microscopy was used to determine whether in this model, neonatal hypoxic-ischemic injury caused abnormalities in ultrastructure of myelinated axons at P60. It was found that unmyelinated axons were distributed among myelinated axons in the vehicle group (FIGS. 10D and 10E). Compared to the sham group, the density of myelinated axons in the vehicle group was significantly lower (FIG. 10F). The density of myelinated axons in the hiPSC-Astro group was similar to that of the sham group and was significantly higher than that of the vehicle group (FIG. 10F). Under higher magnification, some myelinated axons exhibited thinner myelin sheath in the vehicle group, compared to the sham and hiPSC-Astro groups (FIG. 10G). The scatter plot graph of g ratio analysis in FIG. 10H demonstrates that the g ratio values from the vehicle group were mostly between 0.8-0.9; however, the g ratio values from the sham and hiPSC-Astro groups were largely overlapped and were mostly between 0.7-0.8, with some being around 0.6. Notably, the majority of low caliber axons were unmyelinated in the vehicle group (FIG. 10D) and thus many myelinated axons with large diameters were included for g ratio analysis (FIG. 10H). Compared to the vehicle group, the sham and hiPSC-Astro groups had significantly lower g ratio values (FIG. 10I). The effects of transplanted hiPSC-Astros on both the density of myelinated axons and g ratio indicates their impact on axons, in addition to their impact on oligodendroglia myelination. Moreover, the significant but small differences in the density of myelinated axons among the groups suggests that in all conditions, oligodendrocytes were able to mature into myelin-forming cells but the maturation was accelerated in the sham and hiPSC-Astro groups, compared with the vehicle group.

Intranasal Administration of Immature hiPSC-Astro ACM Promoted OPC Maturation after PVL Injury

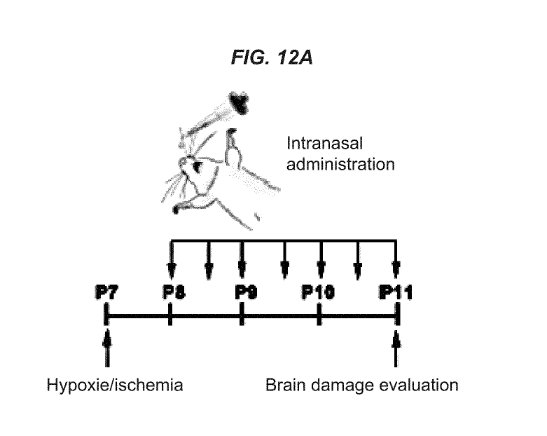

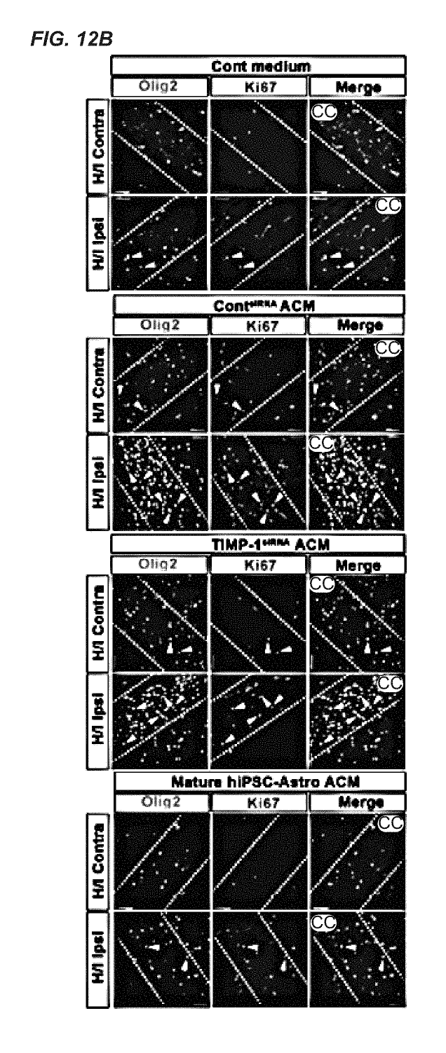

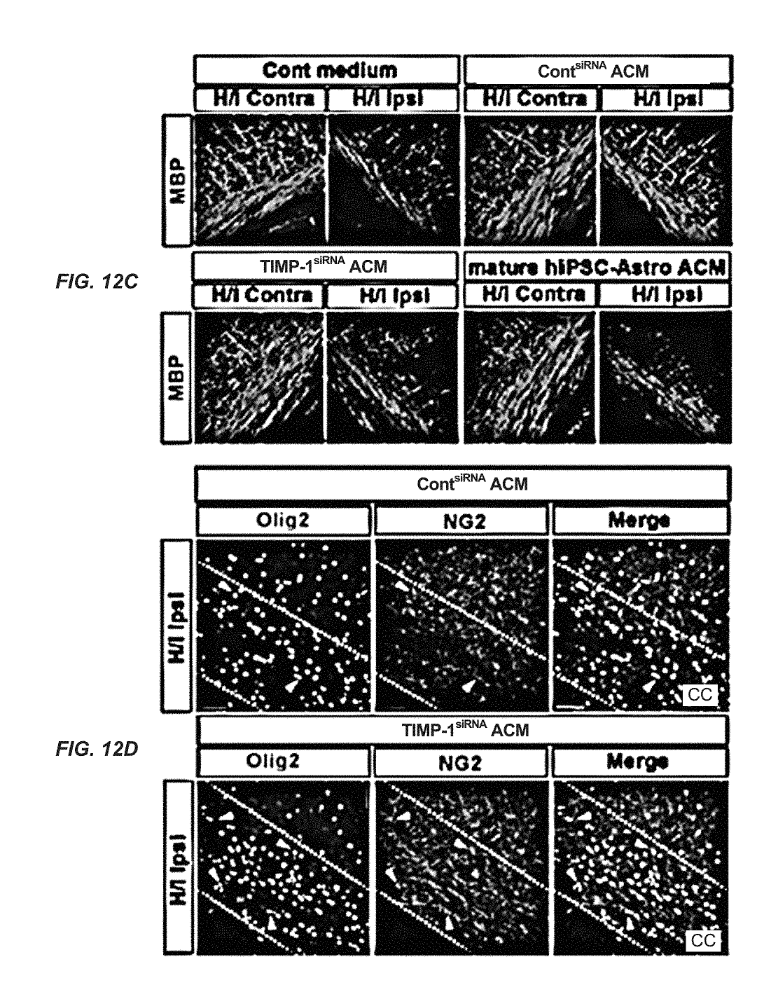

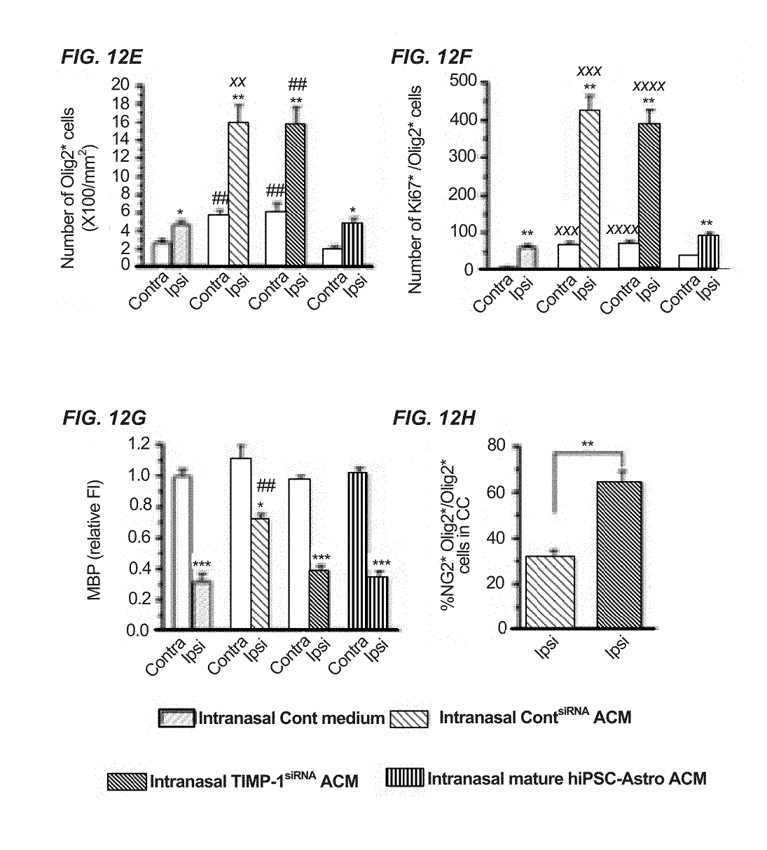

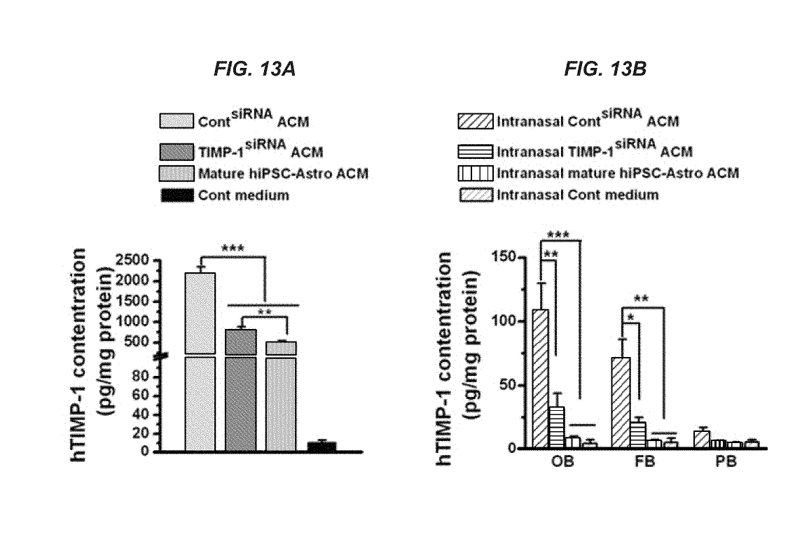

To further compare the effects of immature and mature human astrocytes on OPC maturation after PVL injury and to investigate the role of TIMP-1, the total concentrated factors collected from ACM was intranasally administered. Similar to the mouse PVL model, unilateral carotid ligation followed by hypoxia (i.e., 6% O₂ for 1 hour) in rats at P7 also resulted in selective white matter injury without evident injury to cortical neurons (Follett et al., 2000). Here the rat model of PVL was used to facilitate intranasal administration because neonatal rats have more easily visible nares than neonatal mice. Starting from P8, control (Cont) medium that had not been exposed to cells, concentrated mature hiPSC-Astro ACM, as well as Cont^{siRNA} ACM and TIMP-1^{siRNA} ACM, collected from immature hiPSC-Astros transfected with control and TIMP-1 siRNA, respectively (FIG. 12A) were applied. The ELISA assay confirmed the higher abundance of human TIMP-1 in Cont^{siRNA} ACM than that in TIMP-1^{siRNA} ACM or mature hiPSC-Astro ACM (FIG. 13A). The intranasal route allowed effective delivery directly to the brain from the nasal mucosa, as indicated by ELISA assay of the brain tissues collected at 1 hour after the last intranasal administration (FIG. 13B). At P11, it was found that administration of both Cont^{siRNA} and TIMP-1^{siRNA} ACM significantly increased the number of Olig2⁺ oligodendroglia not only in the ipsilateral side, but also in the contralateral side of the brain, compared to administration of Cont medium and mature hiPSC-Astro ACM (FIGS. 12B and 12E). There were also more proliferating oligodendroglial cells, identified by Ki67⁺ and Olig2⁺, in both the ipsilateral and contralateral sides in Cont^{siRNA} and TIMP-1^{siRNA} ACM groups than those in the Cont medium and mature iPSC-Astro ACM groups (FIGS. 12B and 12F). MBP staining revealed that there was higher MBP immuno-positivity in the ipsilateral side brain from the Cont^{siRNA} ACM group than in the Cont medium group (FIGS. 12C and 12G), indicating that ACM from immature hiPSC-Astro promoted OPC maturation. Administration of TIMP-1^{siRNA} ACM or mature hiPSC-Astro ACM was not able to promote OPC maturation after PVL injury (FIGS. 12C and 12G). Since there was an increased number of Olig2⁺ cells but not MBP immuno-positivity in the ipsilateral side from the TIMP-1^{siRNA} ACM group, it was further investigated whether these cells stayed at a progenitor stage. In the CC of the ipsilateral side, more Olig2⁺ cells expressing OPC marker NG2 were found in the TIMP-1^{siRNA} ACM group than in the Cont^{siRNA} ACM group (FIGS. 12D and 12H). Together, these results demonstrate that released factors such as TIMP-1, mediated the effects of immature hiPSC-Astros on promoting OPC maturation after PVL injury.

### Discussion

By differentiating hPSCs to astrocytes with defined immature and mature phenotypes, described herein is a novel regulation of oligodendrocyte development by astrocytes at a previously understudied immature stage. This work demonstrated that hiPSC-derived immature astroglial transplant or ACM promoted myelinogenesis and improved behavioral outcome in animal models of PVL. These results implicate a novel strategy for promoting myelinogenesis by hiPSC-derived immature astroglia.

Due to a lack of efficient methods for purifying and maintaining astrocytes with defined immature and mature phenotypes in culture (Foo et al., 2011), no specific studies have been performed to examine how astrocytes at specific developmental stages interact with oligodendrocytes. Krencik et al. (2011) showed that hPSC-Astros become mature in a serum-free condition after a long period of time (180 days). Roybon et al. (2013) reported that hPSC-Astros cultured in a serum-containing medium (1% fetal bovine serum) exhibit mature phenotypes after a short exposure to FGF1 (7 days). Here, maturation of hiPSC-Astros was promoted in a serum-free medium containing FGF1. It was found that after a 30- to 50-day culture, immature hiPSC-Astros became mature, as indicated by increased EAAT1 expression and glutamate uptake, and decreased expression of NF1A, hCD44 and vimentin, consistent with the observations of astrocyte maturation in human tissue (Bjorklund et al., 1984; Girgrah et al., 1991; Yamada et al., 1992). Moreover, immature and mature hiPSC-Astros also possessed forebrain identity similar to hBrain-Astros. Thus, using the protocol described herein, immature and mature human astrocytes could be efficiently derived from hPSCs, providing new opportunities to study human astrocyte development and developmental interactions between astroglia and oligodendroglia.

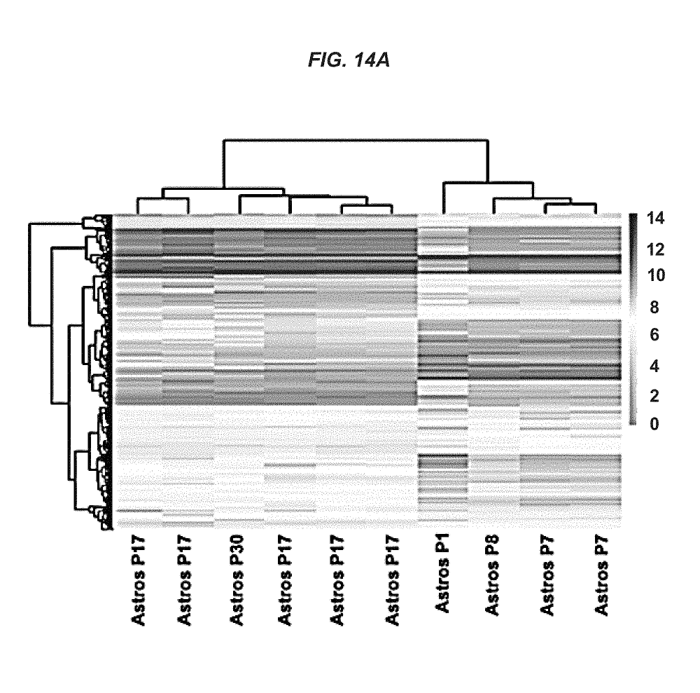

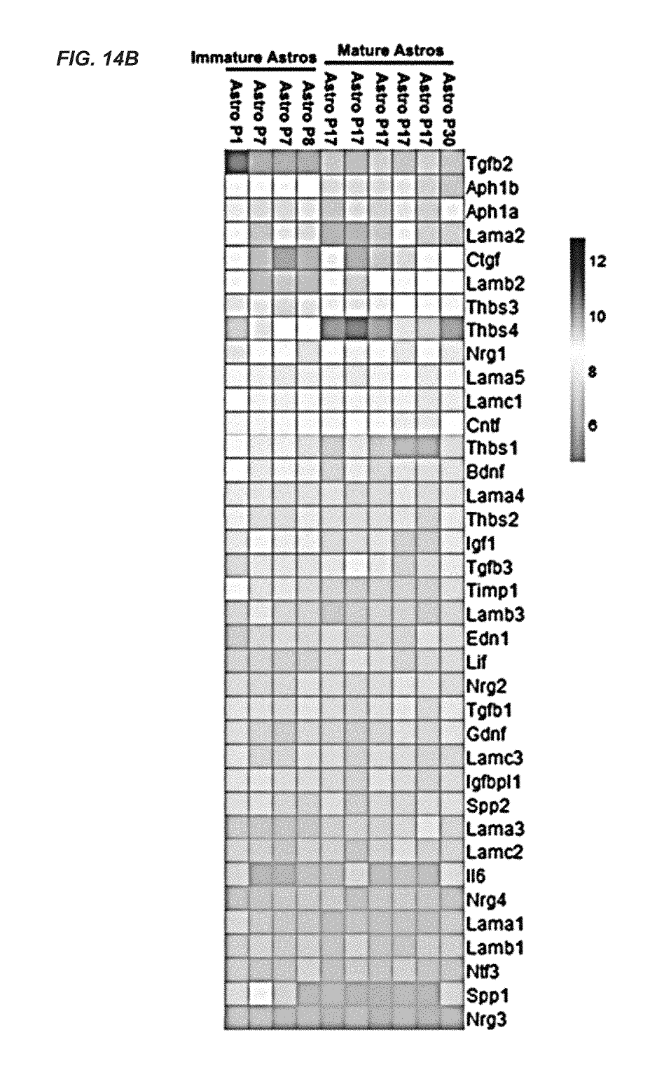

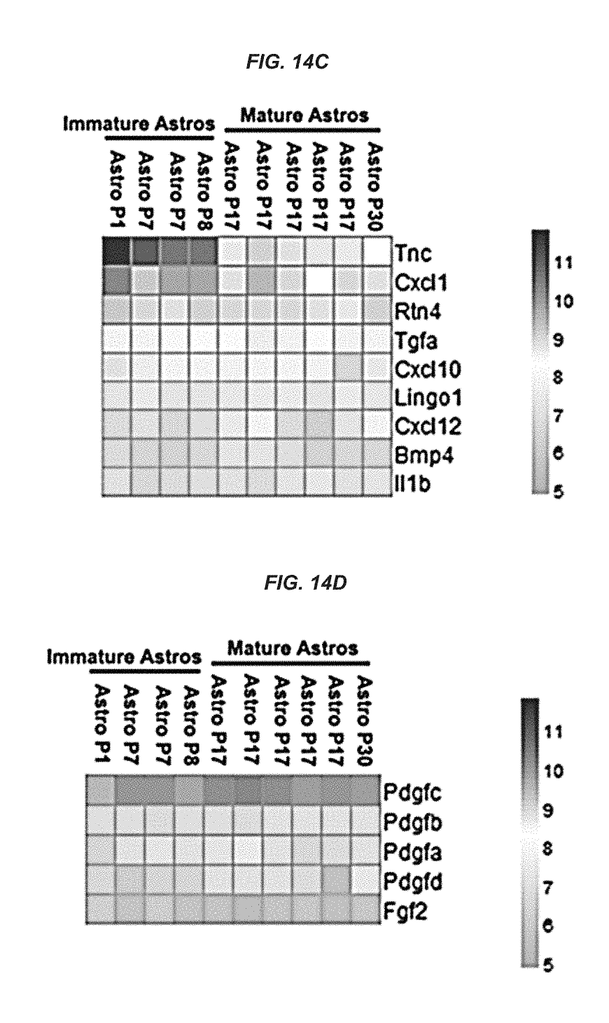

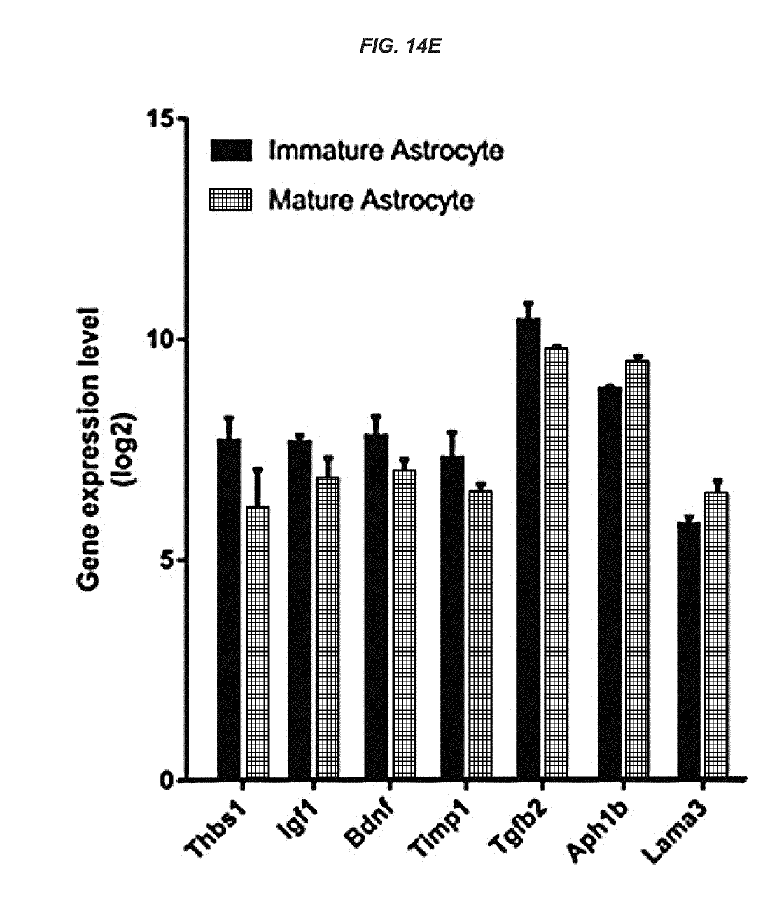

The microarray gene analyses showed that immature human astrocytes expressed gene transcripts encoding OPC mitogens and factors that inhibit OPC maturation to myelinating oligodendrocytes. The data presented herein indicate that the inhibitory factors and OPC mitogens worked synergistically to promote OPC proliferation. Moreover, immature astrocytes expressed gene transcripts encoding factors that promote OPC maturation and myelination. By a combination of qPCR, siRNA knockdown, and intranasal ACM administration experiments, both in vitro and in vivo evidence was provided that TIMP-1 partially but critically mediates the effects of immature astrocytes on OPC differentiation, but not the effects on OPC proliferation. Hence, the effects of hiPSC-Astros on oligodendroglia could depend on which stage the oligodendroglia cells are at and which astrocyte-secreted factors the oligodendroglia cells are exposed to. The gene expression of secreted factors with multifaceted effects on OPCs indicates fine regulatory effects of the immature astrocytes on oligodendroglial lineage progression. In addition, by analyzing a transcriptome database of mouse astrocytes (Cahoy et al., 2008), it was consistently found that mouse immature astrocytes also expressed gene transcripts encoding factors that promoted or inhibited myelination, and promoted OPC proliferation (FIGS. 14A-14D). Interestingly, Tgfb2, encoding transforming growth factor β2 (TGFβ2), but not Timp-1, was prominently expressed in immature mouse astrocytes (FIGS. 14B and 14E). Previous studies have demonstrated that the TGFβ signaling pathway critically promotes oligodendrocyte differentiation (Palazuelos et al., 2014).

The in vitro findings described herein indicated that immature hiPSC-Astros not only promoted the OPC proliferation but also robustly boosted OPC differentiation to oligodendrocytes. It was further demonstrated that forebrain immature hiPSC-derived astroglial transplants rescued the hypo-myelination in a hypoxic/ischemic mouse model of PVL. TIMP-1 expression was found to be abundant in transplanted hiPSC-Astros at 4 days after transplantation and was undetectable at about 2 months after transplantation, which is consistent with an in vitro observation (FIG. 7D) and previously reported developmental regulation of TIMP-1 expression in astrocytes (Ulrich et al., 2005). Furthermore, the study of intranasal administration of Cont^{siRNA} and TIMP-1^{siRNA} ACM pinpointed the role of TIMP-1 in mediating the effects of ACM on promoting OPC differentiation in vivo. Taken together, through released factors such as TIMP-1, hiPSC-Astros promoted myelination and behavioral recovery.

Intracerebral cell transplantation during the neonatal period is not ideal in clinical settings. Here, it was demonstrated that direct application of hiPSC-Astro ACM via intranasal administration promoted myelination after PVL injury. This example thus demonstrates an hiPSC-based cell-free therapy. This approach is particularly useful in myelin disorders such as multiple sclerosis, where an inflamed environment significantly compromises survival of transplanted cells. Administration of the cell-free, concentered factors that are released from human immature astrocytes is effective in promoting remyelination (Chen et al., 2014b).

### Experimental Procedures

Culture of hiPSC

The two hiPSC lines, hiPSC1 and hiPSC2, were reprogrammed from healthy individuals' fibroblasts by using retroviruses encoding OCT4, SOX2, KLF4 and c-MYC (FIG. 1A) (Chen et al., 2014a). All experiments conducted on hPSCs adhered to approved Stem Cell Research Oversight Committee at the University of California, Davis.

### Differentiation and Culture of Human Astrocytes

Embryoid body-based differentiation procedure was used for astroglial differentiation of hiPSCs (FIG. 1B). The hBrain-Astros were isolated from the cerebral cortex of fetal human brain (ScienCell; Catalog number: 1800).

### Generation of Astrocyte-Conditioned Medium

ACM was concentrated 50-fold using centrifugal concentrators (Millipore). Protein concentration was determined by BCA assay (Thermo Scientific) and ACM was fed to primary mixed neuron/glia culture at 100 μg/mL. TIMP-1^{siRNA} ACM supplemented with TIMP-1 (10 ng/mL; Peprotech) was also fed to the primary culture.

### Immunostaining

Cells were fixed with 4% paraformaldehyde and brain sections (i.e., 18 μm thick) from P11-P60 mice and rats were processed for immunofluorescence staining (Liu et al., 2011b). The information for primary antibodies and dilutions are listed in Table 2. Images were captured using a Nikon Eclipse C1 or Nikon A1 confocal laser-scanning microscope.

### Microarray Analysis and Heat Maps

Illumina bead array was performed for gene expression analysis (Liu et al., 2006). Array data were processed using Illumina GenomeStudio software (Illumina).

### PVL Animal Model

Animal experiments were performed following protocols approved by the Animal Care and Use Committees at the University of California, Davis. By unilateral carotid ligation (UCL) followed by hypoxia, hypoxic/ischemic insults were induced in P6 mouse pups of Rag1−/− immunodeficient mice (B6.12957-Rag1tm1Mom on a C57BL/6 background, Jackson Lab) or P7 Long-Evans rat pups (Charles Rivers laboratories) (Follett et al., 2000).

### Cell Transplantation

One day after induction of hypoxic/ischemic injury in mice (P7), cell transplantation was performed (FIG. 9A). A Hamilton syringe and needle were used to deliver cells by inserting through the skull and into the target site (Chen et al., 2014a).

### Intranasal Administration

One day after induction of hypoxic/ischemic injury in rats, intranasal administration of concentrated ACM was performed (FIG. 12A). Control medium or ACM was administered every 12 hours from P8 to P11.

### Electron Microscopy

Brain tissues were fixed and sectioned as previously described (Jiang et al., 2013a). Electron microscopy (EM) images were captured using a high-resolution CCD camera (Gatan, Pleasanton, Calif.). Images were processed using DigitalMicrograph (Gatan). EM images were analyzed using ImageJ software.

### Morris Water-Maze Test

The Morris water-maze test was performed with mice at P60 (Jiang et al., 2013b).

### Data Analysis

For all experiments, analysis was derived from at least three independent experiments. All data are represented as mean ±S.E.M. The escape latency in the behavioral training tests was determined by two-way repeated measures analysis of variance (ANOVA). All other assessments were analyzed using the Student's t test when only two groups were compared, or one-way ANOVA when three or more groups were compared.

### References

Back, S. A., Tuohy, T. M., Chen, H., Wallingford, N., Craig, A., Struve, J., Luo, N. L., Banine, F., Liu, Y., Chang, A., et al. (2005). Hyaluronan accumulates in demyelinated lesions and inhibits oligodendrocyte progenitor maturation. Nat Med 11, 966-972.

Barres, B. A. (2008). The mystery and magic of glia: a perspective on their roles in health and disease. Neuron 60, 430-440.

Bjorklund, H., Eriksdotter-Nilsson, M., Dahl, D., and Olson, L. (1984). Astrocytes in smears of CNS tissues as visualized by GFA and vimentin immunofluorescence. Med Biol 62, 38-48.

Cahoy, J. D., Emery, B., Kaushal, A., Foo, L. C., Zamanian, J. L., Christopherson, K. S., Xing, Y., Lubischer, J. L., Krieg, P. A., Krupenko, S. A., et al. (2008). A transcriptome database for astrocytes, neurons, and oligodendrocytes: a new resource for understanding brain development and function. J Neurosci 28, 264-278.

Cengiz, P., Uluc, K., Kendigelen, P., Akture, E., Hutchinson, E., Song, C., Zhang, L., Lee, J., Budoff, G. E., Meyerand, E., et al. (2011). Chronic neurological deficits in mice after perinatal hypoxia and ischemia correlate with hemispheric tissue loss and white matter injury detected by MRI. Dev Neurosci 33, 270-279.

Chen, C., Chan, A., Wen, H., Chung, S. H., Deng, W., and Jiang, P. (2015). Stem and Progenitor Cell-Derived Astroglia Therapies for Neurological Diseases. Trends Mol Med 21, 715-729.

Chen, C., Jiang, P., Xue, H., Peterson, S. E., Tran, H. T., McCann, A. E., Parast, M. M., Li, S., Pleasure, D. E., Laurent, L. C., et al. (2014a). Role of astroglia in Down's syndrome revealed by patient-derived human-induced pluripotent stem cells. Nat Commun 5, 4430.

Chen, L., Coleman, R., Leang, R., Tran, H., Kopf, A., Walsh, C. M., Sears-Kraxberger, I., Steward, O., Macklin, W. B., Loring, J. F., et al. (2014b). Human neural precursor cells promote neurologic recovery in a viral model of multiple sclerosis. Stem Cell Rep 2, 825-837.

Chung, W. S., Clarke, L. E., Wang, G. X., Stafford, B. K., Sher, A., Chakraborty, C., Joung, J., Foo, L. C., Thompson, A., Chen, C., et al. (2013). Astrocytes mediate synapse elimination through MEGF10 and MERTK pathways. Nature 504, 394-400.

Dahl, D., Rueger, D. C., Bignami, A., Weber, K., and Osborn, M. (1981). Vimentin, the 57 000 molecular weight protein of fibroblast filaments, is the major cytoskeletal component in immature glia. Eur J Cell Biol 24, 191-196.

Deneen, B., Ho, R., Lukaszewicz, A., Hochstim, C. J., Gronostajski, R. M., and Anderson, D. J. (2006). The transcription factor NFIA controls the onset of gliogenesis in the developing spinal cord. Neuron 52, 953-968.

Deng, W. (2010). Neurobiology of injury to the developing brain. Nat Rev Neurol 6, 328-336.

Emdad, L., D'Souza, S. L., Kothari, H. P., Qadeer, Z. A., and Germano, I. M. (2011). Efficient differentiation of human embryonic and induced pluripotent stem cells into functional astrocytes. Stem Cells Dev 21, 404-410.

Fancy, S. P., Harrington, E. P., Yuen, T. J., Silbereis, J. C., Zhao, C., Baranzini, S. E., Bruce, C. C., Otero, J. J., Huang, E. J., Nusse, R., et al. (2011). Axin2 as regulatory and therapeutic target in newborn brain injury and remyelination. Nat Neurosci 14, 1009-1016.

Follett, P. L., Rosenberg, P. A., Volpe, J. J., and Jensen, F. E. (2000). NBQX attenuates excitotoxic injury in developing white matter. J Neurosci 20, 9235-9241.

Foo, L. C., Allen, N. J., Bushong, E. A., Ventura, P. B., Chung, W. S., Zhou, L., Cahoy, J. D., Daneman, R., Zong, H., Ellisman, M. H., et al. (2011). Development of a method for the purification and culture of rodent astrocytes. Neuron 71, 799-811.

Franklin, R. J., Crang, A. J., and Blakemore, W. F. (1991). Transplanted type-1 astrocytes facilitate repair of demyelinating lesions by host oligodendrocytes in adult rat spinal cord. J Neurocytol 20, 420-430.

Gallo, V., and Deneen, B. (2014). Glial development: the crossroads of regeneration and repair in the CNS. Neuron 83, 283-308.

Girgrah, N., Letarte, M., Becker, L. E., Cruz, T. F., Theriault, E., and Moscarello, M. A. (1991). Localization of the CD44 glycoprotein to fibrous astrocytes in normal white matter and to reactive astrocytes in active lesions in multiple sclerosis. J Neuropathol Exp Neurol 50, 779-792.

Haynes, R. L., Folkerth, R. D., Keefe, R. J., Sung, I., Swzeda, L. I., Rosenberg, P. A., Volpe, J. J., and Kinney, H. C. (2003). Nitrosative and oxidative injury to premyelinating oligodendrocytes in periventricular leukomalacia. J Neuropathol Exp Neurol 62, 441-450.

Huang, Z., Liu, J., Cheung, P. Y., and Chen, C. (2009). Long-term cognitive impairment and myelination deficiency in a rat model of perinatal hypoxic-ischemic brain injury. Brain Res 1301, 100-109.

Jablonska, B., Scafidi, J., Aguirre, A., Vaccarino, F., Nguyen, V., Borok, E., Horvath, T. L., Rowitch, D. H., and Gallo, V. (2012). Oligodendrocyte regeneration after neonatal hypoxia requires FoxO1-mediated p27Kip1 expression. J Neurosci 32, 14775-14793.

Jiang, P., Chen, C., Liu, X. B., Selvaraj, V., Liu, W., Feldman, D. H., Liu, Y., Pleasure, D. E., Li, R. A., and Deng, W. (2013a). Generation and characterization of spiking and nonspiking oligodendroglial progenitor cells from embryonic stem cells. Stem Cells 31, 2620-2631.

Jiang, P., Chen, C., Wang, R., Chechneva, O. V., Chung, S. H., Rao, M. S., Pleasure, D. E., Liu, Y., Zhang, Q., and Deng, W. (2013b). hESC-derived Olig2+ progenitors generate a subtype of astroglia with protective effects against ischaemic brain injury. Nat Commun 4, 2196.

Krencik, R., Weick, J. P., Liu, Y., Zhang, Z. J., and Zhang, S. C. (2011). Specification of transplantable astroglial subtypes from human pluripotent stem cells. Nat Biotechnol 29, 528-534.

Liu, J., Dietz, K., DeLoyht, J. M., Pedre, X., Kelkar, D., Kaur, J., Vialou, V., Lobo, M. K., Dietz, D. M., Nestler, E. J., et al. (2012). Impaired adult myelination in the prefrontal cortex of socially isolated mice. Nature Neurosci 15, 1621-1623.

Liu, W., Shen, Y., Plane, J. M., Pleasure, D. E., and Deng, W. (2011a). Neuroprotective potential of erythropoietin and its derivative carbamylated erythropoietin in periventricular leukomalacia. Exp Neurol 230, 227-239.

Liu, Y., Han, S. S., Wu, Y., Tuohy, T. M., Xue, H., Cai, J., Back, S. A., Sherman, L. S., Fischer, I., and Rao, M. S. (2004). CD44 expression identifies astrocyte-restricted precursor cells. Dev Biol 276, 31-46.

Liu, Y., Jiang, P., and Deng, W. (2011b). OLIG gene targeting in human pluripotent stem cells for motor neuron and oligodendrocyte differentiation. Nat Prot 6, 640-655.

Liu, Y., Shin, S., Zeng, X., Zhan, M., Gonzalez, R., Mueller, F. J., Schwartz, C. M., Xue, H., Li, H., Baker, S. C., et al. (2006). Genome wide profiling of human embryonic stem cells (hESCs), their derivatives and embryonal carcinoma cells to develop base profiles of U.S. Federal government approved hESC lines. BMC Dev Biol 6, 20.

Molofsky, A. V., Krencik, R., Ullian, E. M., Tsai, H. H., Deneen, B., Richardson, W. D., Barres, B. A., and Rowitch, D. H. (2012). Astrocytes and disease: a neurodevelopmental perspective. Genes Dev 26, 891-907.

Moore, C. S., Milner, R., Nishiyama, A., Frausto, R. F., Serwanski, D. R., Pagarigan, R. R., Whitton, J. L., Miller, R. H., and Crocker, S. J. (2011). Astrocytic tissue inhibitor of metalloproteinase-1 (TIMP-1) promotes oligodendrocyte differentiation and enhances CNS myelination. J Neurosci 31, 6247-6254.

Nash, B., Thomson, C. E., Linington, C., Arthur, A. T., McClure, J. D., McBride, M. W., and Barnett, S. C. (2011). Functional duality of astrocytes in myelination. J Neurosci 31, 13028-13038.

Noble, M., Davies, J. E., Mayer-Proschel, M., Proschel, C., and Davies, S. J. (2011). Precursor cell biology and the development of astrocyte transplantation therapies: lessons from spinal cord injury. Neurotherapeutics 8, 677-693.

Palazuelos, J., Klingener, M., and Aguirre, A. (2014). TGFbeta signaling regulates the timing of CNS myelination by modulating oligodendrocyte progenitor cell cycle exit through SMAD3/4/FoxO1/Spl. J Neurosci 34, 7917-7930.

Patterson, M., Chan, D. N., Ha, I., Case, D., Cui, Y., Van Handel, B., Mikkola, H. K., and Lowry, W. E. (2012). Defining the nature of human pluripotent stem cell progeny. Cell Res 22, 178-193.

Pekny, M., and Pekna, M. (2014). Astrocyte Reactivity and Reactive Astrogliosis: Costs and Benefits. Physiol Rev 94, 1077-1098.

Reid, M. V., Murray, K. A., Marsh, E. D., Golden, J. A., Simmons, R. A., and Grinspan, J. B. (2012). Delayed myelination in an intrauterine growth retardation model is mediated by oxidative stress upregulating bone morphogenetic protein 4. J Neuropathol Exp Neurol 71, 640-653.

Roybon, L., Lamas, N. J., Garcia-Diaz, A., Yang, E. J., Sattler, R., Jackson-Lewis, V., Kim, Y. A., Kachel, C. A., Rothstein, J. D., Przedborski, S., et al. (2013). Human stem cell-derived spinal cord astrocytes with defined mature or reactive phenotypes. Cell Rep 4, 1035-1048.

Segovia, K. N., McClure, M., Moravec, M., Luo, N. L., Wan, Y., Gong, X., Riddle, A., Craig, A., Struve, J., Sherman, L. S., et al. (2008). Arrested oligodendrocyte lineage maturation in chronic perinatal white matter injury. Annals Neurol 63, 520-530.

Shaltouki, A., Peng, J., Liu, Q., Rao, M. S., and Zeng, X. (2013). Efficient Generation of Astrocytes From Human Pluripotent Stem Cells in Defined Conditions. Stem Cells 31, 941-952.

Shen, Y., Plane, J. M., and Deng, W. (2010). Mouse models of periventricular leukomalacia. J Vis Exp, 39, pii: 1951. http://www.jove.com/index/Details.stp?ID=1951, doi: 10.3791/1951. PubMed ID: 20485263.

Sim, F. J., McClain, C. R., Schanz, S. J., Protack, T. L., Windrem, M. S., and Goldman, S. A. (2011). CD140a identifies a population of highly myelinogenic, migration-competent and efficiently engrafting human oligodendrocyte progenitor cells. Nat Biotechnol 29, 934-941.

Talbott, J. F., Loy, D. N., Liu, Y., Qiu, M. S., Bunge, M. B., Rao, M. S., and Whittemore, S. R. (2005). Endogenous Nkx2.2+/Olig2+ oligodendrocyte precursor cells fail to remyelinate the demyelinated adult rat spinal cord in the absence of astrocytes. Exp Neurol 192, 11-24.

Ulrich, R., Gerhauser, I., Seeliger, F., Baumgartner, W., and Alldinger, S. (2005). Matrix metalloproteinases and their inhibitors in the developing mouse brain and spinal cord: a reverse transcription quantitative polymerase chain reaction study. Dev Neurosci 27, 408-418.

Yamada, T., Kawamata, T., Walker, D. G., and McGeer, P. L. (1992). Vimentin immunoreactivity in normal and pathological human brain tissue. Acta Neuropathologica 84, 157-162.

### Supplemental Experimental Procedures

Culture of hiPSCs

The hPSCs, including hiPSCs and OLIG2-GFP hESCs were maintained on irradiated mouse embryonic fibroblasts (Millipore) in DMEM/F12 medium with 20% knockout serum replacement, 0.1 mM β-mercaptoethanol, 1×nonessential amino acid, 1 mM L-glutamine (Gibco) and 20 ng/ml FGF2 (Peprotech) (Liu et al., 2011b). For feeder depletion, cells were passaged by collagenase (type IV, 1 mg/mL; Invitrogen) treatment, and then hiPSC colonies were plated on Matrigel (BD Biosciences)-coated plate with mTeSR1 media (STEMCELL Technologies).

Differentiation and culture of human astrocytes

The schematic diagram in FIG. 1B shows the procedure for astroglial differentiation of hiPSCs. Embryoid bodies (EBs) were grown in a suspension culture in DMEM/F12, supplemented with 1×N2 (Invitrogen) for 7 days. EBs were then plated on growth factor reduced Matrigel (BD Biosciences)-coated plates in the presence of neural induction medium consisting of DMEM/F12, 1×N2 and laminin (1 μg/mL; Sigma). NPCs in the form of rosettes developed for another 7 days (day 14). Next, rosettes were manually isolated from surrounding cells and expanded as neurospheres in a suspension culture for 6 days (day 20) in NPC medium, composed of DMEM/F12, 1×N2, 1×B27-RA (Invitrogen) and 20 ng/mL FGF2 (Peprotech). Then, the neurospheres were dissociated into single cells and were attached with a substrate of poly-L-ornithine (0.002%) and fibronectin (10 μg/mL; Millipore) in the chemically defined and xeno-free astroglial medium containing DMEM/F12, 1×N2, 1×B27-RA, BMP4 (10 ng/mL; Peprotech) and FGF2 (20 ng/mL) for directed astroglial differentiation (Chen et al., 2014; Jiang et al., 2013c). Medium was changed every other day. NPC-Astros and Olig2PC-Astros were differentiated from Olig2-GFP hESCs, as described in a previous study (Jiang et al., 2013c). Briefly, Olig2-GFP hESCs were differentiated to NPCs with the treatment of purmorphamine (1 μM; Cayman Chemical). The Olig2−/GFP− and Olig2+/GFP+ NPCs were purified by using fluorescence-activated cell sorting (FACS), and further cultured in the astroglial medium. Astroglia differentiated from Olig2−/GFP− and Olig2+/GFP+ NPCs were named as NPC-Astros and Olig2PC-Astros, respectively. The immature hiPSC-Astros, NPC-Astros, and Olig2PC-Astros cultured for 20 to 40 days in the astroglial medium were used in this study. To generate mature hPSC-Astros, the immature hPSC-Astros were further cultured in the medium containing DMEM/F12, 1×N2, 1×B27-RA, FGF1 (50 ng/mL; Peprotech), LIF (10 ng/mL; Sigma), and CNTF (10 ng/mL; Peprotech) for another 30 to 50 days. The hBrain-Astros were isolated from the cerebral cortex of fetal human brain (ScienCell; Catalog number: 1800) and cultured in the medium containing DMEM/F12, 1×N2 and 1% fetal bovine serum (FBS). A low concentration of FBS was added into the medium, as serum proteins may profoundly alter astrocyte properties (Foo et al., 2011; Smith et al., 1990; Zamanian et al., 2012). After being cultured in the presence of low centration of serum, the fetal tissue-derived hBrain-Astros become mature as described in previous studies (John, 2012; Lee et al., 1992). The hBrain-Astros from passage 8 to 10 were used in the work described in this example.

### Generation of Astrocyte-Conditioned Medium

To prepare astrocyte-conditioned medium (ACM), hPSC-Astros were cultured in 10-cm plates in astroglial differentiation medium containing BMP4 and FGF2 until confluent. The cells were then washed three times with warm DPBS and placed in minimal conditioning medium containing phenol-red-free DMEM/F12 and glutamine. After 3 days, ACM was collected, cell debris pelleted (4° C., 3,000 rpm for 5 min.) and then placed in centrifugal concentrators (Millipore) with a size cut off filter of 3 kDa. ACM was concentrated 50-fold. Protein concentration was determined by BCA assay (Thermo Scientific) and ACM was fed to primary mixed neuron/glia culture at 100 μg/mL. TIMP-1^{siRNA} ACM supplemented with TIMP-1 (10 ng/mL; Peprotech) was also fed to the primary culture.

### Primary Culture

Primary mixed cortical neuron/glia cultures were prepared from C57BL/6 mouse fetuses (Charles River) at embryonic day 15. The mixed neuron/glia cultures were maintained with culture medium consisted of Neurobasal medium and 1×B27 (Invitrogen). In order to maintain healthy primary cultures for over three weeks, cytosine arabinoside (1 μM, Sigma) was added to the medium from DIV 3 to 7 to eliminate or inhibit cell division (Gardner et al., 2012; Jiang et al., 2013a; Jiang et al., 2013c). At DIV 7, there were no nestin⁺ neural precursor cells detected in the cultures. Starting from DIV 7, the mixed neuron/glia cultures were fed with culture medium alone or culture medium plus concentrated ACM from hPSC-Astros and hBrain-Astros. The medium was changed every two days until experimentation (DIV 14 and DIV 21).

Primary mixed glial cultures were prepared from P1 to 2 C57BL/6 mice as previously described (Horiuchi et al., 2011). Briefly, the brains from PO mice were removed and submerged in ice-cold Leibovitz L-15 medium. The olfactory bulbs and hindbrains were removed. Meninges with blood vessels and choroid plexus were carefully peeled off. Remaining tissues were cut into small pieces and digested using trypsin (0.05% w/v) in HBSS. Cells were dissociated by trituration. Dissociated cells were collected and suspended in MEM alpha containing FBS (10% v/v) and plated in a tissue culture dish. Cells were maintained in growth medium (GM), a mixture of N1 medium (high glucose DMEM supplemented with 6 mM 1-glutamine, 10 ng/mL biotin, 5 μg/mL insulin, 50 μg/mL apotransferrin, 30 nM sodium selenite, 20 nM progesterone and 100 μM putrescine) and B104 neuroblastoma-conditioned medium (7:3 mixture v/v). Media was changed daily, and the mixed glial cultures were grown to confluency before purification.

### Immunopanning for Purification of Primary Mouse OPCs

Purification of mouse OPCs from mixed glial cultures was performed using a two-step approach of negative and positive affinity selection using surface markers, as described in a previous study (Chung et al., 2015). Separate low-adhesion dishes (10 cm) were first coated overnight with secondary antibodies that bind rat IgG or mouse IgG. These plates with secondary antibodies were then incubated with the respective primary antibodies: either anti-Thy1 rat IgG (clone 30H12) or anti-NG2 mouse IgG at least 2 hours before washing to remove unattached immunoglobulins. Mixed glial cultures were then trypsinized and resuspended in N1 medium containing 0.1% IgG-free BSA and plated on the anti-Thy1 IgG dish and incubated in a 37° C., 5% CO₂ humidified environment for a 30-minute negative selection. The non-adherent cell population from this dish was then removed and plated on the anti-NG2 IgG dish and incubated in a 37° C., 5% CO₂ humidified environment for a 30-minute positive selection. Adherent cells that represented primary OPCs were then collected by a rapid trypsinization using TrypLE (Invitrogen). OPCs were then plated in poly-L lysine-coated plates with OPC medium containing DMEM/F12, 1×N2, 1×B27, FGF2 (20 ng/mL) and platelet-derived growth factor-AA (PDGF-AA, 10 ng/mL; Peprotech). After 1 or 2 passages, the OPCs were fed with OPC medium alone or the OPC medium plus concentrated hiPSC-Astro ACM in the absence of FGF2 and PDGF-AA. Since hiPSCAstro ACM had more robust effects on OPC proliferation and differentiation in primary mixed neuron/glia culture (FIGS. 3C, 3E, 4B, and 4D) than in the purified OPC culture (FIGS. 6B-6E), the majority of the experiments were performed using the mixed neuron/glia culture system.

Cell proliferation assay

Cell proliferation was determined in 96-well plates using an MTT (3-(4,5 dimethylthiazolyl-2)-2,5-diphenyltetrazolium bromide)-based Cell Proliferation Kit I (Roche Diagnostics) (Jiang et al., 2010). HEK293 cells were used as a normalized control.

### Glutamate Clearance Assay

The method for measuring the decrease of glutamate over time was modified using the Glutamine/Glutamate Determination Kit (Sigma) (Jiang et al., 2013c). After subtraction of the blanks (0 glutamate added), the decrease in the media, or uptake of glutamate by cells, was reported as μM of glutamate per μg of protein after being normalized to the total protein in each well. The protein content was determined by a BCA protein assay (Thermo Scientific).

### Immunostaining

Cells were fixed with 4% paraformaldehyde and brain sections (18 μm thick) from P11-P60 mice and rats were processed for immunofluorescence staining (Liu et al., 2011b). The information for primary antibodies and dilutions are listed in Table 2. Secondary antibodies (all from Invitrogen), including Alexa 488 and 594 anti-rabbit, Alexa 488 and 594 anti-mouse, and Alexa 594 anti-rat, were all used at 1:1000. Slides or coverslips were mounted with the anti-fade Fluoromount-G medium containing 1,4′,6-diamindino-2-phenylindole dihydrochloride (DAPI) (Southern Biotechnology). Images were captured using a Nikon Eclipse C1 or Nikon A1 confocal laser-scanning microscope. The cells were counted with ImageJ software (NIH Image). For cultured cells, at least five fields of each coverslip were chosen randomly and three coverslips in each group were counted. For mouse brain sections, at least four consecutive sections from the same slice were evaluated for immunohistochemical quantifications, and the number of positive cells from each section was counted after a Z projection. At least 4 mice were used for each group. The analysis of fluorescence intensity of MBP staining was performed using ImageJ software. Dr. Lin Tian at University of California-Davis kindly provided us with the frozen sections of human brain tissues from normal individuals. The human brain tissues were de-identified by encoding with digital numbers and were originally obtained from the Human Brain and Spinal Fluid Resource Center at University of California, Los Angeles with patients' consent. The human brain tissues were derived from the frontal cerebral cortex of patients at the age of less than 6-month old.

### RNA Isolation and Quantitative PCR

Total RNA was prepared from cell pellets with RNAeasy kit (QIAGEN) (Jiang et al., 2010). Complementary DNA was prepared with the TaqMan Reverse Transcription Reagent (Life Technologies). The quantitative PCR (qPCR) was performed with TaqMan primers (Life Technologies) on a Roche Lightcycler 480. All primers used are listed in Table 3. All experimental samples were analyzed and normalized with the expression level of housekeeping gene glyceraldehyde 3-phosphate dehydrogenase (GAPDH). Relative quantification was performed by applying the 2^{-ΔΔCt} method (Livak and Schmittgen, 2001).

siRNA Transfection

hiPSC-Astros were plated in 6-well plates or 10-cm plates 24 hours prior to transfection with 40 nM Stealth RNAi, 40 nM Negative Universal Control Stealth, or 40 nM BLOCK-iT Fluorescent Oligo with Lipofectamine RNAiMAX Reagent (all from Invitrogen) according to the manufacturer's instructions. Transfections were performed in triplicate for each treatment. After 24 hours, transfection media was replaced with fresh growth media and incubation continued for an additional 24 hours. Transfection efficiency was assessed by visualizing uptake of the BLOCK-iT Fluorescent Oligo. In addition, at 48 hours after transfection, total RNA was harvested from individual wells of cells for qPCR analysis. Also, at 48 hours after transfection, additional duplicate wells or plates of cells were used for further experiments.

### Microarray Analysis and Heat Maps

Illumina bead array was performed for gene expression analysis (Campanelli et al., 2008; Liu et al., 2006). RNA was isolated from cultured cells using TRIzol (Invitrogen) and 100 ng total RNA was used for amplification and hybridization to an Illumina Human HT12_V4 chip according to the manufacturer's instructions (Illumina). The array was performed by the microarray core facility at UTHSC. Array data were processed using Illumina GenomeStudio software (Illumina). Background was subtracted and arrays were normalized using quantile. Gene expression levels were considered significant only when their detection p-value≤0.01. Heat maps of selected signaling pathway related genes were generated using R (A Language and Environment for Statistical Computing) or TMEV program in the TM4 software package. The matrix file containing the global gene expression of postnatal mouse astrocytes was obtained from NCBI Gene Expression Omnibus (GEO; GSE9566). Differential analysis between immature astrocytes (P1-8) and mature astrocytes (P17-30) were performed using package limma of R. |log (fold change) FC|>1 and P<0.05 were set as the cut-offs to screen out differentially expressed genes (DEGs). The expression values of DEGs were hierarchically clustered by package pheatmap of R.

### PVL Animal Model

By unilateral carotid ligation (UCL) followed with hypoxia, hypoxic/ischemic insults were induced in P6 mouse pups of Rag1−/− immunodeficient mice (B6.129S7-Rag1t^{m1Mom} on a C57BL/6 background, Jackson Lab) or P7 Long-Evans rat pups (Charles Rivers laboratories) (Follett et al., 2000). For the mouse PVL model, P6 mice were anesthetized under ice (indirect cooling) and then underwent UCL followed by a 1-hour recovery interval during which the pups were housed with the dam and kept on a thermal blanket to maintain body temperature at 33-34° C. Next, the pups were placed in an airproof isolation chamber with 6.0% O₂ for 35 minutes (Liu et al., 2011a; Shen et al., 2010). For the rat PVL model, P7 rats were anesthetized with 1.5% isoflurane. Then, the injury was induced by UCL followed by hypoxia (6.0% O₂ for 1 hour) (Follett et al., 2000).

### Cell Transplantation

Mice were randomized to the vehicle (PBS) or cell transplantation groups. Human iPSC-Astros were suspended at a final concentration of 100,000 cells per μL in PBS. The mouse pups were first cryoanesthetized and 100,000 human astrocytes in 1 μL PBS or PBS alone were injected into a location adjacent to the injury site (anteroposterior: 2 mm, lateral: 2 mm, dorsoventral: 2 mm with reference to Bregma). A Hamilton syringe and needle were used to deliver cells by insertion through the skull into the target site (Chen et al., 2014). The pups were weaned at 3 weeks.

### Intranasal Administration

Rats were randomized to the vehicle (control minimal conditioning medium) or ACM groups. The control medium or concentrated ACM (2.5 to 3 mg/mL) was administered intranasally at no more than 5 μL with increments 5-10 minutes apart for a total of 3 μg/g. The rat was held ventral side up, and a fine pipette tip was inserted into either nare. Control medium or ACM was slowly administered and the rat was held for 1-2 minutes to ensure absorption. Control medium or ACM was administered every 12 hours from P8 to P11.

### ELISA Analysis of Human TIMP-1

Rat pups at P11 were decapitated at 1 hour after the last dose of control medium or ACM. The brains were separated into three parts: olfactory bulb (OB), frontal brain (FB), and posterior brain (PB). The FB and PB were separated coronally at about the bregma level. Brain tissues were weighed, and homogenized in PBS containing protease inhibitors using sonication. Homogenates were centrifuged (14,000 rpm for 20 minutes at 4° C.), and supernatant was extracted. Protein concentrations were determined by BCA assay (Thermo Scientific), and samples were diluted with sterile PBS. Samples were then analyzed using a human TIMP-1 ELISA kit (BosterBio). Plates were read on a plate reader at 450 nm (Molecular Devices).

### Electron Microscopy

Mice were perfused with saline followed by 2% paraformaldehyde plus 2.5% glutaraldehyde in 0.1M phosphate buffer (PB, pH 7.4). Brains were immediately removed, and postfixed in the fixative solution for a week at 4° C. Brain blocks were then washed in PB and cut sagittally on a vibratome (Leica) at a thickness of 60 μm. The sections were collected in cold 0.1M PB. To maintain consistency of the samples, all vibratome sections were processed simultaneously for EM (Liu and Schumann, 2014). Briefly, sections were osmicated in 2% OₛO₄ in 0.1 M PB for 20 minutes, washed and dehydrated in grade ethanol and 100% acetone. Sections were flat embedded in Araldite and polymerized at 70° C. in an oven for 2 days. Embedded sections were examined under light microscope to identify the corpus callosum regions. The mid-anterior region above the hippocampus was selected for ultrathin sectioning (70 nm; Leica Ultracut). Thin sections were collected on Formva coated single slot copper grids, which were counterstained with uranyl acetate and lead citrate. Thin sections were examined under a Philips CM120 Electron Microscope at 80 kV. For each group, three cases of each brain sample were examined. For each sample case, at least 20 fields were taken from the selected corpus callosum region. For counting myelinated axons, images were taken at approximately 4800×, so that each myelinated axon can be identified. Axons were also imaged at 20,000× to 25, 800× to get a better view of myelin sheaths. EM images were captured using a 2K×2K high resolution CCD camera (Gatan, Pleasanton, Calif.). Images were processed using DigitalMicrograph (Gatan). EM images were analyzed using ImageJ software. The density of myelinated axons has been used to indicate myelin injury and recovery (Keirstead et al., 2005). In this study, the density of myelinated axons was calculated as the number of myelinated axons per μm². The number of myelinated axons in a 10 to 20 defined unit area (imaging field of 104.4 μm² at ×4,800) was counted. In each unit area from P60 mouse brains, about 780 to 1,068 myelinated axons were counted. To quantify the g ratio, 50-100 myelinated axons were randomly chosen from each case and measured (Jiang et al., 2013b). The g ratio was calculated as following: the diameter of the axon caliber (a) divided by diameter of the myelinated axon fiber (A): g ratio=a/A.

### Morris Water-Maze Test

Mice were placed in the water by hand facing the wall at one random start location out of four, and were allowed to find the submerged platform within 60 seconds. A trial was terminated if the mouse was able to find the platform. If the mouse did not find the hidden platform within 60 seconds, it was guided onto the platform with a stick. The mouse was allowed to stay on the platform for 20 seconds before being removed. The training was repeated from each of the four randomized starting locations, and 1 hour was allowed between sessions. The latency time and swimming distance were monitored by an overhead video camera and analyzed by an automated tracking system (Harvard Apparatus). Four hours after the final training trial, each mouse was subjected to a probe trial (60 seconds) in which no platform was present. The mouse was placed in the water at the same random start location, and the time spent in the quadrant that formerly contained the platform was recorded to assess the level of spatial bias.

### References for Supplemental Experimental Procedures

Campanelli, J. T., Sandrock, R. W., Wheatley, W., Xue, H., Zheng, J., Liang, F., Chesnut, J. D., Zhan, M., Rao, M. S., and Liu, Y. (2008). Expression profiling of human glial precursors. BMC developmental biology 8, 102.

Chen, C., Jiang, P., Xue, H., Peterson, S. E., Tran, H. T., McCann, A. E., Parast, M. M., Li, S., Pleasure, D. E., Laurent, L. C., et al. (2014). Role of astroglia in Down's syndrome revealed by patient-derived human-induced pluripotent stem cells. Nat Commun 5, 4430.

Chung, S. H., Biswas, S., Selvaraj, V., Liu, X. B., Sohn, J., Jiang, P., Chen, C., Chmilewsky, F., Marzban, H., Horiuchi, M., et al. (2015). The p38 alpha mitogen-activated protein kinase is a key regulator of myelination and remyelination in the CNS. Cell Death Dis 6, e1748.

Follett, P. L., Rosenberg, P. A., Volpe, J. J., and Jensen, F. E. (2000). NBQX attenuates excitotoxic injury in developing white matter. J Neurosci 20, 9235-9241.

Foo, L. C., Allen, N. J., Bushong, E. A., Ventura, P. B., Chung, W. S., Zhou, L., Cahoy, J. D., Daneman, R., Zong, H., Ellisman, M. H., et al. (2011). Development of a method for the purification and culture of rodent astrocytes. Neuron 71, 799-811.

Gardner, A., Jukkola, P., and Gu, C. (2012). Myelination of rodent hippocampal neurons in culture. Nature protocols 7, 1774-1782.

Horiuchi, M., Itoh, A., Pleasure, D., Ozato, K., and Itoh, T. (2011). Cooperative contributions of interferon regulatory factor 1 (IRF1) and IRF8 to interferon-gamma-mediated cytotoxic effects on oligodendroglial progenitor cells. J Neuroinflammation 8, 8.

Jiang, P., Chen, C., Liu, X. B., Selvaraj, V., Liu, W., Feldman, D. H., Liu, Y., Pleasure, D. E., Li, R. A., and Deng, W. (2013a). Generation and characterization of spiking and non-spiking oligodendroglial progenitor cells from embryonic stem cells. Stem cells.

Jiang, P., Chen, C., Liu, X. B., Selvaraj, V., Liu, W., Feldman, D. H., Liu, Y., Pleasure, D. E., Li, R. A., and Deng, W. (2013b). Generation and characterization of spiking and nonspiking oligodendroglial progenitor cells from embryonic stem cells. Stem cells 31, 2620-2631.

Jiang, P., Chen, C., Wang, R., Chechneva, O. V., Chung, S. H., Rao, M. S., Pleasure, D. E., Liu, Y., Zhang, Q., and Deng, W. (2013c). hESC-derived Olig2+ progenitors generate a subtype of astroglia with protective effects against ischaemic brain injury. Nat Commun 4, 2196.

Jiang, P., Rushing, S. N., Kong, C. W., Fu, J., Lieu, D. K., Chan, C. W., Deng, W., and Li, R. A. (2010). Electrophysiological properties of human induced pluripotent stem cells. American journal of physiology Cell physiology 298, C486-495.

John, G. R. (2012). Investigation of astrocyte—oligodendrocyte interactions in human cultures. Methods Mol Biol 814, 401-414.

Keirstead, H. S., Nistor, G., Bernal, G., Totoiu, M., Cloutier, F., Sharp, K., and Steward, O. (2005). Human embryonic stem cell-derived oligodendrocyte progenitor cell transplants remyelinate and restore locomotion after spinal cord injury. J Neurosci 25, 4694-4705.

Lee, S. C., Liu, W., Brosnan, C. F., and Dickson, D. W. (1992). Characterization of primary human fetal dissociated central nervous system cultures with an emphasis on microglia. Lab Invest 67, 465-476.

Liu, W., Shen, Y., Plane, J. M., Pleasure, D. E., and Deng, W. (2011a). Neuroprotective potential of erythropoietin and its derivative carbamylated erythropoietin in periventricular leukomalacia. Experimental neurology 230, 227-239.

Liu, X. B., and Schumann, C. M. (2014). Optimization of electron microscopy for human brains with long-term fixation and fixed-frozen sections. Acta neuropathologica communications 2, 42.

Liu, Y., Jiang, P., and Deng, W. (2011b). OLIG gene targeting in human pluripotent stem cells for motor neuron and oligodendrocyte differentiation. Nature protocols 6, 640-655.

Liu, Y., Shin, S., Zeng, X., Zhan, M., Gonzalez, R., Mueller, F. J., Schwartz, C. M., Xue, H., Li, H., Baker, S. C., et al. (2006). Genome wide profiling of human embryonic stem cells (hESCs), their derivatives and embryonal carcinoma cells to develop base profiles of U.S. Federal government approved hESC lines. BMC developmental biology 6, 20.

Livak, K. J., and Schmittgen, T. D. (2001). Analysis of relative gene expression data using real-time quantitative PCR and the 2(-Delta Delta C(T)) Method. Methods 25, 402-408.

Shen, Y., Plane, J. M., and Deng, W. (2010). Mouse models of periventricular leukomalacia. Journal of visualized experiments: JoVE.

Smith, G. M., Rutishauser, U., Silver, J., and Miller, R. H. (1990). Maturation of astrocytes in vitro alters the extent and molecular basis of neurite outgrowth. Dev Biol 138, 377-390.

Zamanian, J. L., Xu, L., Foo, L. C., Noun, N., Zhou, L., Giffard, R. G., and Barres, B. A. (2012). Genomic analysis of reactive astrogliosis. J Neurosci 32, 6391-6410.

TABLE 1
Astrocyte-secreted factors reported to regulate
OPC differentiation and proliferation

| | Human | | |
|---|---|---|---|
| | Gene | Effects | |
| Factors | Symbol | on OPCs | References |
| TGFβ | TGFB1, | Promote | (Palazuelos et al., 2014) |
| | TGFB2, | differentiation | |
| | TGFB3 | | |
| Neuregulin | NRG1, | Promote | (Lundgaard et al., 2013; |
| | NRG2, | differentiation | Taveggia et al., 2008) |
| | NRG3, | | |
| | NRG4 | | |
| Thrombospondin | THBS1, | Promote | (Scott-Drew and |
| | THBS2, | differentiation | ffrench-Constant, 1997) |
| | THBS3, | | |
| | THBS4 | | |
| γ-secretase | APH1A, | Promote | (Watkins et al., 2008) |
| | APH1B | differentiation | |
| Metallopeptidase | TMP1 | Promote | (Crocker et al., 2006; |
| inhibitor 1 | | differentiation | Moore et al., 2011) |
| (TIMP1) | | | |
| Ciliary | CNTF | Promote | (Sendtner et al., 1994; |
| neurotrophic | | differentiation | Stankoff et al., 2002) |
| factor (CNTF) | | | |
| Leukemia | LIF | Promote | (Stankoff et al., 2002) |
| inhibitory | | differentiation | |
| factor (LIF) | | | |
| IL-6 | IL-6 | Promote | (Stankoff et al., 2002) |
| | | differentiation | |
| Osteopointin | SPP1 | Promote | (Selvaraju et al., 2004) |
| | | differentiation | |
| Insulin-like | IGF-1 | Promote | (Ballotti et al., 1987) |
| growth | | differentiation | |
| factor 1 | | | |
| (IGF-1) | | | |
| Brain-derived | BDNF | Promote | (Lundgaard et al., 2013; |
| neurotrophic | | differentiation | Xiao et al., 2010) |
| factor (BDNF) | | | |
| Neurotrophin 3 | NTF3 | Promote | (Kumar et al., 2007) |
| (NT3) | | differentiation | |
| Glial | GDNF | Promote | (Iannotti et al., 2003) |
| cell-derived | | differentiation | |
| neurotrophic | | | |
| factor (GDNF) | | | |
| Nerve growth | NGF | Promote | (Yin et al., 2012) |
| factor (NGF) | | differentiation | |
| Laminin | LAMA, | Promote | (Buttery and ffrench- |
| | LAMB, | differentiation | Constant, 1999) |
| | LAMC | | |
| Transforming | TGFA | Inhibit | (Deng et al., 2010) |
| growth factor, | | differentiation | |
| alpha (TGFα) | | | |
| Interleukin-1 | IL1B | Inhibit | (Deng et al., 2010) |
| beta (IL1β) | | differentiation | |
| Bone | BMP2, | Inhibit | (See and Grinspan, 2009; |
| morphogenetic | BMP4 | differentiation | Wang et al., 2011) |
| proteins 2/4 | | | |
| (BMP2/4) | | | |
| Tenascin C | TnC | Inhibit | (Czopka et al., 2010) |
| | | differentiation | |
| Hyaluronan | HAS1, | Inhibit | (Sloane et al., 2010) |
| synthase | HAS2, | differentiation | |
| | HAS3 | | |
| Chemokine | CXCL10, | Inhibit | (Kerstetter et al., 2009; |
| (C-X-C motif) | CXCL12, | differentiation | Maysami et al., 2006; |
| ligands | CXCL1 | | Nash et al., 2011) |
| (CXCL10, | | | |
| CXCL12, | | | |
| CXCL1) | | | |
| Connective | CTGF | Inhibit | (Stritt et al., 2009) |
| tissue growth | | differentiation | |
| factor (CTGF) | | | |
| Leucine rich | LINGO1 | Inhibit | (Satoh et al., 2007) |
| repeat and Ig | | differentiation | |
| domain | | | |
| containing 1 | | | |
| (Lingo-1) | | | |
| Endothelin-1 | EDN1 | Inhibit | (Hammond et al., 2014) |
| (ET1) | | differentiation | |
| Platelet-derived | PDGFA, | Promote | (Bogler et al., 1990) |
| growth factor | PDGFB, | proliferation | |
| (PDGF) | PDGFC, | of OPCs | |
| | PDGFD | | |
| Fibroblast | FGF-2 | Promote | (Bogler et al., 1990) |
| growth | | proliferation | |
| factor-2 | | of OPCs | |

### References for Table 1

Ballotti, R., Nielsen, F. C., Pringle, N., Kowalski, A., Richardson, W. D., Van Obberghen, E., and Gammeltoft, S. (1987). Insulin-like growth factor I in cultured rat astrocytes: expression of the gene, and receptor tyrosine kinase. The EMBO journal 6, 3633-3639.

Bogler, O., Wren, D., Barnett, S. C., Land, H., and Noble, M. (1990). Cooperation between two growth factors promotes extended self-renewal and inhibits differentiation of oligodendrocyte-type-2 astrocyte (O-2A) progenitor cells. Proc Natl Acad Sci U S A 87, 6368-6372.

Buttery, P. C., and ffrench-Constant, C. (1999). Laminin-2/integrin interactions enhance myelin membrane formation by oligodendrocytes. Molecular and cellular neurosciences 14, 199-212.

Crocker, S. J., Whitmire, J. K., Frausto, R. F., Chertboonmuang, P., Soloway, P. D., Whitton, J. L., and Campbell, I. L. (2006). Persistent macrophage/microglial activation and myelin disruption after experimental autoimmune encephalomyelitis in tissue inhibitor of metalloproteinase-1-deficient mice. The American journal of pathology 169, 2104-2116.

Czopka, T., von Holst, A., ffrench-Constant, C., and Faissner, A. (2010). Regulatory mechanisms that mediate tenascin C-dependent inhibition of oligodendrocyte precursor differentiation. J Neurosci 30, 12310-12322.

Deng, Y. Y., Lu, J., Ling, E. A., and Kaur, C. (2010). Microglia-derived macrophage colony stimulating factor promotes generation of proinflammatory cytokines by astrocytes in the periventricular white matter in the hypoxic neonatal brain. Brain pathology 20, 909-925.

Hammond, T. R., Gadea, A., Dupree, J., Kerninon, C., Nait-Oumesmar, B., Aguirre, A., and Gallo, V. (2014). Astrocyte-derived endothelin-1 inhibits remyelination through notch activation. Neuron 81, 588-602.

Iannotti, C., Li, H., Yan, P., Lu, X., Wirthlin, L., and Xu, X. M. (2003). Glial cell line-derived neurotrophic factor-enriched bridging transplants promote propriospinal axonal regeneration and enhance myelination after spinal cord injury. Experimental neurology 183, 379-393.

Kerstetter, A. E., Padovani-Claudio, D. A., Bai, L., and Miller, R. H. (2009). Inhibition of CXCR2 signaling promotes recovery in models of multiple sclerosis. Experimental neurology 220, 44-56.

Kumar, S., Biancotti, J. C., Yamaguchi, M., and de Vellis, J. (2007). Combination of growth factors enhances remyelination in a cuprizone-induced demyelination mouse model. Neurochemical research 32, 783-797.

Lundgaard, I., Luzhynskaya, A., Stockley, J. H., Wang, Z., Evans, K. A., Swire, M., Volbracht, K., Gautier, H. O., Franklin, R. J., Charles, F.-C., et al. (2013). Neuregulin and BDNF induce a switch to NMDA receptor-dependent myelination by oligodendrocytes. PLoS biology 11, e1001743.

Maysami, S., Nguyen, D., Zobel, F., Pitz, C., Heine, S., Hopfner, M., and Stangel, M. (2006). Modulation of rat oligodendrocyte precursor cells by the chemokine CXCL12. Neuroreport 17, 1187-1190.

Moore, C. S., Milner, R., Nishiyama, A., Frausto, R. F., Serwanski, D. R., Pagarigan, R. R., Whitton, J. L., Miller, R. H., and Crocker, S. J. (2011). Astrocytic tissue inhibitor of metalloproteinase-1 (TIMP-1) promotes oligodendrocyte differentiation and enhances CNS myelination. J Neurosci 31, 6247-6254.

Nash, B., Thomson, C. E., Linington, C., Arthur, A. T., McClure, J. D., McBride, M. W., and Barnett, S. C. (2011). Functional duality of astrocytes in myelination. J Neurosci 31, 13028-13038.

Palazuelos, J., Klingener, M., and Aguirre, A. (2014). TGFbeta signaling regulates the timing of CNS myelination by modulating oligodendrocyte progenitor cell cycle exit through SMAD3/4/FoxO1/Sp1. J Neurosci 34, 7917-7930.

Satoh, J., Tabunoki, H., Yamamura, T., Arima, K., and Konno, H. (2007). TROY and LINGO-1 expression in astrocytes and macrophages/microglia in multiple sclerosis lesions. Neuropathology and applied neurobiology 33, 99-107.

Scott-Drew, S., and ffrench-Constant, C. (1997). Expression and function of thrombospondin-1 in myelinating glial cells of the central nervous system. Journal of neuroscience research 50, 202-214.

See, J. M., and Grinspan, J. B. (2009). Sending mixed signals: bone morphogenetic protein in myelination and demyelination. Journal of neuropathology and experimental neurology 68, 595-604.

Selvaraju, R., Bernasconi, L., Losberger, C., Graber, P., Kadi, L., Avellana-Adalid, V., Picard-Riera, N., Baron Van Evercooren, A., Cirillo, R., Kosco-Vilbois, M., et al. (2004). Osteopontin is upregulated during in vivo demyelination and remyelination and enhances myelin formation in vitro. Molecular and cellular neurosciences 25, 707-721.

Sendtner, M., Carroll, P., Holtmann, B., Hughes, R. A., and Thoenen, H. (1994). Ciliary neurotrophic factor. Journal of neurobiology 25, 1436-1453.

Sloane, J. A., Batt, C., Ma, Y., Harris, Z. M., Trapp, B., and Vartanian, T. (2010). Hyaluronan blocks oligodendrocyte progenitor maturation and remyelination through TLR2. Proc Natl Acad Sci USA 107, 11555-11560.

Stankoff, B., Aigrot, M. S., Noel, F., Wattilliaux, A., Zalc, B., and Lubetzki, C. (2002). Ciliary neurotrophic factor (CNTF) enhances myelin formation: a novel role for CNTF and CNTF-related molecules. J Neurosci 22, 9221-9227.

Stritt, C., Stern, S., Harting, K., Manke, T., Sinske, D., Schwarz, H., Vingron, M., Nordheim, A., and Knoll, B. (2009). Paracrine control of oligodendrocyte differentiation by SRF-directed neuronal gene expression. Nature neuroscience 12, 418-427.

Taveggia, C., Thaker, P., Petrylak, A., Caporaso, G. L., Toews, A., Falls, D. L., Einheber, S., and Salzer, J. L. (2008). Type III neuregulin-1 promotes oligodendrocyte myelination. Glia 56, 284-293.

Wang, Y., Cheng, X., He, Q., Zheng, Y., Kim, D. H., Whittemore, S. R., and Cao, Q. L. (2011). Astrocytes from the contused spinal cord inhibit oligodendrocyte differentiation of adult oligodendrocyte precursor cells by increasing the expression of bone morphogenetic proteins. J Neurosci 31, 6053-6058.

Watkins, T. A., Emery, B., Mulinyawe, S., and Barres, B. A. (2008). Distinct stages of myelination regulated by gamma-secretase and astrocytes in a rapidly myelinating CNS coculture system. Neuron 60, 555-569.

Xiao, J., Wong, A. W., Willingham, M. M., van den Buuse, M., Kilpatrick, T. J., and Murray, S. S. (2010). Brain-derived neurotrophic factor promotes central nervous system myelination via a direct effect upon oligodendrocytes. Neuro-Signals 18, 186-202.

Yin, L. L., Lin, L. L., Zhang, L., and Li, L. (2012). Epimedium flavonoids ameliorate experimental autoimmune encephalomyelitis in rats by modulating neuroinflammatory and neurotrophic responses. Neuropharmacology 63, 851-862.

TABLE 2
Antibodies used

| Antibodies | Vendor/Catalog #. | Type | Dilution |
|---|---|---|---|
| Caspr | NeuroMab/Clone K65/35 | Mouse IgG1 | 1:100 |
| CC1 | Millipore/OP-80 | Mouse IgG | 1:200 |
| GFAP | Millipore/AB5804 | Rabbit IgG | 1:1000 |
| HOXB4 | DSHB/I12 | Rat IgG | 1:200 |
| Human CD44 | Abcam/ab6124 | Mouse IgG | 1:1000 |
| Human nuclei | Millipore/MAB4383 | Mouse IgG | 1:100 |
| Human TIMP-1 | BosterBio/PA1385 | Rabbit IgG | 1:200 |
| Ki67 | Cell signaling/9449 | Mouse IgG | 1:400 |
| MAP2 | Millipore/AB3418 | Mouse IgG | 1:500 |
| MBP | Millipore/MAB386 | Rat IgG | 1:100 |
| NF1A | Active Motif/39329 | Rabbit IgG | 1:500 |
| NG2 | Millipore/AB5320 | Rabbit IgG | 1:200 |
| Nkx2.1 | Millipore/MAB5460 | Mouse IgG | 1:500 |
| Olig1 | Phosphosolutions/1537 | Rabbit IgG | 1:200 |
| Olig1 | R&D/MAB2417 | Mouse IgG | 1:10 |
| Olig2 | Phosphosolutions/1538 | Rabbit IgG | 1:1000 |
| Otx2 | R&D/AF2418 | Mouse IgG | 1:1000 |
| PDGFRα | Santa Cruz | Rabbit IgG | 1:50 |
| | Biotechnology/SC338 | | |
| s100β | Sigma/S2532 | Mouse IgG | 1:1000 |
| Vimentin | Cell signaling/#3932 | Rabbit IgG | 1:1000 |
| βIIItubulin | Millipore/MAB1637 | Mouse IgG | 1:200 |
| βIIItubulin | Covance/PRB-435P | Rabbit IgG | 1:3000 |
| βIV spectrin | NeuroMab/Clone N393/76 | Mouse IgG2b | 1:100 |

TABLE 3
TaqMan primers

| Gene | Gene expression assay catalog number |
|---|---|
| CD44 | Hs01075861_m1 |
| Cnp | Mm01306640_m1 |
| GAPDH | Hs02758991_g1 |
| Gapdh | Mm99999915_g1 |
| EAAT1 | Hs00188193_m1 |
| EAAT2 | Hs01102423_m1 |
| Mbp | Mm01266402_m1 |
| NF1A | Hs00231172_m1 |
| Olig1 | Mm00497537_s1 |
| Pdgfra | Mm00440701_m1 |
| Plp1 | Mm01297210_m1 |
| TIMP-1 | Hs00171558_m1 |
| VIMENTIN | Hs00185584_m1 |

### V. Exemplary Embodiments

Exemplary embodiments provided in accordance with the presently disclosed subject matter include, but are not limited to, the claims and the following embodiments:

1. A method for preventing or treating a demyelinating disease in a subject, the method comprising administering to the subject a therapeutically effective amount of immature astrocytes.

2. The method of embodiment 1, wherein administration comprises transplanting the immature astrocytes into injured tissue in the subject.

3. The method of embodiment 1 or 2, wherein about 1,000,000 to about 10,000,000 immature astrocytes are administered to the subject.

4. The method of embodiment 3, wherein about 2,250,000 to about 4,500,000 immature astrocytes are administered to the subject.

5. The method of any one of embodiments 1 to 4, wherein the immature astrocytes are suspended in a pharmaceutically acceptable carrier prior to administration.

6. The method of embodiment 5, wherein the pharmaceutically acceptable carrier comprises phosphate-buffered saline.

7. The method of embodiment 5 or 6, wherein the immature astrocytes are present at a concentration of about 50,000 to about 100,000 cells per microliter in the suspension.

8. The method of any one of embodiments 1 to 7, wherein the demyelinating disease is selected from the group consisting of periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof.

9. The method of any one of embodiments 1 to 8, wherein the subject is a human.

10. The method of any one of embodiments 1 to 9, wherein treating the subject reduces or eliminates one or more signs or symptoms of a demyelinating disease.

11. The method of any one of embodiments 1 to 9, wherein the subject does not have signs or symptoms of a demyelinating disease.

12. The method of any one of embodiments 1 to 11, wherein the subject has one or more risk factors for a demyelinating disease.

13. The method of any one of embodiments 1 to 12, wherein the immature astrocytes are not co-administered with another cell type.

14. The method of any one of embodiments 1 to 13, wherein the immature astrocytes are derived from a pluripotent stem cell.

15. The method of embodiment 14, wherein the pluripotent stem cell is a human pluripotent stem cell.

16. The method of embodiment 14 or 15, wherein the pluripotent stem cell is an induced pluripotent stem cell.

17. The method of embodiment 16, wherein the induced pluripotent stem cell is derived from a cell obtained from the subject.

18. The method of embodiment 14 or 15, wherein the pluripotent stem cell is an embryonic stem cell.

19. The method of any one of embodiments 1 to 18, further comprising determining the presence or level of one or biomarkers expressed by the immature astrocytes, wherein the presence or level of the one or more biomarkers is determined before administration.

20. The method of embodiment 19, wherein the one or more biomarkers is selected from the group consisting of tissue inhibitor of metalloproteinase-1 (TIMP-1), glial fibrillary acidic protein (GFAP), S100 calcium-binding protein B (S100β), CD44, vimentin, nuclear factor 1 A-type (NF1A), excitatory amino acid transporter 1 (EAAT1), and a combination thereof.

21. The method of embodiment 19 or 20, wherein the presence or level of the one or more biomarkers is compared to a control.

22. The method of embodiment 21, wherein the control is a mature astrocyte.

23. The method of embodiment 22, wherein the level of TIMP-1, CD44, vimentin, NF1A, and/or GFAP is higher in the immature astrocytes than in the mature astrocyte control.

24. The method of embodiment 22, wherein the level of EAAT1 is lower in the immature astrocytes than in the mature astrocyte control.

25. The method of any one of embodiments 19 to 24, wherein the presence or level of the one or more biomarkers is determined by a method selected from the group consisting of immunohistochemistry, quantitative PCR, a glutamate transport assay, and a combination thereof.

26. A method for reducing demyelination, inducing remyelination, promoting oligodendroglial progenitor cell (OPC) proliferation, and/or promoting oligodendrocyte differentiation in a subject, the method comprising administering to the subject a therapeutically effective amount of immature astrocytes.

27. The method of embodiment 26, wherein administration comprises transplanting the immature astrocytes into injured tissue in the subject.

28. The method of embodiment 26 or 27, wherein about 1,000,000 to about 10,000,000 immature astrocytes are administered to the subject.

29. The method of embodiment 28, wherein about 2,250,000 to about 4,500,000 immature astrocytes are administered to the subject.

30. The method of any one of embodiments 26 to 29, wherein the immature astrocytes are suspended in a pharmaceutically acceptable carrier prior to administration.

31. The method of embodiment 30, wherein the pharmaceutically acceptable carrier comprises phosphate-buffered saline.

32. The method of embodiment 30 or 31, wherein the immature astrocytes are present at a concentration of about 50,000 to about 100,000 cells per microliter in the suspension.

33. The method of any one of embodiments 26 to 32, wherein the subject is a human.

34. The method of any one of embodiments 26 to 33, wherein the subject has one or more risk factors for a demyelinating disease.

35. The method of any one of embodiments 26 to 34, wherein reducing demyelination, inducing remyelination, promoting oligodendroglial progenitor cell (OPC) proliferation, and/or promoting oligodendrocyte differentiation in the subject reduces or eliminates one or more signs or symptoms of a demyelinating disease.

36. The method of any one of embodiments 26 to 34, wherein the subject does not have signs or symptoms of a demyelinating disease.

37. The method of any one of embodiments 34 to 36, wherein the demyelinating disease is selected from the group consisting of periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof.

38. The method of any one of embodiments 26 to 37, wherein the immature astrocytes are not co-administered with another cell type.

39. The method of any one of embodiments 26 to 38, wherein the immature astrocytes are derived from a pluripotent stem cell.

40. The method of embodiment 39, wherein the pluripotent stem cell is a human pluripotent stem cell.

41. The method of embodiment 39 or 40, wherein the pluripotent stem cell is an induced pluripotent stem cell.

42. The method of embodiment 41, wherein the induced pluripotent stem cell is derived from a cell obtained from the subject.

43. The method of embodiment 39 or 40, wherein the pluripotent stem cell is an embryonic stem cell.

44. The method of any one of embodiments 26 to 43, further comprising determining the presence or level of one or biomarkers expressed by the immature astrocytes, wherein the presence or level of the one or more biomarkers is determined before administration.

45. The method of embodiment 44, wherein the one or more biomarkers is selected from the group consisting of tissue inhibitor of metalloproteinase-1 TIMP-1, glial fibrillary acidic protein (GFAP), S100 calcium-binding protein B (S100β), CD44, vimentin, nuclear factor 1 A-type (NF1A), excitatory amino acid transporter 1 (EAAT1), and a combination thereof.

46. The method of embodiment 44 or 45, wherein the presence or level of the one or more biomarkers is compared to a control.

47. The method of embodiment 46, wherein the control is a mature astrocyte.

48. The method of embodiment 47, wherein the level of TIMP-1, CD44, vimentin, NF1A, and/or GFAP is higher in the immature astrocytes than in the mature astrocyte control.

49. The method of embodiment 47, wherein the level of EAAT1 is lower in the immature astrocytes than in the mature astrocyte control.

50. The method of any one of embodiments 44 to 49, wherein the presence or level of the one or more biomarkers is determined by a method selected from the group consisting of immunohistochemistry, quantitative PCR, a glutamate transport assay, and a combination thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

1. A method for preventing or treating a demyelinating disease in a subject, the method comprising administering to the subject a therapeutically effective amount of immature astrocytes.

2. The method of claim 1, wherein administration comprises transplanting the immature astrocytes into injured tissue in the subject.

3. The method of claim 1, wherein about 1,000,000 to about 10,000,000 immature astrocytes are administered to the subject.

4. (canceled)

5. The method of claim 1, wherein the immature astrocytes are suspended in a pharmaceutically acceptable carrier prior to administration.

6-7. (canceled)

8. The method of claim 1, wherein the demyelinating disease is selected from the group consisting of periventricular leukomalacia, multiple sclerosis, acute disseminated encephalomyelitis, chronic inflammatory demyelinating polyneuropathy, adrenoleukodystrophy, adenomyeloneuropathy, Leber's hereditary optic atrophy, HTLV-associated myelopathy, Guillain-Barre syndrome, phenylketonuria, Tay-Sachs disease, Niemann-Pick disease, Gaucher's disease, Hurler's syndrome, Krabbe's disease, Pelizaeus-Merzbacher disease, cerebral palsy, and a combination thereof.

9. (canceled)

10. The method of claim 1, wherein treating the subject reduces or eliminates one or more signs or symptoms of the demyelinating disease.

11. The method of claim 1, wherein the subject does not have signs or symptoms of the demyelinating disease.

12. The method of claim 1, wherein the subject has one or more risk factors for the demyelinating disease.

13. (canceled)

14. The method of claim 1, wherein the immature astrocytes are derived from a pluripotent stem cell.

15. The method of claim 14, wherein the pluripotent stem cell is a human pluripotent stem cell.

16. The method of claim 14, wherein the pluripotent stem cell is an induced pluripotent stem cell.

17. The method of claim 16, wherein the induced pluripotent stem cell is derived from a cell obtained from the subject.

18-25. (canceled)

26. A method for reducing demyelination, inducing remyelination, promoting oligodendroglial progenitor cell (OPC) proliferation, and/or promoting oligodendrocyte differentiation in a subject, the method comprising administering to the subject a therapeutically effective amount of immature astrocytes.

27. The method of claim 26, wherein administration comprises transplanting the immature astrocytes into injured tissue in the subject.

28. The method of claim 26, wherein about 1,000,000 to about 10,000,000 immature astrocytes are administered to the subject.

29. (canceled)

30. The method of claim 26, wherein the immature astrocytes are suspended in a pharmaceutically acceptable carrier prior to administration.

31-38. (canceled)

39. The method of claim 26, wherein the immature astrocytes are derived from a pluripotent stem cell.

40. The method of claim 39, wherein the pluripotent stem cell is a human pluripotent stem cell.

41. The method of claim 39, wherein the pluripotent stem cell is an induced pluripotent stem cell.

42. The method of claim 41, wherein the induced pluripotent stem cell is derived from a cell obtained from the subject.

43-50. (canceled)
